SEQUENCE LISTING (50 sequences)

SEQ ID NO: 1 - protein, 513 aa, Homo sapiens
  human ActRIIA precursor polypeptide
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 2 - protein, 115 aa, Homo sapiens
  human ActRIIA soluble (extracellular), processed polypeptide sequence
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 3 - protein, 100 aa, Artificial Sequence
  human ActRIIA soluble (extracellular), processed polypeptide sequence with the C-terminal 15 amino acids deleted
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 4 - DNA, 1542 bp, Homo sapiens
  nucleic acid sequence of human ActRIIA precursor protein
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccagagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactagggttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttcgaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtctatga

SEQ ID NO: 5 - DNA, 345 bp, Homo sapiens
  human ActRIIA soluble (extracellular) polypeptide
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccagagatggaagtcacacagcccacttcaaatccagttacacctaagccaccc

SEQ ID NO: 6 - protein, 228 aa, Artificial Sequence
  fusion protein comprising a soluble extracellular domain of ActRIIA fused to an Fc domain
  VARIANT 44: Xaa = Asp or Ala
  VARIANT 102: Xaa = Lys or Ala
  VARIANT 215: Xaa = Asn or Ala
THTCPPCPAPELLGGPSVFLFPPKPKDTLMISRTPEVTCVVVDXVSHEDPEVKFNWYVDGVEVHNAKTKPREEQYNSTYRVVSVLTVLHQDWLNGKEYKCKXVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNXHYTQKSLSLSPGK

SEQ ID NO: 7 - protein, 344 aa, Artificial Sequence
  Extracellular domain of human ActRIIA fused to a human Fc domain
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 21 aa, Artificial Sequence
  Leader sequence of Honey bee mellitin (HBML)
MKFLVNVALVFMVVYISYIYA

SEQ ID NO: 9 - protein, 22 aa, Artificial Sequence
  Leader sequence of Tissue Plasminogen Activator (TPA)
MDAMKRGLCCVLLLCGAVFVSP

SEQ ID NO: 10 - protein, 20 aa, Artificial Sequence
  Native ActRIIA leader
MGAAAKLAFAVFLISCSSGA

SEQ ID NO: 11 - protein, 9 aa, Artificial Sequence
  ActRIIA-hFc and mActRIIA-Fc N-terminal sequence
ILGRSETQE

SEQ ID NO: 12 - protein, 329 aa, Artificial Sequence
  ActRIIA-Fc Protein with deletion of the C-terminal 15 amino acids of the extracellular domain of ActRIIA
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 369 aa, Artificial Sequence
  Unprocessed ActRIIA-hFc with TPA leader sequence
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1113 bp, Artificial Sequence
  Nucleic acid sequence encoding Unprocessed ActRIIA-hFc with TPA leader sequence
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccggtaaatgagaattc

SEQ ID NO: 15 - protein, 106 aa, Artificial Sequence
  human ActRIIB soluble (extracellular processed polypeptide with the N-terminal 6 aa of the EC domain deleted and the C-terminal 4 aa of the EC domain deleted (aa 25-130 of SEQ ID NO28) and with an L79D mutation
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPP

SEQ ID NO: 16 - protein, 512 aa, Homo sapiens
  human ActRIIB precursor protein sequence (A64)
MTAPWVALALLWGSLWPGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 17 - protein, 116 aa, Homo sapiens
  human ActRIIB soluble (extracellular), processed polypeptide sequence (amino acids 19-134 of SEQ ID NO16)
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 18 - protein, 101 aa, Artificial Sequence
  human ActRIIB soluble (extracellular) processed polypeptide with the C-terminal 15 aa deleted (aa 19-119 of SEQ ID NO16)
SGRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 19 - DNA, 1539 bp, Homo sapiens
  nucleic acid sequence encoding a human ActRIIB (A64) precursor protein
atgacggcgccctgggtggccctcgccctcctctggggatcgctgtggcccggctctgggcgtggggaggctgagacacgggagtgcatctactacaacgccaactgggagctggagcgcaccaaccagagcggcctggagcgctgcgaaggcgagcaggacaagcggctgcactgctacgcctcctgggccaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatctaa

SEQ ID NO: 20 - protein, 344 aa, Artificial Sequence
  fusion protein comprising a soluble extracellular domain of ActRIIB (A64; SEQ ID NO17) fused to an Fc domain
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 329 aa, Artificial Sequence
  fusion protein comprising a soluble extracellular domain of ActRIIB (A64) with the C-terminal 15 amino acids deleted (SEQ ID NO18) fused to an Fc domain
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 105 aa, Artificial Sequence
  human ActRIIB soluble (extracellular) processed polypeptide with the N-terminal 6 aa of the EC domain deleted and the C-terminal 5 aa of the EC domain deleted (aa 25-129 of SEQ ID NO28) and with an L79D mutation
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPP

SEQ ID NO: 23 - protein, 107 aa, Artificial Sequence
  human ActRIIB soluble (extracellular) processed polypeptide with the N-terminal 6 aa of the EC domain deleted and the C-terminal 3 aa of the EC domain deleted (aa 25-131 of SEQ ID NO28) and with an L79D mutation
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 24 - protein, 360 aa, Artificial Sequence
  Unprocessed ActRIIB-Fc fusion protein with the N-terminal 6 aa of the EC domain deleted and the C-terminal 3 aa of the EC domain deleted (aa 25-131 of SEQ ID NO28) and with an L79D mutation and with TPA leader sequence
MDAMKRGLCCVLLLCGAVFVSPGAAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 335 aa, Artificial Sequence
  Processed ActRIIB-Fc fusion protein with the N-terminal 6 aa of the EC domain deleted and the C-terminal 3 aa of the EC domain deleted (aa 25-131 of SEQ ID NO28) and with an L79D mutation
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 115 aa, Homo sapiens
  human ActRIIB soluble (extracellular), processed polypeptide sequence (amino acids 20-134 of SEQ ID NO16)
GRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 27 - protein, 100 aa, Artificial Sequence
  human ActRIIB soluble (extracellular), processed polypeptide sequence with the C-terminal 15 aa deleted (aa 20-119 of SEQ ID NO16)
GRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 28 - protein, 512 aa, Homo sapiens
  human ActRIIB precursor protein sequence (R64)
MTAPWVALALLWGSLWPGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 29 - protein, 116 aa, Artificial Sequence
  human ActRIIB soluble (extracellular), processed polypeptide sequence (amino acids 19-134 of SEQ ID NO28)
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 30 - protein, 101 aa, Artificial Sequence
  human ActRIIB soluble (extracellular), processed polypeptide sequence with the C-terminal 15 aa deleted (aa 19-119 of SEQ ID NO28)
SGRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 31 - protein, 115 aa, Artificial Sequence
  human ActRIIB soluble (extracellular), processed polypeptide sequence (amino acids 20-134 of SEQ ID NO28)
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 32 - protein, 100 aa, Artificial Sequence
  human ActRIIB soluble (extracellular), processed polypeptide sequence with the C-terminal 15 aa deleted (aa 20-119 of SEQ ID NO28)
GRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 33 - protein, 107 aa, Artificial Sequence
  human ActRIIB soluble (extracellular) processed polypeptide with the N-terminal 6 aa of the EC domain deleted and the C-terminal 3 aa of the EC domain deleted (aa 25-131 of SEQ ID NO16) and with an L79D mutation
ETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPT

SEQ ID NO: 34 - protein, 360 aa, Artificial Sequence
  Unprocessed ActRIIB-Fc fusion protein with the N-terminal 6 aa of the EC domain deleted and the C-terminal 3 aa of the EC domain deleted (aa 25-131 of SEQ ID NO16) and with an L79D mutation and with TPA leader sequence
MDAMKRGLCCVLLLCGAVFVSPGAAETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 335 aa, Artificial Sequence
  Processed ActRIIB-Fc fusion protein with the N-terminal 6 aa of the EC domain deleted and the C-terminal 3 aa of the EC domain deleted (aa 25-131 of SEQ ID NO16) and with an L79D mutation
ETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 115 aa, Artificial Sequence
  human ActRIIB soluble (extracellular), processed polypeptide sequence (amino acids 20-134 of SEQ ID NO28) with L79D mutation
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPT

SEQ ID NO: 37 - protein, 115 aa, Artificial Sequence
  human ActRIIB soluble (extracellular), processed polypeptide sequence (amino acids 20-134 of SEQ ID NO16) with L79D mutation
GRGEAETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPTAPT

SEQ ID NO: 38 - protein, 343 aa, Artificial Sequence
  human ActRIIB soluble (extracellular), processed polypeptide sequence (aa 20-134 of SEQ ID NO28) with L79D mutation fused to an Fc domain with a GGG linker
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 343 aa, Artificial Sequence
  human ActRIIB soluble (extracellular), processed polypeptide sequence (aa 20-134 of SEQ ID NO16) with L79D mutation fused to an Fc domain
GRGEAETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 368 aa, Artificial Sequence
  human ActRIIB soluble (extracellular), processed polypeptide sequence (aa 20-134 of SEQ ID NO28) with L79D mutation fused to an Fc domain and with TPA leader sequence
MDAMKRGLCCVLLLCGAVFVSPGASGRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 368 aa, Artificial Sequence
  human ActRIIB soluble (extracellular), processed polypeptide sequence (aa 20-134 of SEQ ID NO16) with L79D mutation fused to an Fc domain and with TPA leader sequence
MDAMKRGLCCVLLLCGAVFVSPGASGRGEAETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 141 aa, Artificial Sequence
  human ActRIIB soluble (extracellular), processed polypeptide sequence having a variant C-terminal sequence (disclosed in WO2007/053775)
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEGPWASTTIPSGGPEATAAAGDQGSGALWLCLEGPAHE

SEQ ID NO: 43 - protein, 141 aa, Artificial Sequence
  human ActRIIB soluble (extracellular), processed polypeptide sequence having a variant C-terminal sequence (disclosed in WO2007/053775) having an L79D mutation
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEGPWASTTIPSGGPEATAAAGDQGSGALWLCLEGPAHE

SEQ ID NO: 44 - protein, 370 aa, Artificial Sequence
  human ActRIIB soluble (extracellular), processed polypeptide sequence having a variant C-terminal sequence (disclosed in WO2007/053775) having an L79D mutation fused to an Fc domain with a TGGG linker
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEGPWASTTIPSGGPEATAAAGDQGSGALWLCLEGPAHETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 1083 bp, Artificial Sequence
  Nucleic Acid Sequence Encoding SEQ ID NO24
atggatgcaatgaagagagggctctgctgtgtgctgctgctgtgtggagcagtcttcgtttcgcccggcgccgccgaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctgggacgacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 46 - protein, 344 aa, Artificial Sequence
  fusion protein comprising a soluble extracellular domain of ActRIIB (R64; SEQ ID NO29) fused to an Fc domain
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 329 aa, Artificial Sequence
  fusion protein comprising a soluble extracellular domain of ActRIIB (R64) with the C-terminal 15 aa deleted (SEQ ID NO30) fused to an Fc domain
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 141 aa, Artificial Sequence
  Exemplary human hemoglobin alpha subunit
VLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 49 - protein, 146 aa, Artificial Sequence
  Exemplary human hemoglobin beta subunit
GHFTEEDKATITSLWGKVNVEDAGGETLGRLLVVYPWTQRFFDSFGNLSSASAIMGNPKVKAHGKKVLTSLGDATKHLDDLKGTFAQLSELHCDKLHVDPENFKLLGNVLVTVLAIHFGKEFTPEVQASWQKMVTAVASALSSRYH

SEQ ID NO: 50 - protein, 146 aa, Artificial Sequence
  Exemplary human hemoglobin gamma subunit
VHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

(19) United States
(12) Patent Application Publication
(10) Pub. No.: US 2018/0125928 A1
(43) Pub. Date: May 10, 2018

(54) TREATMENT OF BETA-THALASSEMIA USING ACTRII LIGAND TRAPS

(71) Applicants: CELGENE CORPORATION, Summit, NJ (US); ACCELERON PHARMA INC., Cambridge, MA (US)

(72) Inventors: Kenneth M. Attie, Boston, MA (US); Abderrahmane Laadem, Belle Mead, NJ (US); Rajesh Chopra, Sutton (GB); Jay Backstrom, Leawood, KS (US)

(73) Assignees: CELGENE CORPORATION, Summit, NJ (US); ACCELERON PHARMA INC., Cambridge, MA (US)

(21) Appl. No.: 15/573,177

(22) PCT Filed: May 12, 2016
(86) PCT No.: PCT/US16/31999

§ 371 Date: Nov. 10, 2017

Related U.S. Application Data
(60) Provisional application No. 62/161,136, filed on May 13, 2015.
(60) Provisional application No. 62/173,836, filed on Jun. 10, 2015.
(60) Provisional application No. 62/243,457, filed on Oct. 19, 2015.

(51) Int. Cl. A61K 38/17; A61K 9/00; A61P 7/00; A61P 7/06

(52) U.S. Cl. CPC A61K 38/179; A61P 7/06; A61P 7/00; A61K 9/0019

(57) ABSTRACT

Provided herein are methods of treating beta-thalassemia by subcutaneous administration of about 0.8 mg/kg of an ActRII signaling inhibitor. Also provided herein are methods of adjusting the dose of the ActRII signaling inhibitor administered to the subject.

### 1. CROSS-REFERENCE TO RELATED APPLICATIONS

This application claims the benefit of priority of U.S. Provisional Patent Application No. 62/161,136, filed May 13, 2015, U.S. Provisional Patent Application No. 62/173,836, filed Jun. 10, 2015, and U.S. Provisional Patent Application No. 62/243,457, filed Oct. 19, 2015, the entire contents of each of which are incorporated herein by reference and for all purposes.

### 2. SEQUENCE LISTING

The present application is being filed with a Sequence Listing submitted as file name “12827_952_228_SeqListing.txt”, of size 97 kilobytes, which was created on May 4, 2016. The Sequence Listing is incorporated herein by reference in its entirety and for all purposes.

### 3. FIELD

Provided herein are methods of treating and/or preventing beta-thalassemia, such as transfusion-dependent and non-transfusion-dependent beta-thalassemia, comprising administering to the subject an activin type II receptor signaling inhibitor (ActRII signaling inhibitor, e.g., an activin ligand trap).

### 4. BACKGROUND

Beta-thalassemia, one of the most common inherited hemoglobinopathies worldwide, is due to autosomal mutations in the gene encoding β-globin which induce an absence or low-level synthesis of this protein in erythropoietic cells (Weatherall D J, 2001, Nature Reviews Genetics; 2(4):245-255). About 80 to 90 million people (˜1.5% of the global population) are carriers of □-thalassemia with approximately 60,000 symptomatic individuals born annually (Modell et al., 2007, Scand J Clin Lab Invest; 67:39-69). The annual incidence of symptomatic individuals is estimated at 1 in 100,000 worldwide and 1 in 10,000 in the European Union (EU) (Galanello R and Origa R, 2010, Orphanet J Rare Dis; 5:11). Incidence is highest in the Mediterranean region, the Middle East, and South East Asia (particularly India, Thailand and Indonesia; this region accounts for approximately 50% of affected births) and incidence is increasing worldwide (eg, Europe, the Americas and Australia) as a result of migration (Colah R, Gorakshakar et al., 2010; Expert Rev Hematol; 3(1):103-17; Modell et al., 2008, Bull World Health Organ; 86(6):480-7).

Beta-thalassemias are characterized by a reduction of β-globin chains and a subsequent imbalance in globin chains (α:non-α ratio) of the hemoglobin (Hb) molecule, which results in impaired erythropoiesis and other complications. Nearly 200 different mutations have been described in patients with □-thalassemia that affect the β-globin gene, for which patients may be either homozygous or compound heterozygous. Phenotypic effects, therefore, range widely in patients from slight impairment to complete inhibition of β-globin chain synthesis (Thein S L, 2013, Cold Spring Harb Perspect Med; 3(5):a011700). In addition to deficient β-globin chains, patients may also present with β-thalassemia combined with structural variants such as HbE, leading to HbE/β-thalassemia.

Given the current lack of safe and effective drug therapies to treat beta-thalassemia, for example, transfusion-dependent and non-transfusion-dependent beta-thalassemia, there is significant unmet medical need for the development of new therapies that specifically address the underlying pathophysiology of beta-thalassemia syndromes including anemia and complications of ineffective erythropoiesis.

Two related type II receptors, ActRIIA and ActRIIB, have been identified as the type II receptors for activins (Mathews and Vale, 1991, Cell 65:973-982; Attisano et al., 1992, Cell 68: 97-108). Besides activins, ActRIIA and ActRIIB can biochemically interact with several other TGF-beta family proteins, including BMP7, Nodal, GDF8, and GDF11 (Yamashita et al., 1995, J. Cell Biol. 130:217-226; Lee and McPherron, 2001, Proc. Natl. Acad. Sci. 98:9306-9311; Yeo and Whitman, 2001, Mol. Cell 7: 949-957; Oh et al., 2002, Genes Dev. 16:2749-54). ALK4 is the primary type I receptor for activins, particularly for activin A, and ALK-7 may serve as a receptor for activins as well, particularly for activin B.

An activin ligand trap, consisting of a humanized fusion-protein consisting of the extracellular domain of activin-receptor type IIB (ActRIIB) and the human IgG1 Fc (ActRIIB-hFc), is currently being evaluated in phase II clinical trials for treatment of subjects with beta-thalassemia.

### 5. SUMMARY

Provided herein is a method for treating beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject every 21 days.

Provided herein is a method for treating transfusion-dependent beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject every 21 days.

Provided herein is a method for treating non-transfusion-dependent beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject every 21 days.

Provided herein is a method for treating beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject every 21 days, wherein the genotype of the subject is selected from the group consisting of β⁰/β⁰, β⁺/β⁺, β⁰/β⁺, β⁰/HbE, and β⁺/HbE.

Provided herein is a method for treating beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject every 21 days, wherein the genotype of the subject comprises coinheritance of two severe hemoglobin beta chain mutations, and wherein the subject has alpha-thalassemia.

Provided herein is a method for treating beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject, wherein the genotype of the subject comprises coinheritance of two severe hemoglobin beta chain mutations, and wherein the subject has hereditary persistence of fetal hemoglobin.

Provided herein is a method for treating beta-thalassemia in a subject in need thereof, comprising administering to the subject an initial dose of about 0.8 mg/kg or about 1.0 mg/kg of an activin receptor type II (ActRII) signaling inhibitor, and subsequently administering the ActRII signaling inhibitor to the subject one or more times at 21 day intervals, such that the beta-thalassemia is treated, wherein said administering comprises administering subcutaneously in the upper arm, abdomen, or thigh of the subject.

Provided herein is a method for treating beta-thalassemia in a subject in need thereof, comprising administering to the subject an initial dose of about 0.8 mg/kg or about 1.0 mg/kg of an activin receptor type II (ActRII) signaling inhibitor, and subsequently administering the ActRII signaling inhibitor to the subject one or more times at 21 day intervals, such that the beta-thalassemia is treated, wherein said administering comprises administering subcutaneously in the upper arm, abdomen, or thigh of the subject, and wherein the genotype of the subject is selected from the group consisting of β⁰/β⁰, β⁺/β⁺, β⁰/β⁺, β⁰/HbE, and β⁺/HbE.

Provided herein is a method for treating beta-thalassemia in a subject in need thereof, comprising administering to the subject an initial dose of 0.8 mg/kg or about 1.0 mg/kg of an activin receptor type II (ActRII) signaling inhibitor, and subsequently administering the ActRII signaling inhibitor one or more times at 21 day intervals, such that the beta-thalassemia is treated, wherein said administering comprises administering subcutaneously in the upper arm, abdomen, or thigh of the subject, and wherein the subject has hereditary persistence of fetal hemoglobin.

Provided herein is a method for treating beta-thalassemia in a subject in need thereof, comprising administering to the subject an initial dose of about 0.8 mg/kg or about 1.0 mg/kg of an activin receptor type II (ActRII) signaling inhibitor, and subsequently administering the ActRII signaling inhibitor to the subject one or more times at 21 day intervals, such that the beta-thalassemia is treated, wherein said administering comprises administering subcutaneously in the upper arm, abdomen, or thigh of the subject, and wherein said administering is sufficient to detectably reduce GDF-11 levels in serum from said subject between administrations.

In certain embodiments of any of the foregoing methods, the beta-thalassemia is transfusion-dependent beta-thalassemia. In certain embodiments of any of the foregoing methods, the beta-thalassemia is non-transfusion-dependent beta-thalassemia

In certain embodiments of any of the foregoing methods, the method further comprises taking a first measurement of hemoglobin concentration in the subject; after a first period of time taking a second measurement of hemoglobin concentration in the subject; and administering a subsequent dose of the ActRII signaling inhibitor based on the difference between the second measurement of hemoglobin concentration and the first measurement of hemoglobin concentration, wherein said administering comprises administering subcutaneously in the upper arm, abdomen, or thigh or the subject.

In certain embodiments of any of the foregoing methods, the method further comprises taking a first measurement of hematocrit in the subject; after a first period of time taking a second measurement of hematocrit in the subject; and administering a subsequent dose of the ActRII signaling inhibitor based on the difference between the second measurement of hematocrit and the first measurement of hematocrit, wherein said administering comprises administering subcutaneously in the upper arm, abdomen, or thigh or the subject.

In certain embodiments of any of the foregoing methods, the method further comprises taking a first measurement of fetal hemoglobin in the subject; after a first period of time taking a second measurement of fetal hemoglobin concentration in the subject; and administering a subsequent dose of the ActRII signaling inhibitor based on the difference between the second measurement of fetal hemoglobin concentration and the first measurement of fetal hemoglobin concentration, wherein said administering comprises administering subcutaneously in the upper arm, abdomen, or thigh or the subject.

In certain embodiments of any of the foregoing methods, the method further comprises (a) taking a first measurement of hemoglobin concentration, hematocrit, or fetal hemoglobin concentration in the subject; (b) after a first period of time taking a second measurement of hemoglobin concentration, hematocrit, or fetal hemoglobin concentration in the subject; and (c) after a second period of time, discontinuing administration of the initial dose and administering to the subject a subsequent dose of the ActRII signaling inhibitor, wherein the subsequent dose is administered via subcutaneous injection in the upper arm, abdomen or thigh of the subject.

In certain embodiments of any of the foregoing methods, the first measurement of hemoglobin concentration, hematocrit, or fetal hemoglobin concentration is taken prior to administering to the subject the initial dose the ActRII signaling inhibitor. In certain embodiments, the first measurement of hemoglobin concentration, hematocrit, or fetal hemoglobin concentration is taken immediately after the initial dose the ActRII signaling inhibitor is administered to the subject or within at most 1 day, 2 days, 3 days, 4 days, 5 days, 6 days, or 1 week thereof. In certain embodiments, the second measurement of hemoglobin, hematocrit, or fetal hemoglobin concentration is taken approximately 3 weeks, 1 month, 2 months, 3 months, 4 months, 5 months, 6 months, 7 months, 8 months, 9 months, 10 months, 11 months, or 12 months after the initial dose the ActRII signaling inhibitor is administered to the subject. In certain embodiments, the second period of time is within 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, or 12 weeks of the when the second measurement is taken. In certain embodiments, the subsequent dose of the ActRII signaling inhibitor is about 0.3 mg/kg, about 0.45 mg/kg, about 0.6 mg/kg, about 1.0 mg/kg, or about 1.25 mg/kg. In certain embodiments, the method further comprises taking a third measurement of hemoglobin concentration, hematocrit, or fetal hemoglobin concentration in the subject.

In certain embodiments of any of the foregoing methods, (a) the second measurement of hemoglobin concentration is less than or equal to 12.5 g/dL; (b) the second measurement of hemoglobin concentration is less than or equal to 1.5 g/dL greater than the first measurement of hemoglobin concentration; and (c) the subsequent dose is equal to the initial dose.

In certain embodiments of any of the foregoing methods, (a) the second measurement of hemoglobin concentration is less than or equal to 12.5 g/dL; (b) the second measurement of hemoglobin concentration is greater than 1.5 g/dL greater than the first measurement of hemoglobin concentration; and (c) the subsequent dose is approximately 25% less than the initial dose.

In certain embodiments of any of the foregoing methods, (a) the second measurement of hemoglobin concentration is (i) greater than 12.5 g/dL and less than or equal to 14 g/dL; and (ii) less than or equal to 1.5 g/dL greater than the first measurement of hemoglobin concentration; (b) the subsequent dose is equal to the initial dose; and (c) the second period of time consists of a dose delay of up to twelve weeks until a third measurement of hemoglobin concentration is less than or equal to 12.5 g/dL.

In certain embodiments of any of the foregoing methods, (a) the second measurement of hemoglobin concentration is (i) greater than 12.5 g/dL and less than or equal to 14 g/dL, and (ii) greater than 1.5 g/dL greater than the first measurement of hemoglobin concentration; (b) the subsequent dose is approximately 25% less than the initial dose; and (c) the second period of time consists of a dose delay of up to twelve weeks until a third measurement of hemoglobin concentration is determined to be (i) less than or equal to 12.5 g/dL, and (ii) the change between the first measurement of hemoglobin concentration and the third measurement of hemoglobin concentration is less than or equal to 1.5 g/dL.

In certain embodiments of any of the foregoing methods, (a) the second measurement of hemoglobin concentration is greater than 14 g/dL; (b) the subsequent dose is approximately 25% less than the initial dose; and (c) the second period of time consists of a dose delay of up to twelve weeks until a third measurement of hemoglobin concentration is less than 12.5 g/dL.

In certain embodiments of any of the foregoing methods, the initial dose is administered once every 21 days. In certain embodiments, the subsequent dose is administered once every 21 days.

In certain embodiments of any of the foregoing methods, the method further comprises decreasing GDF11 levels in the subject. In certain embodiments of any of the foregoing methods, the method further comprises increasing fetal hemoglobin levels in the subject.

In certain embodiments of any of the foregoing methods, the ActRII signaling inhibitor is an inhibitor of ActRIIA signaling. In certain embodiments, the ActRII signaling inhibitor is a humanized fusion-protein consisting of the extracellular domain of ActRIIA and the human IgG1 Fc domain. In certain embodiments, ActRIIA signaling inhibitor is a polypeptide comprising an amino acid sequence selected from the group consisting of: (a) 90% identical to SEQ ID NO:2; (b) 95% identical to SEQ ID NO:2; (c) 98% identical to SEQ ID NO:2; (d) SEQ ID NO:2; (e) 90% identical to SEQ ID NO:3; (f) 95% identical to SEQ ID NO:3; (g) 98% identical to SEQ ID NO:3; (h) SEQ ID NO:3; (i) 90% identical to SEQ ID NO:6; (j) 95% identical to SEQ ID NO:6; (k) 98% identical to SEQ ID NO:6; (1) SEQ ID NO:6; (m) 90% identical to SEQ ID NO:7; (n) 95% identical to SEQ ID NO:7; (o) 98% identical to SEQ ID NO:7; and (p) SEQ ID NO:7. In certain embodiments, the ActRII signaling inhibitor is a polypeptide comprising the amino acid sequence of SEQ ID NO:7.

In certain embodiments of any of the foregoing methods, the ActRII signaling inhibitor is an inhibitor of ActRIIB signaling. In certain embodiments, the ActRII signaling inhibitor is a humanized fusion-protein consisting of the extracellular domain of ActRIIB and the human IgG1 Fc domain. In certain embodiments, the ActRIIB inhibitor is a polypeptide comprising an amino acid sequence selected from the group consisting of: (a) 90% identical to SEQ ID NO:17; (b) 95% identical to SEQ ID NO:17; (c) 98% identical to SEQ ID NO:17; (d) SEQ ID NO:17; (e) 90% identical to SEQ ID NO:20; (f) 95% identical to SEQ ID NO:20; (g) 98% identical to SEQ ID NO:20; (h) SEQ ID NO:20; (i) 90% identical to SEQ ID NO:21; (j) 95% identical to SEQ ID NO:21; (k) 98% identical to SEQ ID NO:21; (1) SEQ ID NO:21; (m) 90% identical to SEQ ID NO:25; (n) 95% identical to SEQ ID NO:25; (o) 98% identical to SEQ ID NO:25; and (p) SEQ ID NO:25. In certain embodiments, the ActRIIB signaling inhibitor is a polypeptide comprising the amino acid sequence of SEQ ID NO:25.

Provided herein is a method for treating beta-thalassemia in a subject in need thereof, comprising administering to the subject an initial dose of about 0.8 mg/kg of an activin receptor type IIB (ActRIIB) signaling inhibitor, wherein the activin receptor type IIB (ActRIIB) signaling inhibitor is administered subcutaneously in the upper arm, abdomen, or thigh of the subject every 21 days, and wherein the ActRIIB signaling inhibitor comprises the amino acid sequence of SEQ ID NO:25.

Provided herein is a method for treating transfusion-dependent beta-thalassemia in a subject in need thereof, comprising administering to the subject an initial dose of about 0.8 mg/kg of an activin receptor type IIB (ActRIIB) signaling inhibitor, wherein the activin receptor type II (ActRIIB) signaling inhibitor is administered subcutaneously in the upper arm, abdomen, or thigh of the subject every 21 days, and wherein the ActRIIB signaling inhibitor comprises the amino acid sequence of SEQ ID NO:25.

Provided herein is a method for treating non-transfusion-dependent beta-thalassemia in a subject in need thereof, comprising administering to the subject an initial dose of about 0.8 mg/kg of an activin receptor type IIB (ActRIIB) signaling inhibitor, wherein the activin receptor type IIB (ActRIIB) signaling inhibitor is administered subcutaneously in the upper arm, abdomen, or thigh of the subject every 21 days, and wherein the ActRIIB signaling inhibitor comprises the amino acid sequence of SEQ ID NO:25.

Provided herein is a method for treating beta-thalassemia in a subject in need thereof, comprising administering to the subject an initial dose of about 0.8 mg/kg of an activin receptor type IIB (ActRIIB) signaling inhibitor, wherein the activin receptor type IIB (ActRIIB) signaling inhibitor is administered subcutaneously in the upper arm, abdomen, or thigh of the subject every 21 days, wherein the genotype of the subject is selected from the group consisting of β⁰/β⁰, β⁺43⁺, β⁰/β⁺, β⁰/HbE, and β⁺/HbE, and wherein the ActRIIB signaling inhibitor comprises the amino acid sequence of SEQ ID NO:25.

Provided herein is a method for treating beta-thalassemia in a subject in need thereof, comprising administering to the subject an initial dose of about 0.8 mg/kg or about 1.0 mg/kg of an activin receptor type IIB (ActRIIB) signaling inhibitor, wherein the activin receptor type IIB (ActRIIB) signaling inhibitor is administered subcutaneously in the upper arm, abdomen, or thigh of the subject every 21 days, wherein the genotype of the subject comprises coinheritance of two severe hemoglobin beta chain mutations, wherein the subject has alpha-thalassemia, and wherein the ActRIIB signaling inhibitor comprises the amino acid sequence of SEQ ID NO:25.

Provided herein is a method for treating beta-thalassemia in a subject in need thereof, comprising administering to the subject an initial dose of about 0.8 mg/kg or about 1.0 mg/kg of an activin receptor type IIB (ActRIIB) signaling inhibitor, wherein the activin receptor type IIB (ActRIIB) signaling inhibitor is administered subcutaneously in the upper arm, abdomen, or thigh of the subject every 21 days, wherein the genotype of the subject comprises coinheritance of two severe hemoglobin beta chain mutations, wherein the subject has hereditary persistence of fetal hemoglobin, and wherein the ActRIIB signaling inhibitor comprises the amino acid sequence of SEQ ID NO:25.

Provided herein is a method for treating beta-thalassemia in a subject in need thereof, comprising administering to the subject an initial dose of about 0.8 mg/kg or about 1.0 mg/kg of an activin receptor type IIB (ActRIIB) signaling inhibitor, and subsequently administering the ActRIIB signaling inhibitor to the subject one or more times at 21 day intervals, such that the beta-thalassemia is treated, wherein said administering comprises administering subcutaneously in the upper arm, abdomen, or thigh of the subject.

Provided herein is a method for treating beta-thalassemia in a subject in need thereof, comprising administering to the subject an initial dose of about 0.8 mg/kg or about 1.0 mg/kg of an activin receptor type IIB (ActRIIB) signaling inhibitor, and subsequently administering the ActRIIB signaling inhibitor to the subject one or more times at 21 day intervals, such that the beta-thalassemia is treated, wherein said administering comprises administering subcutaneously in the upper arm, abdomen, or thigh of the subject, and wherein the genotype of the subject is selected from the group consisting of β⁰/β⁰, β⁺/β⁺, β⁰/β⁺, β⁰/HbE, and β⁺/HbE.

Provided herein is a method for treating beta-thalassemia in a subject in need thereof, comprising administering to the subject an initial dose of about 0.8 mg/kg or about 1.0 mg/kg of an activin receptor type IIB (ActRIIB) signaling inhibitor, and subsequently administering the ActRIIB signaling inhibitor one or more times at 21 day intervals, such that the beta-thalassemia is treated, wherein said administering comprises administering subcutaneously in the upper arm, abdomen, or thigh of the subject, and wherein the subject has hereditary persistence of fetal hemoglobin.

Provided herein is a method for treating beta-thalassemia in a subject in need thereof, comprising administering to the subject an initial dose of about 0.8 mg/kg or about 1.0 mg/kg of an activin receptor type IIB (ActRIIB) signaling inhibitor, and subsequently administering the ActRIIB signaling inhibitor to the subject one or more times at 21 day intervals, such that the beta-thalassemia is treated, wherein said administering comprises administering subcutaneously in the upper arm, abdomen, or thigh of the subject, and wherein said administering is sufficient to detectably reduce GDF-11 levels in serum from said subject between administrations.

In certain embodiments of any of the foregoing methods, the beta-thalassemia is transfusion-dependent beta-thalassemia. In certain embodiments of any of the foregoing methods, the beta-thalassemia is non-transfusion-dependent beta-thalassemia.

In certain embodiments of any of the foregoing methods, the method further comprises taking a first measurement of hemoglobin concentration in the subject; after a first period of time taking a second measurement of hemoglobin concentration in the subject; and administering a subsequent dose of the ActRIIB signaling inhibitor based on the difference between the second measurement of hemoglobin concentration and the first measurement of hemoglobin concentration, wherein said administering comprises administering subcutaneously in the upper arm, abdomen, or thigh or the subject.

In certain embodiments of any of the foregoing methods, the method further comprises taking a first measurement of hematocrit in the subject; after a first period of time taking a second measurement of hematocrit in the subject; and administering a subsequent dose of the ActRIIB signaling inhibitor based on the difference between the second measurement of hematocrit and the first measurement of hematocrit, wherein said administering comprises administering subcutaneously in the upper arm, abdomen, or thigh or the subject.

In certain embodiments of any of the foregoing methods, the method further comprises taking a first measurement of fetal hemoglobin concentration in the subject; after a first period of time taking a second measurement of fetal hemoglobin concentration in the subject; and administering a subsequent dose of the ActRIIB signaling inhibitor based on the difference between the second measurement of fetal hemoglobin concentration and the first measurement of fetal hemoglobin concentration, wherein said administering comprises administering subcutaneously in the upper arm, abdomen, or thigh or the subject.

In certain embodiments of any of the foregoing methods, the method further comprises (a) taking a first measurement of hemoglobin concentration in the subject; (b) after a first period of time taking a second measurement of hemoglobin concentration in the subject; and (c) after a second period of time, discontinuing administration of the initial dose and administering to the subject a subsequent dose of the ActRIIB signaling inhibitor, wherein the subsequent dose is administered via subcutaneous injection in the upper arm, abdomen or thigh of the subject.

In certain embodiments of any of the foregoing methods, the first measurement of hemoglobin concentration, hematocrit, or fetal hemoglobin concentration is taken prior to administering to the subject the initial dose the ActRIIB signaling inhibitor. In certain embodiments, the first measurement of hemoglobin concentration, hematocrit, or fetal hemoglobin concentration is immediately after the initial dose the ActRIIB signaling inhibitor is administered to the subject or within at most 1 day, 2 days, 3 days, 4 days, 5 days, 6 days, or 1 week thereof. In certain embodiments, the second measurement of hemoglobin concentration, hematocrit, or fetal hemoglobin concentration is taken approximately 3 weeks, 1 month, 2 months, 3 months, 4 months, 5 months, 6 months, 7 months, 8 months, 9 months, 10 months, 11 months, or 12 months after the initial dose the ActRIIB signaling inhibitor is administered to the subject. In certain embodiments, the second period of time is within 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, or 12 weeks of the when the second measurement is taken. In certain embodiments, the subsequent dose of the ActRIIB signaling inhibitor is about 0.3 mg/kg, about 0.45 mg/kg, about 0.6 mg/kg, about 1.0 mg/kg, or about 1.25 mg/kg. In certain embodiments, the method further comprises taking a third measurement of hemoglobin concentration, hematocrit, or fetal hemoglobin concentration in the subject.

In certain embodiments of any of the foregoing methods, (a) the second measurement of hemoglobin concentration is less than or equal to 12.5 g/dL; (b) the second measurement of hemoglobin concentration is less than or equal to 1.5 g/dL greater than the first measurement of hemoglobin concentration; and (c) the subsequent dose is equal to the initial dose.

In certain embodiments of any of the foregoing methods, (a) the second measurement of hemoglobin concentration is less than or equal to 12.5 g/dL; (b) the second measurement of hemoglobin concentration is greater than 1.5 g/dL greater than the first measurement of hemoglobin concentration; and (c) the subsequent dose is approximately 25% less than the initial dose.

In certain embodiments of any of the foregoing methods, (a) the second measurement of hemoglobin concentration is (i) greater than 12.5 g/dL and less than or equal to 14 g/dL; and (ii) less than or equal to 1.5 g/dL greater than the first measurement of hemoglobin concentration; (b) the subsequent dose is equal to the initial dose; and (c) the second period of time consists of a dose delay of up to twelve weeks until a third measurement of hemoglobin concentration is less than or equal to 12.5 g/dL.

In certain embodiments of any of the foregoing methods, (a) the second measurement of hemoglobin concentration is (i) greater than 12.5 g/dL and less than or equal to 14 g/dL, and (ii) greater than 1.5 g/dL greater than the first measurement of hemoglobin concentration; (b) the subsequent dose is approximately 25% less than the initial dose; and (c) the second period of time consists of a dose delay of up to twelve weeks until a third measurement of hemoglobin concentration is determined to be (i) less than or equal to 12.5 g/dL, and (ii) the change between the first measurement of hemoglobin concentration and the third measurement of hemoglobin concentration is less than or equal to 1.5 g/dL.

In certain embodiments of any of the foregoing methods, (a) the second measurement of hemoglobin concentration is greater than 14 g/dL; (b) the subsequent dose is approximately 25% less than the initial dose; and (c) the second period of time consists of a dose delay of up to twelve weeks until a third measurement of hemoglobin concentration is less than 12.5 g/dL.

In certain embodiments of any of the foregoing methods, the initial dose is administered once every 21 days. In certain embodiments of any of the foregoing methods, the subsequent dose is administered once every 21 days.

In certain embodiments of any of the foregoing methods, the method further comprises decreasing GDF11 levels in the subject.

In certain embodiments of any of the foregoing methods, the method further comprises increasing fetal hemoglobin levels in the subject.

Provided herein is a method of increasing fetal hemoglobin levels in a subject comprising administering an ActRIIB signaling inhibitor to the subject.

In certain embodiments of any of the foregoing methods, the subject expresses hemoglobin E.

In certain embodiments of any of the foregoing methods, the subject does not express hemoglobin S.

In certain embodiments of any of the foregoing methods, the erythroid response consists of (i) a greater than or equal to 33% reduction in transfusion burden for 12 weeks, and (ii) a reduction of at least 2 units of red blood cells over a 12 week period.

In certain embodiments of any of the foregoing methods, the erythroid response consists of a greater than 1 g/dL increase in hemoglobin concentration as compared to a baseline hemoglobin concentration, wherein the increase in hemoglobin concentration is measured by the mean of hemoglobin concentration values over a contiguous 12-week period in the absence of transfusion.

In certain embodiments of any of the foregoing methods, the subject is a human.

In certain embodiments of any of the foregoing methods, the ActRII signaling inhibitor is packaged in a container as a sterile, preservative-free lyophilized cake, stored between 2° C. and 8° C. prior to administration to the subject. In certain embodiments, the container contains 37.5 mg of the ActRII signaling inhibitor. In certain embodiments, the container contains 75 mg of the ActRII signaling inhibitor.

### 6. BRIEF DESCRIPTION OF THE FIGURES

FIG. 1 depicts the healing of a leg ulcer in an exemplary transfusion dependent patient prior to treatment and after receiving ActRIIB-hFc (SEQ ID NO:25) at a dose of 1.25 mg/kg for 2 weeks or 5 weeks.

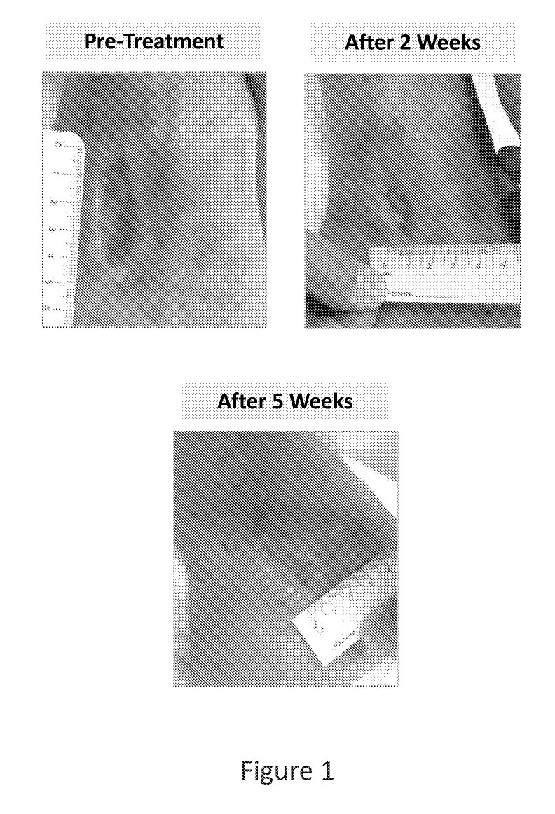

### 7. DETAILED DESCRIPTION

### 7.1 Overview

Provided herein are methods of treating beta-thalassemia, such as transfusion-dependent or non-transfusion-dependent beta-thalassemia, in a subject comprising administering to the subject an ActRII signaling inhibitor.

### 7.2 Abbreviations and Terminology

As used herein, the term “about” when used in conjunction with a number refers to any number within 1%, 2%, 3%, 4%, 5%, 6%, 7%, 8%, 9%, 10%, 11%, 12%, 13%, 14%, or 15% of the referenced number. In certain embodiments, the term “about” encompasses the exact number recited.

As used herein, “ActRII” refers to activin receptor type II. As used herein, “ActRIIA” refers to activin receptor type IIA. See, for example, Mathews and Vale, 1991, Cell 65:973-982. GenBank™ accession number NM_001278579.1 provides an exemplary human ActRIIA nucleic acid sequence. GenBank™ accession number NP_001265508.1 provides an exemplary human ActRIIA amino acid sequence. As used herein, “ActRIIB” refers to activin receptor type IIB. See, for example, Attisano et al., 1992, Cell 68: 97-108. GenBank™ accession number NM_001106.3 provides an exemplary human ActRIIB nucleic acid sequence. GenBank™ accession number NP_001097.2 provides an exemplary human ActRIIB amino acid sequence.

As used herein, “ActRIIA-mFc” or “mActRIIA-Fc” refers to a mouse activin type IIA receptor-IgG1 fusion protein. See, for example, U.S. Pat. No. 8,173,601. As used herein, “mActRIIB-Fc” or “ActRIIB-mFc” refers to a mouse activin type IIB receptor-IgG1 fusion protein. See, for example, U.S. Pat. No. 8,173,601. As used herein, “hActRIIA-Fc” or “ActRIIA-hFc” refers to a human activin type IIA receptor-IgG1 fusion protein. See, for example, U.S. Pat. No. 8,173,601. In certain embodiments, ActRIIA-hFc refers to a polypeptide comprising the amino acid sequence of SEQ ID NO: 7. As used herein, “hActRIIB-Fc” or “ActRIIB-hFc” refers to a human activin type IIB receptor-IgG1 fusion protein. See, for example, U.S. Pat. No. 8,173,601. In certain embodiments, ActRIIB-hFc refers to a polypeptide comprising the amino acid sequence of SEQ ID NO: 25.

“AE” refers to adverse events.

“β⁰” refers to an allele associated with a lack of beta globin subunit synthesis.

“β⁺” refers to an allele associated with reduced beta globin subunit synthesis.

“Hb” refers to hemoglobin protein. GenBank™ Accession No. NP_000549.1 (SEQ ID NO:48) provides an exemplary amino acid sequence of a human hemoglobin alpha subunit. GenBank™ Accession No. NP_000509.1 (SEQ ID NO:49) provides an exemplary amino acid sequence of a human hemoglobin beta subunit. GenBank™ Accession No. NP_000550.2 (SEQ ID NO:50) provides an exemplary amino acid sequence of a human hemoglobin gamma subunit. Typically, the most common form of hemoglobin in a human adult comprises two alpha subunits and two beta subunits. Fetal hemoglobin, also referred to as “hemoglobin F” or “HbF” comprises two alpha subunits and two gamma subunits.

“HbE” or “Hemoglobin E” is an art recognized term and refers to a mutated form of hemoglobin, for example, human hemoglobin. Hemoglobin E comprises two alpha subunits and two beta subunits, wherein position 26 of the beta subunit is mutated from glutamic acid to lysine (E26K).

“HbE/beta-thalassemia” refers to the co-inheritance of hemoglobin E and a β⁰ allele.

“HbS” or “Hemoglobin S” is an art recognized term and refers to a mutated form of hemoglobin, for example, human hemoglobin. Hemoglobin S comprises two alpha subunits and two beta subunits, wherein position 6 of the beta subunit is mutated from glutamine to valine (G6V).

In certain embodiments, one unit of red blood cells refers to a quantity of packed red blood cells derived from approximately 400-500 mL of donated blood.

### 7.3 Methods of Treatment and/or Prevention

### 7.3.1 Beta-Thalassemia

In certain embodiments, provided herein are methods for treating and/or preventing beta-thalassemia in a subject, comprising administering to the subject an initial dose of about 0.5 mg/kg, about 0.6 mg/kg, about 0.7 mg/kg, about 0.8 mg/kg, about 0.9 mg/kg, about 1.0 mg/kg, or about 1.1 mg/kg of an ActRII signaling inhibitor (e.g., an activin ligand trap), wherein the ActRII signaling inhibitor is administered to the subject subcutaneously in the upper arm, abdomen, or thigh of the subject.

In certain embodiments, provided herein are methods for treating and/or preventing beta-thalassemia in a subject, comprising administering to the subject an initial dose of about 0.8 mg/kg of an ActRII signaling inhibitor (e.g., an activin ligand trap), wherein the ActRII signaling inhibitor is administered to the subject subcutaneously in the upper arm, abdomen, or thigh of the subject.

In certain embodiments, “treat,” “treatment,” or “treating,” in the context of beta-thalassemia, includes amelioration of at least one symptom of beta-thalassemia. Nonlimiting examples of symptoms of beta thalassemia include defective red blood cell production in the marrow, ineffective erythropoiesis, deficient hemoglobin levels, multiple organ dysfunction, iron overload, paleness, fatigue, jaundice, and splenomegaly.

In certain embodiments, the subject is a subject as described in Section 7.5. In certain embodiments, the beta-thalassemia is transfusion-dependent beta-thalassemia. In certain embodiments, the beta-thalassemia is non-transfusion-dependent beta-thalassemia.

In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25). In certain embodiments, the ActRII signaling inhibitor is an ActRIIA signaling inhibitor as described in Section 7.6.1. In certain embodiments, the ActRIIA signaling inhibitor is an ActRIIA-Fc such as an ActRIIA-hFc (e.g., SEQ ID NO:7).

In certain embodiments, the ActRII signaling inhibitor is administered to the subject as a composition as described in Section 7.9.

In certain embodiments, the ActRII signaling inhibitor is administered to the subject in combination with a second pharmaceutically active agent or therapy as described in Section 7.8.

In certain embodiments, the method further comprises administering to the subject a subsequent dose of the ActRII signaling inhibitor as described in Section 7.3.2 or Section 7.4. For example, the method can further comprise analysis of hemoglobin concentration in the subject as a means to determine a subsequent dosing regimen to be administered to the subject. In certain embodiments, hemoglobin concentration in the subject may be used (i) to evaluate appropriate dosing for a subject, wherein the subject is a candidate to be treated or is being treated with an ActRII signaling inhibitor (e.g., an activin ligand trap); (ii) to evaluate whether to adjust the dosage of the ActRII signaling inhibitor during treatment; and/or (iii) to evaluate an appropriate maintenance dose of the ActRII signaling inhibitor. Depending on the hemoglobin concentration in the subject, dosing with an ActRII signaling inhibitor may be initiated, increased, reduced, delayed or terminated. See, for example, Table 1 and Table 2. In certain embodiments, the method further comprises (a) taking a first measurement of hemoglobin concentration in the subject; (b) after a first period of time taking a second measurement of hemoglobin concentration in the subject; and (c) after a second period of time, discontinuing administration of the initial dose and administering to the subject a subsequent dose of the ActRII signaling inhibitor, wherein the subsequent dose is administered via subcutaneous injection in the upper arm, abdomen or thigh of the subject. In certain embodiments, the method further comprises taking a third measurement of hemoglobin concentration in the subject. In certain embodiments, the subsequent dose of the ActRII signaling inhibitor is titrated up to a maximum subsequent dose of about 1.25 mg/kg. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in an erythroid response in the subject. In certain embodiments, the erythroid response comprises a reduction in transfusion burden in the subject by at least 33%, wherein the subject has transfusion-dependent beta thalassemia. In certain embodiments, the erythroid response comprises a reduction in transfusion burden in the subject by at least 50%, wherein the subject has transfusion-dependent beta thalassemia. In certain embodiments, the erythroid response comprises a reduction in transfusion burden in the subject by at least 25%, 30%, 33%, 35%, 40%, 45%, 50%, 55%, 60%, 65%, 70%, 75%, 80%, 85%, 90%, 95%, 98%, or 100%, wherein the subject has transfusion-dependent beta thalassemia. In certain embodiments, the erythroid response comprises a reduction in transfusion burden in the subject by at least 25%, 30%, 33%, 35%, 40%, 45%, 50%, 55%, 60%, 65%, 70%, 75%, 80%, 85%, 90%, 95%, 98%, or 100% for at least 8 weeks, 9 weeks, 10 weeks, 11 weeks, 12 weeks, 13 weeks, 14 weeks, 15 weeks, 16 weeks, 5 months, 6 months, 7 months, 8 months, 9 months, 10 months, 11 months, or 12 months, wherein the subject has transfusion-dependent beta thalassemia. In certain embodiments, the erythroid response comprises a reduction in transfusion burden in the subject by at least 33% for at least 12 weeks, wherein the subject has transfusion-dependent beta thalassemia. In certain embodiments, the erythroid response comprises a reduction in transfusion burden in the subject by at least 50% for at least 12 weeks, wherein the subject has transfusion-dependent beta thalassemia. In certain embodiments, the erythroid response comprises a reduction of red blood cell transfusion in the subject by at least 1, 2, 3, 4, or more red blood cells units, wherein the subject has transfusion-dependent beta thalassemia. In certain embodiments, the erythroid response comprises a reduction of red blood cell transfusion in the subject by at least 1, 2, 3, 4, or more red blood cell units for at least 8 weeks, 9 weeks, 10 weeks, 11 weeks, 12 weeks, 13 weeks, 14 weeks, 15 weeks, 16 weeks, 5 months, 6 months, 7 months, 8 months, 9 months, 10 months, 11 months, or 12 months least. In certain embodiments, the erythroid response comprises a reduction of at least two units of red blood cells for at least 12 weeks in the subject, wherein the subject has transfusion-dependent beta thalassemia. In certain embodiments, the erythroid response comprises (i) a reduction in transfusion burden in the subject by at least 33% for at least 12 weeks, and (ii) a reduction of at least two units of red blood cells for at least 12 weeks in the subject, wherein the subject has transfusion-dependent beta thalassemia. In certain embodiments, the reduction in transfusion burden is as compared to the transfusion burden for the subject within 1 week, 2 weeks, 3 weeks, or 4 weeks prior to the commencement of treatment of the subject according to the methods provided herein at baseline. In certain embodiments, the reduction in units of red blood cells is as compared to the units of red blood cells administered to the subject the subject within 1 week, 2 weeks, 3 weeks, or 4 weeks prior to the commencement of treatment of the subject according to the methods provided herein. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in an erythroid response in the subject. In certain embodiments, the erythroid response comprises an increase in the hemoglobin concentration in the subject by greater than 0.75 g/dL, 1 g/dL, 1.25 g/dL, or 1.5 g/dL as compared to the hemoglobin concentration in the subject prior to treatment according to the methods provided herein, wherein the hemoglobin concentration is measured by the mean of hemoglobin concentrations in the subject over at least a contiguous 12-week period in the absence of transfusion in the subject, and wherein the subject has non-transfusion-dependent beta thalassemia. In certain embodiments, the erythroid response comprises an increase in the hemoglobin concentration in the subject by greater than 1 g/dL as compared to the hemoglobin concentration in the subject prior to treatment according to the methods provided herein, wherein the hemoglobin concentration is measured by the mean of hemoglobin concentrations in the subject over at least a contiguous 12-week period in the absence of transfusion in the subject, and wherein the subject has non-transfusion-dependent beta thalassemia. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, a transfusion-dependent beta-thalassemia subject treated according to the methods provided herein does not require red blood cell transfusion for at least 8 weeks, 9 weeks, 10 weeks, 12 weeks, 4 months, 5 months, 6 months, 7 months, 8 months, 9 months, 10 months, 11 months, or 1 year after treatment. In certain embodiments, a transfusion-dependent beta-thalassemia subject treated according to the methods provided herein does not require red blood cell transfusion for at least 8 weeks after treatment. In certain embodiments, a transfusion-dependent beta-thalassemia subject treated according to the methods provided herein does not require red blood cell transfusion for at least 12 weeks after treatment. In certain embodiments, a transfusion-dependent beta-thalassemia subject treated according to the methods provided herein does not require red blood cell transfusion for at least 8 weeks after treatment. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in a decrease in liver iron concentration in the subject by at least 5%, 10%, 15%, 20%, 25%, 30%, 35%, 40%, 45%, 50%, 55%, 60%, 65%, 70%, 75%, 80%, 85%, 90%, 95%, or at least 100%, or by at most 5%, 10%, 15%, 20%, 25%, 30%, 35%, 40%, 45%, 50%, 55%, 60%, 65%, 70%, 75%, 80%, 85%, 90%, 95%, or at most 100% as compared to the levels of liver iron concentration in the subject within 1 week, 2 weeks, 3 weeks, or 4 weeks prior to the commencement of treatment of the subject according to the methods provided herein. In certain embodiments, the liver iron concentration in the subject decreases by about 10% as compared to the liver iron concentration in the subject within 1 week, 2 weeks, 3 weeks, or 4 weeks prior to the commencement of treatment of the subject according to the methods provided herein. In certain embodiments, the liver iron concentration in the subject decreases by about 15% as compared to the liver iron concentration in the subject within 1 week, 2 weeks, 3 weeks, or 4 weeks prior to the commencement of treatment of the subject according to the methods provided herein. In certain embodiments, the liver iron concentration in the subject decreases by about 20% as compared to the liver iron concentration in the subject within 1 week, 2 weeks, 3 weeks, or 4 weeks prior to the commencement of treatment of the subject according to the methods provided herein. In certain embodiments, the liver iron concentration in the subject decreases by between 5% and 30% as compared to the liver iron concentration in the subject within 1 week, 2 weeks, 3 weeks, or 4 weeks prior to the commencement of treatment of the subject according to the methods provided herein. In certain embodiments, the liver iron concentration in the subject decreases by between 10% and 30% as compared to the liver iron concentration in the subject within 1 week, 2 weeks, 3 weeks, or 4 weeks prior to the commencement of treatment of the subject according to the methods provided herein. In certain embodiments, liver iron concentration is determined according to an assay described in Section 7.7. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in a decrease in myocardial iron concentration in the subject by at least 5%, 10%, 15%, 20%, 25%, 30%, 35%, 40%, 45%, 50%, 55%, 60%, 65%, 70%, 75%, 80%, 85%, 90%, 95%, or at least 100%, or by at most 5%, 10%, 15%, 20%, 25%, 30%, 35%, 40%, 45%, 50%, 55%, 60%, 65%, 70%, 75%, 80%, 85%, 90%, 95%, or at most 100% as compared to myocardial iron concentration in the subject within 1 week, 2 weeks, 3 weeks, or 4 weeks prior to the commencement of treatment of the subject according to the methods provided herein. In certain embodiments, myocardial iron concentration is determined according to an assay described in Section 7.7. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in reduced daily iron chelation therapy in the subject, such as, for example, a decrease in the dose or frequency of one or more iron chelation therapeutic agents administered to the subject. Nonlimiting examples of iron chelation therapeutic agents include deferasirox, deferiprone, and deferoxamine. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in reduced serum ferritin levels in the subject by at least 5%, 10%, 15%, 20%, 25%, 30%, 35%, 40%, 45%, 50%, 55%, 60%, 65%, 70%, 75%, 80%, 85%, 90%, 95%, or at least 100%, or by at most 5%, 10%, 15%, 20%, 25%, 30%, 35%, 40%, 45%, 50%, 55%, 60%, 65%, 70%, 75%, 80%, 85%, 90%, 95%, or at most 100% as compared to the serum ferritin levels in the subject within 1 week, 2 weeks, 3 weeks, or 4 weeks prior to the commencement of treatment of the subject according to the methods provided herein. In certain embodiments, serum ferritin levels are determined according to an assay described in Section 7.7. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in an increase in fetal hemoglobin concentration in the subject by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or at least 500%, or by at most 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or at most 500%, as compared to fetal hemoglobin concentration in the subject within 1, 2, 3, or 4 weeks prior to the commencement of treatment of the subject according to the methods provided herein. In certain embodiments, the fetal hemoglobin concentration is determined according to an assay as described in Section 7.7. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in a decrease in GDF11 concentration in the subject by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or at least 500%, or by at most 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or at most 500%, as compared to GDF11 concentration in the subject within 1, 2, 3, or 4 weeks prior to the commencement of treatment of the subject according to the methods provided herein. In certain embodiments, the GDF11 concentration is determined according to an assay as described in Section 7.7. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein reduces the symptoms associated with one or more beta-thalassemia clinical complications as compared to the symptoms within 1, 2, 3, or 4 weeks prior to treatment of the subject according to the methods provided herein. In certain embodiments, treatment of a subject (e.g., a subject as described in Section 7.5) according to the methods provided herein reduces the symptoms associated with one or more transfusion-dependent beta-thalassemia clinical complications. Non-limiting examples of transfusion-dependent beta-thalassemia include growth retardation, pallor, jaundice, poor musculature, genu valgum, hepatosplenomegaly, leg ulcers, development of masses from extramedullary hematopoiesis, skeletal changes resulting from expansion of the bone marrow, and clinical complications of chronic red blood cell transfusions, such as, for example hepatitis B virus infection, hepatitis C virus infection, and human immunodeficiency virus infection, alloimmunization, and organ damage due to iron overload, such as, for example, liver damage, heart damage, and endocrine gland damage. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein reduces the symptoms associated with one or more non-transfusion-dependent beta-thalassemia clinical complications as compared to the symptoms in the subject within 1, 2, 3, or 4 weeks prior to the commencement of treatment of the subject according to the methods provided herein. Non-limiting examples of non-transfusion-dependent beta-thalassemia include endocrine abnormalities, such as, for example, diabetes mellitus, hypothyroidism, hypogonadism, thrombotic events, pulmonary hypertension, hypercoagulability, the development of transfusion-dependency later in life, ineffective erythropoiesis, expansion of the hematopoietic tissue outside of the marrow medulla, formation of extramedullary hematopoiesis masses, skeletal deformities, osteopenia, osteoporosis, bone pain, gallstones, leg ulcers, and alloimmunization. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein improves red blood cell morphology in the subject as compared to the red blood cell morphology in the subject within 1, 2, 3, or 4 weeks prior to the commencement of treatment of the subject according to the methods provided herein. Nonlimiting determinants of improved red blood cell morphology include a reduction in the ratio of number of abnormal red blood cells in the subject to the total number of red blood cells in the subject, a reduction in the ratio of the number of red blood cells with basophilic stippling in the subject to the total number of red blood cells in the subject, a reduction in the ratio of the number of poikilocytic red blood cells in the subject to the total number of red blood cells in the subject, a reduction in the ratio of the number of schistocytes in the subject to the total number of red blood cells in the subject, and a reduction in the ratio of the number of irregularly contracted red blood cells in the subject to the total number of red blood cells in the subject. In certain embodiments, treatment of a subject (e.g., a subject as described in Section 7.5) according to the methods provided herein results in a reduction in the ratio of the number of abnormal red blood cells in the subject to the total number of red blood cells in the subject in the subject by at least 5%, 10%, 15%, 20%, 25%, 30%, 35%, 40%, 45%, 50%, 55%, 60%, 65%, 70%, 75%, 80%, 85%, 90%, 95%, or at least 100%, or by at most 5%, 10%, 15%, 20%, 25%, 30%, 35%, 40%, 45%, 50%, 55%, 60%, 65%, 70%, 75%, 80%, 85%, 90%, 95%, or at most 100% as compared to the ratio of the number of abnormal red blood cells in the subject to the total number of red blood cells in the subject within 1, 2, 3, or 4 weeks prior to the commencement of treatment of the subject according to the methods provided herein. In certain embodiments, treatment of a subject (e.g., a subject as described in Section 7.5) according to the methods provided herein results in a reduction in the ratio of the number of red blood cells with basophilic stippling in the subject to the total number of red blood cells in the subject in the subject by at least 5%, 10%, 15%, 20%, 25%, 30%, 35%, 40%, 45%, 50%, 55%, 60%, 65%, 70%, 75%, 80%, 85%, 90%, 95%, or at least 100%, or by at most 5%, 10%, 15%, 20%, 25%, 30%, 35%, 40%, 45%, 50%, 55%, 60%, 65%, 70%, 75%, 80%, 85%, 90%, 95%, or at most 100% as compared to the ratio of the number of red blood cells with basophilic stippling in the subject to the total number of red blood cells in the subject within 1, 2, 3, or 4 weeks prior to the commencement of treatment of the subject according to the methods provided herein. In certain embodiments, treatment of a subject (e.g., a subject as described in Section 7.5) according to the methods provided herein results in a reduction in the ratio of the number of poikilocytic red blood cells in the subject to the total number of red blood cells in the subject by at least 5%, 10%, 15%, 20%, 25%, 30%, 35%, 40%, 45%, 50%, 55%, 60%, 65%, 70%, 75%, 80%, 85%, 90%, 95%, or at least 100%, or by at most 5%, 10%, 15%, 20%, 25%, 30%, 35%, 40%, 45%, 50%, 55%, 60%, 65%, 70%, 75%, 80%, 85%, 90%, 95%, or at most 100% as compared to the ratio of the number of poikilocytic red blood cells in the subject to the total number of red blood cells in the subject within 1, 2, 3, or 4 weeks prior to the commencement of treatment of the subject according to the methods provided herein. In certain embodiments, treatment of a subject (e.g., a subject as described in Section 7.5) according to the methods provided herein results in a reduction in the ratio of the number of schistocytes in the subject to the total number of red blood cells in the subject by at least 5%, 10%, 15%, 20%, 25%, 30%, 35%, 40%, 45%, 50%, 55%, 60%, 65%, 70%, 75%, 80%, 85%, 90%, 95%, or at least 100%, or by at most 5%, 10%, 15%, 20%, 25%, 30%, 35%, 40%, 45%, 50%, 55%, 60%, 65%, 70%, 75%, 80%, 85%, 90%, 95%, or at most 100% as compared to the ratio of the number of schistocytes in the subject to the total number of red blood cells in the subject within 1, 2, 3, or 4 weeks prior to the commencement of treatment of the subject according to the methods provided herein. In certain embodiments, treatment of a subject (e.g., a subject as described in Section 7.5) according to the methods provided herein results in a reduction in the ratio of the number of irregularly contracted red blood cells in the subject to the total number of red blood cells in the subject by at least 5%, 10%, 15%, 20%, 25%, 30%, 35%, 40%, 45%, 50%, 55%, 60%, 65%, 70%, 75%, 80%, 85%, 90%, 95%, or at least 100%, or by at most 5%, 10%, 15%, 20%, 25%, 30%, 35%, 40%, 45%, 50%, 55%, 60%, 65%, 70%, 75%, 80%, 85%, 90%, 95%, or at most 100% as compared to the ratio of the number of irregularly contracted red blood cells in the subject to the total number of red blood cells in the subject within 1, 2, 3, or 4 weeks prior to the commencement of treatment of the subject according to the methods provided herein. In certain embodiments, the red blood cell morphology is determined according to an assay as described in Section 7.7. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in the reduction of 1, 2, 3, 4, or more symptoms of osteoporosis within 1, 2, 3, or 4 weeks prior to the commencement of treatment of the subject according to the methods provided herein. In certain embodiments, treatment of a subject (e.g., a subject as described in Section 7.5) according to the methods provided herein results in the reduction of 1, 2, 3, 4, or more symptoms of osteopenia within 1, 2, 3, or 4 weeks prior to the commencement of treatment of the subject according to the methods provided herein. In certain embodiments, treatment of a subject (e.g., a subject as described in Section 7.5) according to the methods provided herein results in an increase in the bone mineral density in the subject by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or at least 500%, or by at most 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or at most 500%, as compared to the bone mineral density in the subject within 1, 2, 3, or 4 weeks prior to the commencement of treatment of the subject according to the methods provided herein. In certain embodiments, the bone mineral density is the total body bone mineral density, the total hip bone mineral density, or the lumbar spine bone mineral density. In certain embodiments, the bone mineral density is determined according to an assay as described in Section 7.7. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in a decrease in skeletal deformities in the subject as compared to skeletal deformities in the subject within 1, 2, 3, or 4 weeks prior to the commencement of treatment of the subject according to the methods provided herein. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, treatment of a subject (e.g., a subject as described in Section 7.5) according to the methods provided herein results in an improvement in the quality of life in the subject as compared to the quality of life in the subject within 1, 2, 3, or 4 weeks prior to the commencement of treatment of the subject according to the methods provided herein. In certain embodiments, the quality of life is determined according to an assay as described in Section 7.7. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

7.3.2 Adjusted Dosing

Also provided herein are methods of treating beta-thalassemia in subject in need thereof (see, Section 7.3.1), further comprising analysis of hemoglobin concentration in the subject as a means to determine a subsequent dosing regimen to be administered to the subject. In certain embodiments, hemoglobin concentration in the subject may be used (i) to evaluate appropriate dosing for a subject, wherein the subject is a candidate to be treated or is being treated with an ActRII signaling inhibitor (e.g., an activin ligand trap); (ii) to evaluate whether to adjust the dosage of the ActRII signaling inhibitor during treatment; and/or (iii) to evaluate an appropriate maintenance dose of the ActRII signaling inhibitor. Depending on the hemoglobin concentration in the subject, dosing with an ActRII signaling inhibitor may be initiated, increased, reduced, delayed or terminated. See, for example, Table 1 and Table 2. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

TABLE 1
Subsequent Dosing Regimens: Dose delay, dose reduction, and dose discontinuation

| Event | Action |
|---|---|
| Any related adverse event ≥ Grade 2^{e} | Dose delay^{a} |
| | Hold until resolved < Grade 2 or baseline |
| Any related adverse event ≥ Grade 3 | Dose delay^{a} |
| | Hold until resolved < Grade 2 or baseline and |
| | Dose reduce with 25% |

If Hb ≤12.5 g/dL^{b} and:

| | |
|---|---|
| Δ Hb ≤1.5^{c} g/dL at Day 21 from Last Dose | Continue dosing following schedule at the |
| | same Dose Level. |
| Δ Hb >1.5^{c} g/dL at Day 21 from Last Dose | Reduce dose level by 25%. |

If Hb >12.5 g/dL^{b, d} ≤14 g/dL^{b, d} and

| | |
|---|---|
| Δ Hb ≤1.5^{c} g/dL at Day 21 from Last Dos | Dose delay for up to an additional 12 weeks |
| | until Hb ≤12.5 g/dL. Continue dosing at the |
| | same Dose Level. |
| Δ Hb >1.5^{c} g/dL at Day 21 from Last Dose | Dose delay for up to an additional 12 weeks |
| | until Hb ≤12.5 g/dL, Δ Hb ≤1.5 g/dL. Reduce |
| | dose level by 25%. |
| If Hb >14 g/dL | Dose delay for up to an additional 12 weeks |
| | until Hb <12.5 g/dl and Dose reduced 25% |
| If a subject experiences ≥2 Dose Reductions | Discontinue treatment |
| due to related adverse event | |

^{a}Dose delay of ActRII signaling inhibitor is defined as a dose not administered >4 days from the planned dosing date due to Hb >12.5 g/dL and/or ActRII signaling inhibitor-related toxicity ≥ Grade 2.
^{b}Based on the pre-transfusion/pre-treatment Hb value at the time of re-treatment.
^{c}Hemoglobin non influence by a transfusion, i.e., Hb ≥1421 days post transfusion
^{d}If Hb >12.5 g/dL Hb measurement should occur every week. If dosing of a subject is delayed for more than 12 weeks (up to a maximum of 15 weeks delay from the previous dose administered), the treatment should be discontinued.
^{e}For a description of Grade 2 and Grade 3 scoring of adverse events, see Section 7.7.14.

TABLE 2
Starting dose level with dose reductions and escalation

| Subsequent Dose | | | | Initial | Subsequent Dose | |
|---|---|---|---|---|---|---|
| 4^{th} Dose | 3^{rd} Dose | 2^{nd} Dose | 1^{st} Dose | Dose | 1^{st} Dose | 2^{nd} Dose |
| Reduc- | Reduc- | Reduc- | Reduc- | Initial | Escala- | Escala- |
| tion | tion | tion | tion | Dose | tion | tion |
| Discon- | About | About | About | About | About | About |
| tinue | 0.3 | 0.45 | 0.6 | 0.8 | 1.0 | 1.25 |
| treat- | mg/kg | mg/kg | mg/kg | mg/kg | mg/kg | mg/kg |
| ment | | | | | | |

In certain embodiments, the method of treating beta-thalassemia in subject in need thereof (see, Section 7.3.1), further comprises (a) taking a first measurement of hemoglobin concentration in the subject; (b) after a first period of time taking a second measurement of hemoglobin concentration in the subject; and (c) after a second period of time, discontinuing administration of the initial dose and administering to the subject a subsequent dose of the ActRII signaling inhibitor, wherein the subsequent dose is administered via subcutaneous injection in the upper arm, abdomen or thigh of the subject. In certain embodiments, the method further comprises taking a third measurement of hemoglobin concentration in the subject. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, the first measurement and/or the second measurement is taken as described in Section 7.7. In certain embodiments, the first measurement of hemoglobin concentration is taken prior to administering to the subject the initial dose the ActRII signaling inhibitor. In certain embodiments, the first measurement of hemoglobin concentration is immediately after the initial dose the ActRII signaling inhibitor is administered to the subject or within at most 1 day, 2 days, 3 days, 4 days, 5 days, 6 days, or 1 week thereof. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, the second measurement of hemoglobin concentration is taken approximately 3 weeks, 1 month, 2 months, 3 months, 4 months, 5 months, 6 months, 7 months, 8 months, 9 months, 10 months, 11 months, or 12 months after the initial dose the ActRII signaling inhibitor is administered to the subject.

In certain embodiments, the second period of time is within 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, or 12 weeks of the when the second measurement is taken.

In certain embodiments, the subsequent dose of the ActRII signaling inhibitor is about 0.3 mg/kg, about 0.45 mg/kg, about 0.6 mg/kg, about 1.0 mg/kg, or about 1.25 mg/kg. In certain embodiments, the subsequent dose of the ActRII signaling inhibitor is titrated up to a maximum subsequent dose of about 1.25 mg/kg. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, the method further comprises taking a third measurement of hemoglobin concentration in the subject.

In a specific embodiment, (a) the second measurement of hemoglobin concentration is less than or equal to 12.5 g/dL; (b) the second measurement of hemoglobin concentration is less than or equal to 1.5 g/dL greater than the first measurement of hemoglobin concentration; and (c) the subsequent dose is equal to the initial dose. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In another specific embodiment, (a) the second measurement of hemoglobin concentration is less than or equal to 12.5 g/dL; (b) the second measurement of hemoglobin concentration is greater than 1.5 g/dL greater than the first measurement of hemoglobin concentration; and (c) the subsequent dose is approximately 25% less than the initial dose. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In yet another specific embodiment, (a) the second measurement of hemoglobin concentration is (i) greater than 12.5 g/dL and less than or equal to 14 g/dL; and (ii) less than or equal to 1.5 g/dL greater than the first measurement of hemoglobin concentration; (b) the subsequent dose is equal to the initial dose; and (c) the second period of time consists of a dose delay of up to twelve weeks until a third measurement of hemoglobin concentration is less than or equal to 12.5 g/dL. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In yet another specific embodiment, (a) the second measurement of hemoglobin concentration is (i) greater than 12.5 g/dL and less than or equal to 14 g/dL, and (ii) greater than 1.5 g/dL greater than the first measurement of hemoglobin concentration; (b) the subsequent dose is approximately 25% less than the initial dose; and (c) the second period of time consists of a dose delay of up to twelve weeks until a third measurement of hemoglobin concentration is determined to be (i) less than or equal to 12.5 g/dL, and (ii) the change between the first measurement of hemoglobin concentration and the third measurement of hemoglobin concentration is less than or equal to 1.5 g/dL. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In yet another specific embodiment, (a) the second measurement of hemoglobin concentration is greater than 14 g/dL; (b) the subsequent dose is approximately 25% less than the initial dose; and (c) the second period of time consists of a dose delay of up to twelve weeks until a third measurement of hemoglobin concentration is less than 12.5 g/dL. In certain embodiments, the method further comprises determining a third measurement of hemoglobin concentration. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, the initial dose is administered as described in Section 7.4. In certain embodiments, the initial dose is administered to the subject once every 14, 15, 16, 17, 18, 19, 20, 21, 22, 23, 24, 25, 26, 27, or 28 days. In certain embodiments, the initial dose is administered to the subject via subcutaneous injection. In certain embodiments, the initial dose is administered to the subject in the upper arm, abdomen, or thigh of the subject. In certain embodiments, the initial dose is administered to the subject once every 14, 15, 16, 17, 18, 19, 20, 21, 22, 23, 24, 25, 26, 27, or 28 days, via subcutaneous injection in the upper arm, abdomen, or thigh of the subject. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, the initial dose is administered as described in Section 7.4. In certain embodiments, the initial dose is administered to the subject once every 21 days. In certain embodiments, the initial dose is administered to the subject via subcutaneous injection. In certain embodiments, the initial dose is administered to the subject in the upper arm, abdomen, or thigh of the subject. In certain embodiments, the initial dose is administered to the subject once every 21 days, via subcutaneous injection in the upper arm, abdomen, or thigh of the subject. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, the initial dose is administered as described in Section 7.4. In certain embodiments, the initial dose is administered to the subject once every 14, 15, 16, 17, 18, 19, 20, 21, 22, 23, 24, 25, 26, 27, or 28 days. In certain embodiments, the initial dose is administered to the subject via subcutaneous injection. In certain embodiments, the initial dose is administered to the subject in the upper arm, abdomen, or thigh of the subject. In certain embodiments, the initial dose is administered to the subject once every 14, 15, 16, 17, 18, 19, 20, 21, 22, 23, 24, 25, 26, 27, or 28 days, via subcutaneous injection in the upper arm, abdomen, or thigh of the subject. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, the subsequent dose is administered as described in Section 7.4. In certain embodiments, the subsequent dose is administered to the subject once every 21 days. In certain embodiments, the subsequent dose is administered to the subject via subcutaneous injection. In certain embodiments, the subsequent dose is administered to the subject in the upper arm, abdomen, or thigh of the subject. In certain embodiments, the subsequent dose is administered to the subject once every 21 days, via subcutaneous injection in the upper arm, abdomen, or thigh of the subject. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain embodiments, the subsequent dose is administered as described in Section 7.4. In certain embodiments, the subsequent dose is administered to the subject once every 14, 15, 16, 17, 18, 19, 20, 21, 22, 23, 24, 25, 26, 27, or 28 days. In certain embodiments, the subsequent dose is administered to the subject via subcutaneous injection. In certain embodiments, the subsequent dose is administered to the subject in the upper arm, abdomen, or thigh of the subject. In certain embodiments, the subsequent dose is administered to the subject once every 14, 15, 16, 17, 18, 19, 20, 21, 22, 23, 24, 25, 26, 27, or 28 days, via subcutaneous injection in the upper arm, abdomen, or thigh of the subject. In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRIIB signaling inhibitor is an ActRIIB-Fc such as an ActRIIB-hFc (e.g., SEQ ID NO:25).

In certain other embodiments, the subject is a subject as described in Section 7.5. In certain embodiments, the subject has beta-thalassemia. In certain embodiments, the subject has transfusion-dependent beta-thalassemia. In certain embodiments, the subject has beta-thalassemia major. In certain embodiments, the transfusion-dependent beta-thalassemia is beta-thalassemia major. In certain embodiments, the subject has non-transfusion-dependent beta-thalassemia. In certain embodiments, the subject has beta-thalassemia intermediate. In certain embodiments, the non-transfusion-dependent beta-thalassemia is beta-thalassemia intermediate.

In certain embodiments, the hemoglobin concentration (i.e., the first hemoglobin concentration, the second hemoglobin concentration, and the third hemoglobin concentration) is determined as described in Section 7.7.

In certain embodiments, the methods provided herein are utilized in combination with a second pharmaceutically active agent or therapy, as described in Section 7.8.

In certain embodiments, the ActRII signaling inhibitor is as described in Section 7.6. In certain embodiments, the ActRII signaling inhibitor is an ActRIIB signaling inhibitor as described in Section 7.6.2. In certain embodiments, the ActRII signaling inhibitor is ActRIIB-Fc such as ActRIIB-hFc (e.g., SEQ ID NO:25). In certain embodiments, the ActRII signaling inhibitor is an ActRIIA signaling inhibitor as described in Section 7.6.1. In certain embodiments, the ActRII signaling inhibitor is ActRIIA-Fc such as ActRIIA-hFc (e.g., SEQ ID NO:7).

### 7.4 Dosing Regimens

In certain embodiments, the dose of the ActRII signaling inhibitor administered according to the methods provided herein (see Section 7.3.1 and 7.3.2) is about 0.5 mg/kg, about 0.6 mg/kg, about 0.7 mg/kg, about 0.8 mg/kg, about 0.9 mg/kg, about 1.0 mg/kg, about 1.1 mg/kg, or about 1.2 mg/kg. In certain embodiments, the dose of the ActRII signaling inhibitor administered according to the methods provided herein (see Section 7.3.1 and 7.3.2) is about 0.8 mg/kg. In certain embodiments, an ActRII inhibitor is an inhibitor of ActRIIB signaling as set forth in Section 7.6.2. In certain embodiments, the ActRII signaling inhibitor is an ActRIIB-Fc such as an ActRIIB-hFc (e.g., SEQ ID NO:25). In certain embodiments, an ActRII signaling inhibitor is an inhibitor of ActRIIA signaling as set forth in Section 7.6.1. In certain embodiments, the ActRII signaling inhibitor is an ActRIIA-Fc such as an ActRIIA-hFc (e.g., SEQ ID NO:7). In certain embodiments, the ActRII signaling inhibitor is a combination of an ActRIIA signaling inhibitor and an ActRIIB signaling inhibitor.

In certain embodiments, the ActRII signaling inhibitor is administered to the subject subcutaneously. In certain embodiments, the ActRII signaling inhibitor is administered to the subject subcutaneously in the upper arm, abdomen, or thigh of the subject. In certain embodiments, the ActRII signaling inhibitor is administered to the subject every 21 days. In certain embodiments, the ActRII signaling inhibitor is administered to the subject every 14, 15, 16, 17, 18, 19, 20, 21, 22, 23, 24, 25, 26, 27, or 28 days. In certain embodiments, the ActRII signaling inhibitor is administered to the subject every 21 days, subcutaneously in the upper arm, abdomen, or thigh of the subject. In certain embodiments, the ActRII signaling inhibitor is administered to the subject every 14, 15, 16, 17, 18, 19, 20, 21, 22, 23, 24, 25, 26, 27, or 28 days days, subcutaneously in the upper arm, abdomen, or thigh of the subject.

In certain embodiments, the ActRII signaling inhibitor is a composition as described in Section 7.9. In certain embodiments, the ActRII signaling inhibitor is a sterile, preservative-free, lyophilized powder reconstituted in water for injection. In certain embodiments, a single dose of the ActRII signaling inhibitor is reconstituted in a volume of water for injection of greater than 1 mL. In such embodiments, the single dose of the ActRII signaling inhibitor is administered to the subject via two injections of equal volume of reconstituted ActRII signaling inhibitor. In certain embodiments, the two injections are administered to the subject at separate sites, e.g., one injection in the right thigh and one injection in the left thigh.

In certain embodiments, the dose of the ActRII signaling inhibitor is an initial dose. In certain embodiments, the initial dose is about 0.8 mg/kg.

In certain embodiments, the dose of the ActRII signaling inhibitor is a subsequent dose. In certain embodiments, the subsequent dose is greater than the initial dose. In certain embodiments, the subsequent dose is less than the initial dose. In certain embodiments, the subsequent dose is about 0.3 mg/kg, about 0.45 mg/kg, about 0.6 mg/kg, about 1.0 mg/kg, or about 1.25 mg/kg. In certain embodiments, the subsequent dose is about 0.3 mg/kg, about 0.45 mg/kg, about 0.6 mg/kg, about 1.0 mg/kg, or about 1.25 mg/kg. In certain embodiments, the subsequent dose is about 0.3 mg/kg. In certain embodiments, the subsequent dose is about 0.45 mg/kg. In certain embodiments, the subsequent dose is about 0.6 mg/kg. In certain embodiments, the subsequent dose is about 1.0 mg/kg. In certain embodiments, the subsequent dose is about 1.25 mg/kg. In certain embodiments, the subsequent dose is about 2.5 mg, about 5 mg, about 10 mg, about 15 mg, about 20 mg, or about 35 mg greater than the initial dose, or about 0.05 mg/kg, about 0.1 mg/kg, about 0.15 mg/kg, about 0.25 mg/kg, about 0.3 mg/kg, about 0.35 mg/kg, about 0.4 mg/kg, or about 0.5 mg/kg greater than the initial dose.

In certain embodiments, the subsequent dose of the ActRII signaling inhibitor is dosed at intervals and amounts sufficient to achieve serum concentrations of about 0.2 microgram/kg or greater, and serum levels of about 1 microgram/kg or 2 microgram/kg or greater are desirable for achieving significant effects on bone density and strength. Subsequent dosing regimens may be designed to reach serum concentrations of between 0.2 and 15 microgram/kg, and optionally between 1 and 5 microgram/kg. In humans, serum levels of 0.2 microgram/kg may be achieved with a single subsequent dose of about 0.1 mg/kg or greater and serum levels of 1 microgram/kg may be achieved with a single subsequent dose of about 0.3 mg/kg or greater. The observed serum half-life of the molecule is between about 20 and 30 days, substantially longer than most Fc fusion proteins, and thus a sustained effective serum level may be achieved, for example, by dosing with about 0.2-0.4 mg/kg on a weekly or biweekly basis, or higher doses may be used with longer intervals between dosings. For example, subsequent doses of about 1-3 mg/kg might be used on a monthly or bimonthly basis, and the effect on bone may be sufficiently durable that dosing is necessary only once every 3, 4, 5, 6, 9, 12 or more months. Serum levels of the ActRII signaling inhibitor can be measured by any means known to the skilled artisan. For example, antibodies against the ActRII signaling inhibitor can be used to determine the serum levels of the ActRII signaling inhibitor using, e.g., an ELISA.

In certain embodiments, the subsequent dose is administered more frequently than the initial dose. In certain embodiments, the subsequent dose is administered less frequently than the initial dose. In certain embodiments, the subsequent dose is administered at the same frequency as the initial dose. In certain embodiments, the subsequent dose is administered every 14, 15, 16, 17, 18, 19, 20, 21, 22, 23, 24, 25, 26, 27, or 28 days. In certain embodiments, the subsequent dose is administered every 21 days. In certain embodiments, the subsequent dose is administered continuously and/or indefinitely.

When used in conjunction with a dose provided herein (e.g., a dose of an ActRII signaling inhibitor or a dose of a second active agent), the word “about” refers to any number within 1, 5 or 10% of the referenced number.

### 7.5 Patient Populations

The subjects treated in accordance with the methods described herein can be any mammals such as rodents and primates, and in a preferred embodiment, humans. In certain embodiments, the methods described herein can be used to treat beta-thalassemia in a subject, such as, transfusion-dependent beta-thalassemia, non-transfusion-dependent beta-thalassemia, beta-thalassemia major, and beta-thalassemia intermediate, to reduce transfusion burden in a subject with beta-thalassemia, or to monitor said treatment, and/or to select subjects to be treated in accordance with the methods provided herein, in any mammal such as a rodent or primate, and in a preferred embodiment, in a human subject.

In certain embodiments, the subject treated in accordance with the methods described here can be of any age. In certain embodiments, the subject treated in accordance with the methods described herein is less than 18 years old. In a specific embodiment, the subject treated in accordance with the methods described herein is less than 13 years old. In another specific embodiment, the subject treated in accordance with the methods described herein is less than 12, less than 11, less than 10, less than 9, less than 8, less than 7, less than 6, or less than 5 years old. In another specific embodiment, the subject treated in accordance with the methods described herein is 1-3 years old, 3-5 years old, 5-7 years old, 7-9 years old, 9-11 years old, 11-13 years old, 13-15 years old, 15-20 years old, 20-25 years old, 25-30 years old, or greater than 30 years old. In another specific embodiment, the subject treated in accordance with the methods described herein is 30-35 years old, 35-40 years old, 40-45 years old, 45-50 years old, 50-55 years old, 55-60 years old, or greater than 60 years old. In another specific embodiment, the subject treated in accordance with the methods described herein is 60-65 years old, 65-70 years old, 70-75 years old, 75-80 years old, or greater than 80 years old.

In certain embodiments, the subject treated in accordance with the methods described herein (see Section 7.3), has beta-thalassemia. In certain embodiments, the beta-thalassemia is transfusion-dependent beta-thalassemia. Transfusion-dependent beta-thalassemia is also known as “Cooley's anemia”. In certain embodiments, the beta-thalassemia is beta-thalassemia major. In certain embodiments, the transfusion-dependent beta-thalassemia is beta-thalassemia major. In certain embodiments, the beta-thalassemia is non-transfusion-dependent beta-thalassemia. In certain embodiments, the beta-thalassemia is beta-thalassemia intermediate. In certain embodiments, the transfusion-dependent beta-thalassemia is non-beta-thalassemia intermediate. In certain embodiments, the subject has HbE/beta thalassemia. In certain embodiments, the subject (i) has beta-thalassemia major; (ii) has severe HbE/beta-thalassemia; and (iii) is transfusion-dependent. In certain embodiments, the subject (i) has beta-thalassemia intermedia; (ii) has mild/moderate HbE/beta-thalassemia; and (iii) is non-transfusion-dependent.

In certain embodiments, the subject treated in accordance with the methods described herein (see Section 7.3), has transfusion-dependent beta-thalassemia. In certain embodiments, the subject has been diagnosed with transfusion-dependent beta-thalassemia. In certain embodiments, the subject has been diagnosed with beta-thalassemia and hemoglobin E. In certain embodiments, the diagnosis has been confirmed by genetic analysis. In certain embodiments, the transfusion-dependent beta-thalassemia is beta-thalassemia major. In certain embodiments, the transfusion-dependent beta-thalassemia is beta-thalassemia major. In certain embodiments, the subject comprises a genotype comprising homozygosity or compound heterozygosity for a mutant beta globin allele. In certain embodiments, the homozygosity comprises β⁰/β⁰, wherein β⁰ refers to an allele associated with lack of beta globin chain synthesis. In certain embodiments, the homozygosity comprises β⁺/β⁺, wherein β⁺ refers to an allele associated with reduced beta globin chain synthesis. In certain embodiments, the compound heterozygosity comprises β⁰/β⁺, wherein β⁰ refers to an allele associated with lack of beta globin chain synthesis, and wherein β⁺ refers to an allele associated with reduced beta globin chain synthesis. In certain embodiments, the compound heterozygosity comprises β⁰/HbE, wherein β⁰ refers to an allele associated with lack of beta globin chain synthesis, and wherein HbE refers to hemoglobin E. In certain embodiments, the compound heterozygosity comprises β⁺/HbE, wherein β⁺ refers to an allele associated with reduced beta globin chain synthesis, and wherein HbE refers to hemoglobin E. In certain embodiments, the subject has symptomatic thalassemia. In certain embodiments, the subject has co-inherited duplication of the alpha-globin gene. In certain embodiments, the subject has been diagnosed with transfusion-dependent beta-thalassemia. In certain embodiments, the diagnosis has been confirmed by genetic analysis. In certain embodiments, the subject is a human infant subject. In certain embodiments, the subject has hereditary persistence of fetal hemoglobin.

In certain embodiments, the subject requires regular, lifelong red blood cell transfusions. In certain embodiments, a subject having transfusion-dependent beta-thalassemia requires transfusion of more than 5 red blood cell units over a 24 week period. In certain embodiments, a subject having transfusion-dependent beta-thalassemia requires transfusion of more than 6 red blood cell units over a 24 week period. In certain embodiments, the subject has a high transfusion burden. In certain embodiments, high transfusion burden is 12 or more red blood cell units over 24 weeks prior to treatment according to the methods provided herein. In certain embodiments, the subject has a low transfusion burden. In certain embodiments, low transfusion burden is 7-12 red blood cell units over 24 weeks prior to treatment according to the methods provided herein.

In certain embodiments, the subject has one or more transfusion-dependent beta-thalassemia clinical complications. Non-limiting examples of transfusion-dependent beta-thalassemia clinical complications include growth retardation, pallor, jaundice, poor musculature, genu valgum, hepatosplenomegaly, leg ulcers, development of masses from extramedullary hematopoiesis, and skeletal changes resulting from expansion of the bone marrow. In certain embodiments, the subject has one or more complications of chronic red blood cell transfusions. Non-limiting examples of complications of chronic red blood cell transfusions include transfusion-associated infections, such as, for example, hepatitis B virus infection, hepatitis C virus infection, and human immunodeficiency virus infection, alloimmunization, and organ damage due to iron overload, such as, for example, liver damage, heart damage, and endocrine gland damage.

In certain embodiments, the subject treated in accordance with the methods described herein (see Section 7.3), has non-transfusion-dependent beta-thalassemia. In certain embodiments, a subject having non-transfusion-dependent beta-thalassemia requires transfusion of 0 to 5 red blood cell units over a 24 week period. In certain embodiments, a subject having non-transfusion-dependent beta-thalassemia requires transfusion of 0 to 6 red blood cell units over a 24 week period. In certain embodiments, the subject has been diagnosed with beta-thalassemia. In certain embodiments, the subject has been diagnosed with beta-thalassemia and hemoglobin E. In certain embodiments, the beta-thalassemia has been confirmed by genetic analysis. In certain embodiments, the non-transfusion-dependent beta-thalassemia is beta-thalassemia intermedia. In certain embodiments, the non-transfusion-dependent beta thalassemia is mild-moderate hemoglobin E/beta-thalassemia. In certain embodiments, the non-transfusion-dependent beta-thalassemia does not require regular red blood cell transfusion. In certain embodiments, the subject seldom requires red blood cell transfusions. In certain embodiments, the non-transfusion-dependent beta-thalassemia requires regular red blood cell transfusion later in life. In certain embodiments, the subject has received 0 to 5 red blood cell units during the 24-week period prior to treatment according to the methods provided herein. In certain embodiments, the subject has received 0 to 6 red blood cell units during the 24-week period prior to treatment according to the methods provided herein. In certain embodiments, the subject has a mean baseline hemoglobin level of less than 10.0 g/dL.

In certain embodiments, the beta-thalassemia is non-transfusion-dependent beta-thalassemia. In certain embodiments, the beta-thalassemia is beta-thalassemia intermediate. In certain embodiments, the transfusion-dependent beta-thalassemia is non-beta-thalassemia intermediate. In certain embodiments, the subject comprises a genotype comprising compound heterozygosity. In certain embodiments, the compound heterozygosity comprises a β⁰ allele, wherein β⁰ refers to an allele associated with lack of beta globin chain synthesis. In certain embodiments, the compound heterozygosity comprises a β⁺ allele, wherein β⁺ refers to an allele associated with reduced beta globin chain synthesis. In certain embodiments, the compound heterozygosity comprises β⁰/β⁺, wherein β⁰ refers to an allele associated with lack of beta globin chain synthesis, and wherein β⁺ refers to an allele associated with reduced beta globin chain synthesis. In certain embodiments, the compound heterozygosity comprises one or more hemoglobin variants. In certain embodiments, the hemoglobin variant is hemoglobin E. In certain embodiments, the subject (i) comprises a genotype comprising coinheritance of two severe beta globin chain mutations, and (ii) has alpha-thalassemia. In certain embodiments, the subject (i) comprises a genotype comprising coinheritance of two severe beta globin chain mutations, and (ii) has hereditary persistence of fetal hemoglobin. In certain embodiments, the subject has symptomatic thalassemia. In certain embodiments, the subject has co-inherited duplication of the alpha-globin gene. In certain embodiments, the subject has been diagnosed with beta-thalassemia. In certain embodiments, the diagnosis has been confirmed by genetic analysis.

In certain embodiments, the subject displays one or more non-transfusion-dependent beta-thalassemia clinical complications. Non-limiting examples of non-transfusion-dependent beta-thalassemia clinical complications include endocrine abnormalities, such as, for example, diabetes mellitus, hypothyroidism, hypogonadism, thrombotic events, pulmonary hypertension, hypercoagulability, the development of transfusion-dependency later in life, ineffective erythropoiesis, expansion of the hematopoietic tissue outside of the marrow medulla, formation of extramedullary hematopoiesis masses, skeletal deformities, osteopenia, osteoporosis, bone pain, gallstones, and leg ulcers. In certain embodiments, the subject exhibits alloimmunization.

In certain embodiments, the subject displays mild symptoms beta-thalassemia symptoms. In certain embodiments, the subject has near normal growth.

In certain embodiments, the non-transfusion-dependent beta-thalassemia subject displays severe symptoms. Non-limiting examples of severe symptoms include growth retardation, development retardation, and skeletal deformities.

In certain embodiments, the subject has splenomegaly. In certain embodiments, the splenomegaly develops in the first 6-12 months of the subject's life.

In certain embodiments, the subject has impaired growth during the first 10 years of the subject's life.

In certain embodiments, the subject exhibits microcytic, hypochromic anemia. In certain embodiments, the hemoglobin A2 levels in the subject prior to treatment of the subject according to the methods provided herein are elevated as compared to the hemoglobin A2 levels in a reference population (e.g., a reference population as described in Section 7.7). In certain embodiments, the fetal hemoglobin levels in the subject prior to treatment of the subject according to the methods provided herein is elevated as compared to the fetal hemoglobin levels in a reference population (e.g., a reference population as described in Section 7.7).

In certain embodiments, the subject does not express hemoglobin S.

In certain embodiments, the subject does not express hemoglobin S. In certain embodiments, the subject has not received red blood cell transfusions within 12 weeks prior to treatment according to the methods provided herein, wherein the subject has non-transfusion-dependent beta-thalassemia. In certain embodiments, the subject does not have active hepatitis C infection. In certain embodiments, the subject does not have active hepatitis B infection. In certain embodiments, the subject is not positive for human immunodeficiency virus. In certain embodiments, the subject does not have insulin-dependent diabetes. In certain embodiments, the subject has not been administered an erythropoiesis stimulating agent within 3 months prior to treatment according to the methods provided herein. In certain embodiments, the subject has not undergone iron chelation therapy within 168 days prior to treatment according to the methods provided herein. In certain embodiments, the subject has not undergone hydroxyurea treatment within 168 days prior to treatment according to the methods provided herein. In certain embodiments, the subject has not been administered biphosphonates within the 168 days prior to treatment according to the methods provided herein. In certain embodiments, the subject does not have uncontrolled hypertension. Uncontrolled hypertension refers to >Grade 1 according to NCI CTCAE version 4.0. In certain embodiments, the subject does not have liver disease with ALT greater than 3 times the upper limit of normal. In certain embodiments, the subject does not have liver disease with histopathological evidence of liver cirrhosis/fibrosis as determined by liver biopsy. In certain embodiments, the subject does not have heart disease. Heart disease or heart failure can be classified by the New York Heart Association as classification 3 or higher. In certain embodiments, the subject does not have arrhythmia requiring treatment. In certain embodiments, the subject does not have lung disease. Non-limiting examples of lung disease include pulmonary fibrosis and pulmonary hypertension. In certain embodiments the subject does not have a creatinine clearance rate of less than 60 mL/min as determined by the Cockroff-Gault method. In certain embodiments, the subject does not have folate deficiency. In certain embodiments, the subject does not have proteinuria of Grade 3 or higher. In certain embodiments, the subject does not have adrenal insufficiency. In certain embodiments, the subject has not undergone a major surgery within 30 days prior to treatment according to the methods provided herein, except for wherein the major surgery is splenectomy. In certain embodiments, the subject does not have a history of severe allergic or anaphylactic reactions or hypersensitivity to recombinant proteins. In certain embodiments, the subject has not undergone long-term anticoagulant therapy. Nonlimiting examples of anti-coagulant therapy includes heparin and warfarin. In certain embodiments, the subject is not undergoing treatment with cytotoxic agents, systemic corticosteroids, immunosuppressants, or anticoagulant therapy within 28 days prior to treatment according to the methods provided herein.

In certain embodiments, the subject is undergoing other treatment interventions. Non-limiting examples of other treatment interventions include splenectomy, transfusion therapy, iron chelation therapy, and fetal hemoglobin-inducing agents. In certain embodiments, the subject requires iron chelation therapy. See Section 7.8 for a description of combination therapies.

In certain embodiments, the subject is a subject as described in Section 8.

As used herein, the words “patient” and “subject” are used interchangeably.

### 7.6 Inhibitors of ACTRII Signaling

The ActRII signaling inhibitors described in this section and known in the art can be used in the methods provided herein. In certain embodiments, the ActRII signaling inhibitors described in this section can be used in the methods provided herein (See, Section 7.3).

Inhibitors of ActRII signaling receptors encompassed herein include ActRIIA signaling inhibitors and ActRIIB signaling inhibitors (see below). In certain embodiments, an ActRII signaling inhibitor is specific to ActRIIA signaling. In other embodiments, an ActRII signaling inhibitor is specific to ActRIIB signaling. In certain embodiments, an ActRII signaling inhibitor preferentially inhibits ActRIIA signaling. In other embodiments, an ActRII signaling inhibitor preferentially inhibits ActRIIB signaling. In certain embodiments, an ActRII signaling inhibitor inhibits both ActRIIA signaling and ActRIIB signaling.

In certain embodiments, inhibitors of ActRII signaling can be polypeptides comprising activin-binding domains of ActRII. Without being bound by theory, such activin-binding domain comprising polypeptides sequester activin and thereby prevent activin signaling. These activin-binding domain comprising polypeptides may comprise all or a portion of the extracellular domain of an ActRII (i.e., all or a portion of the extracellular domain of ActRIIA or all or a portion of the extracellular domain of ActRIIB) In specific embodiments, the extracellular domain of an ActRII is soluble.

In certain embodiments, the activin-binding domain comprising polypeptides are linked to an Fc portion of an antibody (i.e., a conjugate comprising an activin-binding domain comprising polypeptide of an ActRII receptor and an Fc portion of an antibody is generated). Without being bound by theory, the antibody portion confers increased stability on the conjugate. In certain embodiments, the activin-binding domain is linked to an Fc portion of an antibody via a linker, e.g., a peptide linker.

The inhibitors of ActRII signaling used in the compositions and methods described herein comprise molecules that inhibit ActRIIA signaling and/or ActRIIB signaling, directly or indirectly, either extracellularly or intracellularly. In some embodiments, the inhibitors of ActRIIA signaling and/or ActRIIB signaling used in the compositions and methods described herein inhibit ActRIIA signaling and/or ActRIIB signaling via interactions with the receptor(s) itself. In other embodiments, the inhibitors of ActRIIA signaling and/or ActRIIB signaling used in the compositions and methods described herein inhibit ActRIIA signaling and/or ActRIIB signaling via interactions with an ActRIIA and/or ActRIIB ligand, e.g., Activin.

### 7.6.1 Inhibitors of ACTRIIa Signaling

As used herein, the term “ActRIIA” refers to a family of activin receptor type IIA (ActRIIA) proteins from any species and variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain with a cysteine-rich region, a transmembrane domain, and a cytoplasmic domain with predicted serine/threonine kinase activity.

ActRIIA signaling inhibitors to be used in the compositions and methods described herein include, without limitation, activin-binding soluble ActRIIA polypeptides; antibodies that bind to activin (particularly the activin A or B subunits, also referred to as β_{A} or β_{B}) and disrupt ActRIIA binding; antibodies that bind to ActRIIA and disrupt activin binding; non-antibody proteins selected for activin or ActRIIA binding (see e.g., WO/2002/088171, WO/2006/055689, WO/2002/032925, WO/2005/037989, US 2003/0133939, and US 2005/0238646, each of which is incorporated herein by reference in its entirety, for examples of such proteins and methods for design and selection of same); and randomized peptides selected for activin or ActRIIA binding, which can be conjugated to an Fc domain.

In certain embodiments, two or more different proteins (or other moieties) with activin or ActRIIA binding activity, especially activin binders that block the type I (e.g., a soluble type I activin receptor) and type II (e.g., a soluble type II activin receptor) binding sites, respectively, may be linked together to create a bifunctional or multifunctional binding molecule that inhibits ActRIIA signaling and thus can be used in the compositions and methods described herein. In certain embodiments, Activin-ActRIIA signaling axis antagonists that inhibit ActRIIA signaling include nucleic acid aptamers, small molecules and other agents are used in the compositions and methods described herein include. 7.6.1.1 ActRIIA Signaling Inhibitors Comprising ActRIIA Polypeptides

The term “ActRIIA polypeptide” includes polypeptides comprising any naturally occurring polypeptide of an ActRIIA family member as well as any variants thereof (including mutants, fragments, fusions, and peptidomimetic forms) that retain a useful activity. For example, ActRIIA polypeptides include polypeptides derived from the sequence of any known ActRIIA having a sequence at least about 80% identical to the sequence of an ActRIIA polypeptide, and optionally at least 85%, 90%, 95%, 97%, 98%, 99% or greater identity. For example, an ActRIIA polypeptide may bind to and inhibit the function of an ActRIIA protein and/or activin. An ActRIIB polypeptide may be selected for its ability to promote bone growth and bone mineralization. Examples of ActRIIA polypeptides include human ActRIIA precursor polypeptide (SEQ ID NO: 1) and soluble human ActRIIA polypeptides (e.g., SEQ ID NOs: 2, 3, 7 and 12). With respect to the ActRIIA precursor polypeptide whose amino acid sequence is depicted at SEQ ID NO:1, the signal peptide of the human ActRIIA precursor polypeptide located at amino acid positions 1 to 20; the extracellular domain is located at amino acid positions 21 to 135 and the N-linked glycosylation sites of the human ActRIIA precursor polypeptide (SEQ ID NO: 1) are located at amino acid positions 43 and 56 of SEQ ID NO:1. The nucleic acid sequence encoding the human ActRIIB precursor polypeptide of SEQ ID NO:1 is disclosed as SEQ ID NO:4 (nucleotides 164-1705 of Genbank entry NM_001616). The nucleic acid sequence encoding the soluble human ActRIIA polypeptide of SEQ ID NO:2 is disclosed as SEQ ID NO:5. See Table 3 for a description of the sequences.

In specific embodiments, the ActRIIA polypeptides used in the compositions and methods described herein are soluble ActRIIA polypeptides. An extracellular domain of an ActRIIA protein can bind to activin and is generally soluble, and thus can be termed a soluble, activin-binding ActRIIA polypeptide. Thus, as used herein, the term “soluble ActRIIA polypeptide” generally refers to polypeptides comprising an extracellular domain of an ActRIIA protein, including any naturally occurring extracellular domain of an ActRIIA protein as well as any variants thereof (including mutants, fragments and peptidomimetic forms). Soluble ActRIIA polypeptides can bind to activin; however, the wild type ActRIIA protein does not exhibit significant selectivity in binding to activin versus GDF8/11. Native or altered ActRIIA proteins may be given added specificity for activin by coupling them with a second, activin-selective binding agent. Examples of soluble, activin-binding ActRIIA polypeptides include the soluble polypeptides illustrated in SEQ ID NOs: 2, 3, 7, 12 and 13. Other examples of soluble, activin-binding ActRIIA polypeptides comprise a signal sequence in addition to the extracellular domain of an ActRIIA protein, for example, the honey bee mellitin leader sequence (SEQ ID NO: 8), the tissue plasminogen activator (TPA) leader (SEQ ID NO: 9) or the native ActRIIA leader (SEQ ID NO: 10). The ActRIIA-hFc polypeptide illustrated in SEQ ID NO:13 uses a TPA leader.

In certain embodiments, the inhibitors of ActRIIA signaling used in the compositions and methods described herein comprise a conjugate/fusion protein comprising an activin-binding domain of ActRIIA linked to an Fc portion of an antibody. In certain embodiments, the activin-binding domain is linked to an Fc portion of an antibody via a linker, e.g., a peptide linker. Optionally, the Fc domain has one or more mutations at residues such as Asp-265, lysine 322, and Asn-434. In certain cases, the mutant Fc domain having one or more of these mutations (e.g., an Asp-265 mutation) has a reduced ability to bind to the Fcγ receptor relative to a wild-type Fc domain. In other cases, the mutant Fc domain having one or more of these mutations (e.g., an Asn-434 mutation) has an increased ability to bind to the MHC class I-related Fc-receptor (FcRN) relative to a wild-type Fc domain. Exemplary fusion proteins comprising a soluble extracellular domain of ActRIIA fused to an Fc domain are set forth in SEQ ID NOs: 6, 7, 12, and 13.

In a specific embodiment, the ActRIIA signaling inhibitors used in the compositions and methods described herein comprise the extracellular domain of ActRIIA, or a portion thereof, linked to an Fc portion of an antibody, wherein said ActRIIA signaling inhibitor comprises an amino acid sequence that is at least 75% identical to an amino acid sequence selected from SEQ ID NOs: 6, 7, 12, and 13. In another specific embodiment, the ActRIIA signaling inhibitors used in the compositions and methods described herein comprise the extracellular domain of ActRIIA, or a portion thereof, linked to an Fc portion of an antibody, wherein said ActRIIA signaling inhibitor comprises an amino acid sequence that is at least 80%, 85%, 90%, 95%, 96%, 97%, 98%, or 99% identical to an amino acid sequence selected from SEQ ID NOs: 6, 7, 12, and 13.

In certain embodiments, the inhibitors of ActRIIA signaling used in the compositions and methods described herein comprise a truncated form of an extracellular domain of ActRIIA. The truncation can be at the carboxy terminus and/or the amino terminus of the ActRIIA polypeptide. In certain embodiments, the truncation can be 1, 2, 3, 4, 5, 6, 7, 8, 9, 10, 11, 12, 13, 14, 15, 16, 17, 18, 19, 20, 21, 22, 23, 24, or 25 amino acids long relative to the mature ActRIIB polypeptide extracellular domain. In certain embodiments, the truncation can be 1, 2, 3, 4, 5, 6, 7, 8, 9, 10, 11, 12, 13, 14, 15, 16, 17, 18, 19, 20, 21, 22, 23, 24, or 25 N-terminal amino acids of the mature ActRIIA polypeptide extracellular domain. In certain embodiments, the truncation can be 1, 2, 3, 4, 5, 6, 7, 8, 9, 10, 11, 12, 13, 14, 15, 16, 17, 18, 19, 20, 21, 22, 23, 24, or 25 C-terminal amino acids of the mature ActRIIA polypeptide extracellular domain. For example, truncated forms of ActRIIA include polypeptides with amino acids 20-119; 20-128; 20-129; 20-130; 20-131; 20-132; 20-133; 20-134; 20-131; 21-131; 22-131; 23-131; 24-131; and 25-131, wherein the amino acid positions refer to the amino acid positions in SEQ ID NO:1.

In certain embodiments, the inhibitors of ActRIIA signaling used in the compositions and methods described herein comprise an extracellular domain of ActRIIA with one or more amino acid substitutions. In certain embodiments, the inhibitors of ActRIIA signaling used in the compositions and methods described herein comprise a truncated form of an ActRIIA extracellular domain that also carries an amino acid substitution.

In a specific embodiment, the ActRIIA signaling inhibitor to be used in the compositions and methods described herein is a fusion protein between the extracellular domain of the human ActRIIA receptor and the Fc portion of IgG1. In another specific embodiment, the ActRIIA signaling inhibitor to be used in the compositions and methods described herein is a fusion protein between a truncated extracellular domain of the human ActRIIA receptor and the Fc portion of IgG1. In another specific embodiment, the ActRIIA signaling inhibitor to be used in the compositions and methods described herein is a fusion protein between a truncated extracellular domain of the human ActRIIA receptor and the Fc portion of IgG1, wherein the truncated extracellular domain of the human ActRIIA receptor possesses one or more amino acid substitutions.

Functionally active fragments of ActRIIA polypeptides can be obtained, for example, by screening polypeptides recombinantly produced from the corresponding fragment of the nucleic acid encoding an ActRIIA polypeptide. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRIIA protein or signaling mediated by activin.

In addition, functionally active variants of ActRIIA polypeptides can be obtained, for example, by screening libraries of modified polypeptides recombinantly produced from the corresponding mutagenized nucleic acids encoding an ActRIIA polypeptide. The variants can be produced and tested to identify those that can function as antagonists (inhibitors) of ActRIIA protein or signaling mediated by activin. In certain embodiments, a functional variant of the ActRIIA polypeptides comprises an amino acid sequence that is at least 75% identical to an amino acid sequence selected from SEQ ID NOs: 2 or 3. In certain cases, the functional variant has an amino acid sequence at least 80%, 85%, 90%, 95%, 97%, 98%, 99% or 100% identical to an amino acid sequence selected from SEQ ID NOs: 2 or 3.

Functional variants may be generated, for example, by modifying the structure of an ActRIIA polypeptide for such purposes as enhancing therapeutic efficacy, or stability (e.g., ex vivo shelf life and resistance to proteolytic degradation in vivo). Such modified ActRIIA polypeptides when selected to retain activin binding, can be considered functional equivalents of the naturally-occurring ActRIIA polypeptides. Modified ActRIIA polypeptides can also be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of an ActRIIA polypeptide results in a functional homolog can be readily determined by assessing the ability of the variant ActRIIA polypeptide to produce a response in cells in a fashion similar to the wild-type ActRIIA polypeptide.

In certain embodiments, the ActRIIA signaling inhibitor to be used in the compositions and methods described herein provided herein may comprise an ActRIIA polypeptide having one or more specific mutations that can alter the glycosylation of the polypeptide. Such mutations may introduce or eliminate one or more glycosylation sites, such as O-linked or N-linked glycosylation sites. Asparagine-linked glycosylation recognition sites generally comprise a tripeptide sequence, asparagine-X-threonine (or asparagines-X-serine) (where “X” is any amino acid) which is specifically recognized by appropriate cellular glycosylation enzymes. The alteration may also be made by the addition of, or substitution by, one or more serine or threonine residues to the sequence of the wild-type ActRIIA polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on an ActRIIA polypeptide is by chemical or enzymatic coupling of glycosides to the ActRIIA polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on an ActRIIA polypeptide may be accomplished chemically and/or enzymatically. Chemical deglycosylation may involve, for example, exposure of the ActRIIA polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on ActRIIA polypeptides can be achieved by the use of a variety of endo- and exo-glycosidases as described by Thotakura et al. (1987) Meth. Enzymol. 138:350. The sequence of an ActRIIA polypeptide may be subsequent, as appropriate, depending on the type of expression system used, as mammalian, yeast, insect and plant cells may all introduce differing glycosylation patterns that can be affected by the amino acid sequence of the peptide. In general, ActRIIA proteins for use in humans can be expressed in a mammalian cell line that provides proper glycosylation, such as HEK293 or CHO cell lines, although other expression systems, such as other mammalian expression cell lines, yeast cell lines with engineered glycosylation enzymes and insect cells, are expected to be useful as well.

Further provided herein are methods of generating mutants, particularly sets of combinatorial mutants of an ActRIIA polypeptide, as well as truncation mutants; pools of combinatorial mutants are especially useful for identifying functional variant sequences. The purpose of screening such combinatorial libraries may be to generate, for example, ActRIIA polypeptide variants which can act as either agonists or antagonist, or alternatively, which possess novel activities all together. A variety of screening assays are provided below, and such assays may be used to evaluate variants. For example, an ActRIIA polypeptide variant may be screened for ability to bind to an ActRIIA ligand, to prevent binding of an ActRIIA ligand to an ActRIIA polypeptide or to interfere with signaling caused by an ActRIIA ligand.

Combinatorially-derived variants can be generated which have a selective or generally increased potency relative to a naturally occurring ActRIIA polypeptide. Likewise, mutagenesis can give rise to variants which have intracellular half-lives dramatically different than the corresponding a wild-type ActRIIA polypeptide. For example, the altered protein can be rendered either more stable or less stable to proteolytic degradation or other cellular processes which result in destruction of, or otherwise inactivation of a native ActRIIA polypeptide. Such variants, and the genes which encode them, can be utilized to alter ActRIIA polypeptide levels by modulating the half-life of the ActRIIA polypeptides. For instance, a short half-life can give rise to more transient biological effects and can allow tighter control of recombinant ActRIIA polypeptide levels within the subject. In an Fc fusion protein, mutations may be made in the linker (if any) and/or the Fc portion to alter the half-life of the protein.

A combinatorial library may be produced by way of a degenerate library of genes encoding a library of polypeptides which each include at least a portion of potential ActRIIA polypeptide sequences. For instance, a mixture of synthetic oligonucleotides can be enzymatically ligated into gene sequences such that the degenerate set of potential ActRIIA polypeptide nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate vector for expression. The synthesis of degenerate oligonucleotides is well known in the art (see, for example, Narang, S A (1983) Tetrahedron 39:3; Itakura et al., (1981) Recombinant DNA, Proc. 3rd Cleveland Sympos. Macromolecules, ed. AG Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, ActRIIA polypeptide variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of ActRIIA polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of ActRIIA polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include activin binding assays and activin-mediated cell signaling assays.

In certain embodiments, ActRIIA polypeptides used in the inhibitors of the methods and compositions described herein may further comprise post-translational modifications in addition to any that are naturally present in the ActRIIA polypeptides. Such modifications may include, but are not limited to, acetylation, carboxylation, glycosylation, phosphorylation, lipidation, and acylation. As a result, the modified ActRIIA polypeptides may contain non-amino acid elements, such as polyethylene glycols, lipids, poly- or mono-saccharide, and phosphates. Effects of such non-amino acid elements on the functionality of an ActRIIA polypeptide may be tested by any method known to the skilled artisan. When an ActRIIA polypeptide is produced in cells by cleaving a nascent form of the ActRIIA polypeptide, post-translational processing may also be important for correct folding and/or function of the protein. Different cells (such as CHO, HeLa, MDCK, 293, W138, NIH-3T3 or HEK293) have specific cellular machinery and characteristic mechanisms for such post-translational activities and may be chosen to ensure the correct modification and processing of the ActRIIA polypeptides.

In certain aspects, functional variants or modified forms of the ActRIIA polypeptides used in the inhibitors of the methods and compositions described herein include fusion proteins having at least a portion of the ActRIIA polypeptides and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ActRIIA polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus hemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins there from. The liberated proteins can then be isolated from the fusion domain by subsequent chromatographic separation. In certain preferred embodiments, an ActRIIA polypeptide is fused with a domain that stabilizes the ActRIIA polypeptide in vivo (a “stabilizer” domain). By “stabilizing” is meant anything that increases serum half life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of fusion domains that may be selected include multimerizing (e.g., dimerizing, tetramerizing) domains and functional domains (that confer an additional biological function, such as further stimulation of bone growth or muscle growth, as desired).

It is understood that different elements of the fusion proteins may be arranged in any manner that is consistent with the desired functionality. For example, an ActRIIA polypeptide may be placed C-terminal to a heterologous domain, or, alternatively, a heterologous domain may be placed C-terminal to an ActRIIA polypeptide. The ActRIIA polypeptide domain and the heterologous domain need not be adjacent in a fusion protein, and additional domains or amino acid sequences may be included C- or N-terminal to either domain or between the domains.

In certain embodiments, the ActRIIA polypeptides used in the inhibitors of the methods and compositions described herein may contain one or more modifications that are capable of stabilizing the ActRIIA polypeptides. For example, such modifications may enhance the in vitro half life of the ActRIIA polypeptides, enhance circulatory half life of the ActRIIA polypeptides or reduce proteolytic degradation of the ActRIIA polypeptides. Such stabilizing modifications may include, but are not limited to, fusion proteins (including, for example, fusion proteins comprising an ActRIIA polypeptide and a stabilizer domain), modifications of a glycosylation site (including, for example, addition of a glycosylation site to an ActRIIA polypeptide), and modifications of carbohydrate moiety (including, for example, removal of carbohydrate moieties from an ActRIIA polypeptide). In the case of fusion proteins, an ActRIIA polypeptide is fused to a stabilizer domain such as an IgG molecule (e.g., an Fc domain). As used herein, the term “stabilizer domain” not only refers to a fusion domain (e.g., Fc) as in the case of fusion proteins, but also includes nonproteinaceous modifications such as a carbohydrate moiety, or nonproteinaceous polymer, such as polyethylene glycol.

In certain embodiments, isolated and/or purified forms of ActRIIA polypeptides, which are isolated from, or otherwise substantially free of, other proteins can be used with the methods and compositions described herein. ActRIIA polypeptides can generally be produced by expression from recombinant nucleic acids.

In certain aspects, the ActRIIA polypeptides used in the compositions and methods described herein are generated using isolated and/or recombinant nucleic acids encoding any of the ActRIIA polypeptides (e.g., soluble ActRIIA polypeptides), including fragments, functional variants and fusion proteins disclosed herein. For example, SEQ ID NO: 4 encodes the naturally occurring human ActRIIA precursor polypeptide, while SEQ ID NO: 5 encodes the processed extracellular domain of ActRIIA. Such nucleic acids may be single-stranded or double stranded. Such nucleic acids may be DNA or RNA molecules. These nucleic acids may be used, for example, in methods for making ActRIIA polypeptides or as direct therapeutic agents (e.g., in a gene therapy approach).

In certain aspects, nucleic acids encoding ActRIIA polypeptides may include nucleic acids that are variants of SEQ ID NO: 4 or 5. Variant nucleotide sequences include sequences that differ by one or more nucleotide substitutions, additions or deletions, such as allelic variants.

In certain embodiments, isolated or recombinant nucleic acid sequences encoding ActRIIA polypeptides may be least 80%, 85%, 90%, 95%, 97%, 98%, 99% or 100% identical to SEQ ID NO: 4 or 5. One of ordinary skill in the art will appreciate that nucleic acid sequences complementary to SEQ ID NO: 4 or 5, and variants of SEQ ID NO: 4 or 5 may be used in the production of ActRIIA polypeptides suitable for use in the methods and compositions described herein. In further embodiments, such nucleic acid sequences can be isolated, recombinant, and/or fused to a heterologous nucleotide sequence, or be from a DNA library.

In other embodiments, nucleic acids used in the production of ActRIIA polypeptides suitable for use in the methods and compositions described herein may include nucleotide sequences that hybridize under highly stringent conditions to the nucleotide sequence designated in SEQ ID NO: 4 or 5, complement sequence of SEQ ID NO: 4 or 5, or fragments thereof. One of ordinary skill in the art will understand that appropriate stringency conditions which promote DNA hybridization can be varied. For example, one can perform the hybridization at 6.0 times sodium chloride/sodium citrate (SSC) at about 45 degree Celsius, followed by a wash of 2.0 times SSC at 50 degree Celsius. For example, the salt concentration in the wash step can be selected from a low stringency of about 2.0 times SSC at 50 degree Celsius to a high stringency of about 0.2 times SSC at 50 degree Celsius. In addition, the temperature in the wash step can be increased from low stringency conditions at room temperature, about 22 degree Celsius, to high stringency conditions at about 65 degree Celsius. Both temperature and salt may be varied, or temperature or salt concentration may be held constant while the other variable is changed. In one embodiment, nucleic acids which hybridize under low stringency conditions of 6 times SSC at room temperature followed by a wash at 2 times SSC at room temperature can be used with the methods and compositions described herein.

Isolated nucleic acids which differ from the nucleic acids as set forth in SEQ ID NOs: 4 or 5 due to degeneracy in the genetic code also can be used in the production of ActRIIA polypeptides suitable for use in the methods and compositions described herein. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation.

In certain embodiments, the recombinant nucleic acids may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated herein. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects, the a nucleic acid used in the production of ActRIIA polypeptides suitable for use in the methods and compositions described herein can be provided in an expression vector comprising a nucleotide sequence encoding an ActRIIA polypeptide and operably linked to at least one regulatory sequence. Regulatory sequences are art-recognized and are selected to direct expression of the ActRIIA polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding an ActRIIA polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast .alpha.-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid used in the production of ActRIIA polypeptides suitable for use in the methods and compositions described herein can be produced by ligating the cloned gene, or a portion thereof, into a vector suitable for expression in either prokaryotic cells, eukaryotic cells (yeast, avian, insect or mammalian), or both. Expression vehicles for production of a recombinant ActRIIA polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the .beta.-gal containing pBlueBac III).

Vectors can be designed for production of the subject ActRIIA polypeptides in CHO cells, such as a Pcmv-Script vector (Stratagene, La Jolla, Calif), pcDNA4 vectors (Invitrogen, Carlsbad, Calif.) and pCI-neo vectors (Promega, Madison, Wis.). As will be apparent, the subject gene constructs can be used to cause expression of the subject ActRIIA polypeptides in cells propagated in culture, e.g., to produce proteins, including fusion proteins or variant proteins, for purification.

Host cells transfected with a recombinant gene including a coding sequence (e.g., SEQ ID NO: 4 or 5) for one or more of the subject ActRIIA polypeptides can be used in the production of ActRIIA polypeptides suitable for use in the methods and compositions described herein. The host cell may be any prokaryotic or eukaryotic cell. For example, an ActRIIA polypeptide provided herein may be expressed in bacterial cells such as E. coli, insect cells (e.g., using a baculovirus expression system), yeast, or mammalian cells. Other suitable host cells are known to those skilled in the art.

Accordingly, provided herein are methods of producing the ActRIIA polypeptides. For example, a host cell transfected with an expression vector encoding an ActRIIA polypeptide can be cultured under appropriate conditions to allow expression of the ActRIIA polypeptide to occur. The ActRIIA polypeptide may be secreted and isolated from a mixture of cells and medium containing the ActRIIA polypeptide. Alternatively, the ActRIIA polypeptide may be retained cytoplasmically or in a membrane fraction and the cells harvested, lysed and the protein isolated. A cell culture includes host cells, media and other byproducts. Suitable media for cell culture are well known in the art. The subject ActRIIA polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ActRIIA polypeptides and affinity purification with an agent that binds to a domain fused to the ActRIIA polypeptide (e.g., a protein A column may be used to purify an ActRIIA-Fc fusion). In a preferred embodiment, the ActRIIA polypeptide is a fusion protein containing a domain which facilitates its purification. In one embodiment, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. As demonstrated herein, ActRIIA-hFc protein was purified to a purity of >98% as determined by size exclusion chromatography and >95% as determined by SDS PAGE. This level of purity was sufficient to achieve desirable effects on bone in mice and an acceptable safety profile in mice, rats and non-human primates.

In another embodiment, a fusion gene coding for a purification leader sequence, such as a poly-(His)/enterokinase cleavage site sequence at the N-terminus of the desired portion of a recombinant ActRIIA polypeptide, can allow purification of the expressed fusion protein by affinity chromatography using a Ni²⁺ metal resin. The purification leader sequence can then be subsequently removed by treatment with enterokinase to provide the purified ActRIIA polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

ActRIIA-Fc fusion protein can be expressed in stably transfected CHO-DUKX Bl 1 cells from a pAID4 vector (SV40 ori/enhancer, CMV promoter), using a tissue plasminogen leader sequence of SEQ ID NO:9. The Fc portion is a human IgGl Fc sequence, as shown in SEQ ID NO:7. In certain embodiments, upon expression, the protein contained has, on average, between about 1.5 and 2.5 moles of sialic acid per molecule of ActRIIA-Fc fusion protein.

In certain embodiments, the long serum half-life of an ActRIIA-Fc fusion can be 25-32 days in human subjects. Additionally, the CHO cell expressed material can have a higher affinity for activin B ligand than that reported for an ActRIIA-hFc fusion protein expressed in human 293 cells (del Re et al., J Biol Chem. 2004 Dec. 17; 279(51):53126-35). Additionally, without being bound by theory, the use of the TPA leader sequence provided greater production than other leader sequences and, unlike ActRIIA-Fc expressed with a native leader, may provide a highly pure N-terminal sequence. Use of the native leader sequence may result in two major species of ActRIIA-Fc, each having a different N-terminal sequence.

### 7.6.2 Inhibitors of ACTRIIb Signaling

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms of the receptor. Members of the ActRIIB family are generally transmembrane proteins, composed of a ligand-binding extracellular domain with a cysteine-rich region, a transmembrane domain, and a cytoplasmic domain with predicted serine/threonine kinase activity.

ActRIIB signaling inhibitors to be used in the compositions and methods described herein include, without limitation, activin-binding soluble ActRIIB polypeptides; antibodies that bind to activin (particularly the activin A or B subunits, also referred to as β_{A} or β_{B}) and disrupt ActRIIB binding; antibodies that bind to ActRIIB and disrupt activin binding; non-antibody proteins selected for activin or ActRIIB binding; and randomized peptides selected for activin or ActRIIB binding, which can be conjugated to an Fc domain.

In certain embodiments, two or more different proteins (or other moieties) with activin or ActRIIB binding activity, especially activin binders that block the type I (e.g., a soluble type I activin receptor) and type II (e.g., a soluble type II activin receptor) binding sites, respectively, may be linked together to create a bifunctional or multifunctional binding molecule that inhibits ActRIIB and thus can be used in the compositions and methods described herein include. In certain embodiments, Activin-ActRIIB signaling axis antagonists that inhibit ActRIIB include nucleic acid aptamers, small molecules and other agents are used in the compositions and methods described herein include.

### 7.6.2.1 ActRIIB Signaling Inhibitors Comprising ActRIIB Polypeptides

As used herein, the term “ActRIIB polypeptide” refers to polypeptides comprising any naturally occurring polypeptide of an ActRIIB family member as well as any variants thereof (including mutants, fragments, fusions, and peptidomimetic forms) that retain a useful activity. For example, ActRIIB polypeptides include polypeptides derived from the sequence of any known ActRIIB receptor having a sequence at least about 80% identical to the sequence of an ActRIIB polypeptide, and optionally at least 85%, 90%, 95%, 96%, 97%, 98%, 99% or greater identity. For example, an ActRIIB polypeptide may bind to and inhibit the function of an ActRIIB protein and/or activin. An example of an ActRIIB polypeptide includes the human ActRIIB precursor polypeptide (SEQ ID NO:16 or SEQ ID NO:28). With respect to the ActRIIB precursor polypeptide whose amino acid sequence is depicted as SEQ ID NO:16 or SEQ ID NO:28 (i.e., the human ActRIIB precursor polypeptide), the signal peptide of the ActRIIB precursor polypeptide is located at amino acids 1 to 18; the extracellular domain is located at amino acids 19 to 134 and the potential N-linked glycosylation sites are located at amino acid positions 42 and 65. The nucleic acid sequence encoding the human ActRIIB precursor polypeptide of SEQ ID NO:16 is disclosed as SEQ ID NO:19 (SEQ ID NO:19 provides an alanine at the codon corresponding to amino acid position 64, but could be readily modified by one of skill in the art using methods known in the art to provide an arginine at the codon corresponding to amino acid position 64 instead). See Table 3 for a description of the sequences.

The numbering of amino acids for all of the ActRIIB-related polypeptides described herein is based on the amino acid numbering for SEQ ID NO:16 and SEQ ID NO:28 (which only differ in the amino acid expressed at position 64), unless specifically designated otherwise. For example, if an ActRIIB polypeptide is described as having a substitution/mutation at amino acid position 79, then it is to be understood that position 79 refers to the 79^{th} amino acid in SEQ ID NO:16 or SEQ ID NO:28, from which the ActRIIB polypeptide is derived. Likewise, if an ActRIIB polypeptide is described as having an alanine or an arginine at amino acid position 64, then it is to be understood that position 64 refers to the 64^{th} amino acid in SEQ ID NO:16 or SEQ ID NO:28, from which the ActRIIB polypeptide is derived.

In certain embodiments, the inhibitors of ActRIIB signaling used in the compositions and methods described herein comprise polypeptides comprising an activin-binding domain of ActRIIB In some embodiments, the activin-binding domains of ActRIIB comprise the extracellular domain of ActRIIB, or a portion thereof. In specific embodiments, the extracellular domain or portion thereof of ActRIIB is soluble. Illustrative modified forms of ActRIIB polypeptides are disclosed in U.S. Patent Application Publication Nos. 20090005308 and 20100068215, the disclosures of which are incorporated herein by reference in their entireties. Illustrative modified forms of ActRIIB polypeptides are also disclosed in International Patent Application Publication Nos. WO 2008/097541 and WO 2010/019261, the disclosures of which are incorporated herein by reference in their entireties.

In specific embodiments, the ActRIIB polypeptides used in the compositions and methods described herein are soluble ActRIIB polypeptides. The term “soluble ActRIIB polypeptide” generally refers to polypeptides comprising an extracellular domain of an ActRIIB protein, including any naturally occurring extracellular domain of an ActRIIB protein as well as any variants thereof (including mutants, fragments and peptidomimetic forms). Soluble ActRIIB polypeptides can bind to activin; however, the wild type ActRIIB protein does not exhibit significant selectivity in binding to activin versus GDF8/11. In certain embodiments, altered forms of ActRIIB with different binding properties can be used in the methods provided herein. Such altered forms are disclosed, e.g., in international patent application publication Nos. WO 2006/012627 and WO 2010/019261, the disclosures of which are incorporated herein by reference in their entireties. Native or altered ActRIIB proteins may be given added specificity for activin by coupling them with a second, activin-selective binding agent. Exemplary soluble ActRIIB polypeptides include the extracellular domain of a human ActRIIB polypeptide (e.g., SEQ ID NOs: 17, 18, 23, 26, 27, 29, 30, 31, 32, 33, 36, 37, 42, and 43).

An Fc fusion protein having the ActRIIB extracellular sequence disclosed by Hilden et al. (Blood, 1994, 83(8):2163-70), which has an alanine at the position corresponding to amino acid 64 of the ActRIIB precursor amino acid sequence, i.e., SEQ ID NO: 16 (herein referred to as “A64”), has been demonstrated to possess a relatively low affinity for activin and GDF-11. By contrast, an Fc fusion protein with an arginine at position 64 of the ActRIIB precursor amino acid sequence (herein referred to as “R64”) has an affinity for activin and GDF-11 in the low nanomolar to high picomolar range (see, e.g., U.S. Patent Application Publication No. 20100068215, the disclosure of which is herein incorporated in its entirety). See, also, International Publication No. WO 2010/019261, the disclosure of which is herein incorporated in its entirety. An ActRIIB precursor amino acid sequence with an arginine at position 64 is presented in SEQ ID NO:28. As such, in certain embodiments, the ActRIIB polypeptides used in accordance with the compositions and methods described herein may comprise either (i) an alanine at the position corresponding to amino acid 64 of the ActRIIB precursor amino acid sequence, i.e., SEQ ID NO: 16; or (ii) an arginine at position 64 of the ActRIIB precursor amino acid sequence, i.e., SEQ ID NO: 28. In other embodiments, the ActRIIB polypeptides used in accordance with the compositions and methods described herein may comprise an amino acid that is not alanine or arginine at the position corresponding to amino acid 64 of the ActRIIB precursor amino acid sequence, i.e., SEQ ID NO: 16 or SEQ ID NO:28.

It has been shown that a deletion of the proline knot at the C-terminus of the extracellular domain of ActRIIB reduces the affinity of the receptor for activin (see, e.g., Attisano et al., Cell, 1992, 68(1):97-108). An ActRIIB-Fc fusion protein containing amino acids 20-119 of SEQ ID NO: 28 (i.e., SEQ ID NO:32), “ActRIM(20-119)-Fc” has reduced binding to GDF-11 and activin relative to an ActRIIB-Fc fusion protein containing amino acids 20-134 of SEQ ID NO: 28 (i.e., SEQ ID NO:31), “ActRIM(20-134)-Fc”, which includes the proline knot region and the complete juxtamembrane domain. However, an ActRIIB-Fc fusion protein containing amino acids 20-129 of SEQ ID NO: 28, “ActRIM(20-129)-Fc” retains similar but somewhat reduced activity relative to the non-truncated extracellular domain of ActRIIB, even though the proline knot region is disrupted. Thus, ActRIIB polypeptides comprising extracellular domains that stop at amino acid 134, 133, 132, 131, 130 and 129 of SEQ ID NO: 28 (or SEQ ID NO:16) are all expected to be active, but constructs stopping at amino acid 134 or 133 may be most active. Similarly, mutations at any of residues 129-134 are not expected to alter ligand binding affinity by large margins, as indicated by the fact that mutations of P129 and P130 of SEQ ID NO: 28 do not substantially decrease ligand binding. Therefore, the ActRIIB polypeptides used in accordance with the methods and compositions described herein may end as early as amino acid 109 (i.e., the final cysteine) of SEQ ID NO:28 (or SEQ ID NO:16), however, forms ending at or between amino acid positions 109 and 119 of SEQ ID NO:28 (or SEQ ID NO:16) are expected to have reduced ligand binding ability.

Amino acid 29 of SEQ ID NO:16 and SEQ ID NO:28 represents the initial cysteine in the ActRIIB precursor sequence. It is expected that an ActRIIB polypeptide beginning at amino acid 29 of the N-terminus of SEQ ID NO:16 or SEQ ID NO:28, or before these amino acid positions, will retain ligand binding activity. An alanine to asparagine mutation at position 24 of SEQ ID NO:16 or SEQ ID NO:28 introduces an N-linked glycosylation sequence without substantially affecting ligand binding. This confirms that mutations in the region between the signal cleavage peptide and the cysteine cross-linked region, corresponding to amino acids 20-29 of SEQ ID NO:16 or SEQ ID NO:28, are well tolerated. In particular, ActRIIB polypeptides beginning at amino acid position 20, 21, 22, 23 and 24 of SEQ ID NO:16 or SEQ ID NO:28 will retain activity, and ActRIIB polypeptides beginning at amino acid positions 25, 26, 27, 28 and 29 of SEQ ID NO:16 or SEQ ID NO:28 are also expected to retain activity. An ActRIIB polypeptide beginning at amino acid position 22, 23, 24 or 25 of SEQ ID NO:16 or SEQ ID NO:28 will have the most activity.

Taken together, the active portions (i.e., ActRIIB polypeptides) of the ActRIIB precursor protein (i.e., SEQ ID NO:16 or SEQ ID NO:28) to be used in accordance with the methods and compositions described herein will generally comprise amino acids 29-109 of SEQ ID NO:16 or SEQ ID NO:28, and such ActRIIB polypeptides may, for example, begin at a residue corresponding to any one of amino acids 19-29 of SEQ ID NO:16 or SEQ ID NO:28 and end at a position corresponding to any one of amino acids 109-134 of SEQ ID NO:16 or SEQ ID NO:28. Specific examples of ActRIIB polypeptides encompassed herein include those that begin at an amino acid position from 19-29, 20-29 or 21-29 of SEQ ID NO:16 or SEQ ID NO:28 and end at an amino acid position from 119-134, 119-133 or 129-134, 129-133 of SEQ ID NO:16 or SEQ ID NO:28. Other specific examples of ActRIIB polypeptides encompassed herein include those that begin at an amino acid position from 20-24 (or 21-24, or 22-25) of SEQ ID NO:16 or SEQ ID NO:28 and end at an amino acid position from 109-134 (or 109-133), 119-134 (or 119-133) or 129-134 (or 129-133) of SEQ ID NO:16 or SEQ ID NO:28. Variant ActRIIB polypeptides falling within these ranges are also contemplated, particularly those having at least 80%, 85%, 90%, 91%, 92%, 93%, 94%, 95%, 96%, 97%, 98%, or 99% sequence identity or sequence homology to the corresponding portion of SEQ ID NO:16 or SEQ ID NO:28.

In certain embodiments, the inhibitors of ActRIIB signaling used in the compositions and methods described herein comprise a truncated form of an extracellular domain of ActRIIB The truncation can be at the carboxy terminus and/or the amino terminus of the ActRIIB polypeptide. In certain embodiments, the truncation can be 1, 2, 3, 4, 5, 6, 7, 8, 9, 10, 11, 12, 13, 14, 15, 16, 17, 18, 19, 20, 21, 22, 23, 24, or 25 amino acids long relative to the mature ActRIIB polypeptide extracellular domain. In certain embodiments, the truncation can be 1, 2, 3, 4, 5, 6, 7, 8, 9, 10, 11, 12, 13, 14, 15, 16, 17, 18, 19, 20, 21, 22, 23, 24, or 25 N-terminal amino acids of the mature ActRIIB polypeptide extracellular domain. In certain embodiments, the truncation can be 1, 2, 3, 4, 5, 6, 7, 8, 9, 10, 11, 12, 13, 14, 15, 16, 17, 18, 19, 20, 21, 22, 23, 24, or 25 C-terminal amino acids of the mature ActRIIB polypeptide extracellular domain. For example, truncated forms of ActRIIB include polypeptides with amino acids 20-119; 20-128; 20-129; 20-130; 20-131; 20-132; 20-133; 20-134; 20-131; 21-131; 22-131; 23-131; 24-131; and 25-131, wherein the amino acid positions refer to the amino acid positions in SEQ ID NO:16 or SEQ ID NO:28.

Additional exemplary truncated forms of ActRIIB include (i) polypeptides beginning at amino acids at any of amino acids 21-29 of SEQ ID NO:16 or SEQ ID NO:28 (optionally beginning at 22-25 of SEQ ID NO:16 or SEQ ID NO:28) and ending at any of amino acids 109-134 of SEQ ID NO:16 or SEQ ID NO:28; (ii) polypeptides beginning at any of amino acids 20-29 of SEQ ID NO:16 or SEQ ID NO:28 (optionally beginning at 22-25 of SEQ ID NO:16 or SEQ ID NO:28) and ending at any of amino acids 109-133 of SEQ ID NO:16 or SEQ ID NO:28; (iii) polypeptides beginning at any of amino acids 20-24 of SEQ ID NO:16 or SEQ ID NO:28 (optionally beginning at 22-25 of SEQ ID NO:16 or SEQ ID NO:28) and ending at any of amino acids 109-133 of SEQ ID NO:16 or SEQ ID NO:28; (iv) polypeptides beginning at any of amino acids 21-24 of SEQ ID NO:16 or SEQ ID NO:28 and ending at any of amino acids 109-134 of SEQ ID NO:16 or SEQ ID NO:28; (v) polypeptides beginning at any of amino acids 20-24 of SEQ ID NO:16 or SEQ ID NO:28 and ending at any of amino acids 118-133 of SEQ ID NO:16 or SEQ ID NO:28; (vi) polypeptides beginning at any of amino acids 21-24 of SEQ ID NO:16 or SEQ ID NO:28 and ending at any of amino acids 118-134 of SEQ ID NO:16 or SEQ ID NO:28; (vii) polypeptides beginning at any of amino acids 20-24 of SEQ ID NO:16 or SEQ ID NO:28 and ending at any of amino acids 128-133 of SEQ ID NO:16 or SEQ ID NO:28; (viii) polypeptides beginning at any of amino acids 20-24 of SEQ ID NO:16 or SEQ ID NO:28 and ending at any of amino acids 128-133 of SEQ ID NO:16 or SEQ ID NO:28; (ix) polypeptides beginning at any of amino acids 21-29 of SEQ ID NO:16 or SEQ ID NO:28 and ending at any of amino acids 118-134 of SEQ ID NO:16 or SEQ ID NO:28; (x) polypeptides beginning at any of amino acids 20-29 of SEQ ID NO:16 or SEQ ID NO:28 and ending at any of amino acids 118-133 of SEQ ID NO:16 or SEQ ID NO:28; (xi) polypeptides beginning at any of amino acids 21-29 of SEQ ID NO:16 or SEQ ID NO:28 and ending at any of amino acids 128-134 of SEQ ID NO:16 or SEQ ID NO:28; and (xii) polypeptides beginning at any of amino acids 20-29 of SEQ ID NO:16 or SEQ ID NO:28 and ending at any of amino acids 128-133 of SEQ ID NO:16 or SEQ ID NO:28. In a specific embodiment, an ActRIIB polypeptides comprises, consists essentially of, or consists of, an amino acid sequence beginning at amino acid position 25 of SEQ ID NO:16 or SEQ ID NO:28 and ending at amino acid position 131 of SEQ ID NO:16 or SEQ ID NO:28. In another specific embodiment, an ActRIIB polypeptide consists of, or consists essentially of, the amino acid sequence of SEQ ID NO:17, 18, 23, 26, 27, 29, 30, 31, 32, 33, 36, 37, 42, or 43.

Any of the ActRIIB polypeptides used in the compositions and methods described herein may be produced as a homodimer. Any of the ActRIIB polypeptides used in the compositions and methods described herein may be formulated as a fusion protein having a heterologous portion that comprises a constant region from an IgG heavy chain, such as an Fc domain. Any of the ActRIIB polypeptides used in the compositions and methods described herein may comprise an acidic amino acid at the position corresponding to position 79 of SEQ ID NO:16 or SEQ ID NO:28, optionally in combination with one or more additional amino acid substitutions, deletions or insertions relative to SEQ ID NO:16 or SEQ ID NO:28.

In specific embodiments, the inhibitors of ActRIIB signaling used in the compositions and methods described herein comprise an extracellular domain of ActRIIB with one or more amino acid substitutions/mutations. Such an amino acid substitution/mutation can be, for example, an exchange from the leucine at amino acid position 79 of SEQ ID NO:16 or SEQ ID NO:28 to an acidic amino acid, such as aspartic acid or glutamic acid. For example, position L79 of SEQ ID NO:16 or SEQ ID NO:28 may be altered in ActRIIB extracellular domain polypeptides to confer altered activin-myostatin (GDF-11) binding properties. L79A and L79P mutations reduce GDF-11 binding to a greater extent than activin binding. L79E and L79D mutations retain GDF-11 binding, while demonstrating greatly reduced activin binding.

In certain embodiments, the inhibitors of ActRIIB signaling used in the compositions and methods described herein comprise a truncated form of an ActRIIB extracellular domain that also carries an amino acid substitution, e.g., an exchange from the leucine at amino acid position 79 of SEQ ID NO:16 or SEQ ID NO:28 to an acidic amino acid, such as aspartic acid or glutamic acid. In a specific embodiment, the truncated form of an extracellular domain of ActRIIB polypeptide that also carries an amino acid substitution used in the compositions and methods described herein is SEQ ID NO:23. Forms of ActRIIB that are truncated and/or carry one or more amino acid substitutions can be linked to an Fc domain of an antibody as discussed above.

Functionally active fragments of ActRIIB polypeptides can be obtained, for example, by screening polypeptides recombinantly produced from the corresponding fragment of the nucleic acid encoding an ActRIIB polypeptide. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRIIB protein or signaling mediated by activin.

In addition, functionally active variants of ActRIIB polypeptides can be obtained, for example, by screening libraries of modified polypeptides recombinantly produced from the corresponding mutagenized nucleic acids encoding an ActRIIB polypeptide. The variants can be produced and tested to identify those that can function as antagonists (inhibitors) of ActRIIB protein or signaling mediated by activin. In certain embodiments, a functional variant of the ActRIIB polypeptides comprises an amino acid sequence that is at least 75% identical to an amino acid sequence selected from SEQ ID NO:17, 18, 23, 26, 27, 29, 30, 31, 32, 33, 36, 37, 42, and 43. In certain embodiments, the functional variant has an amino acid sequence at least 80%, 85%, 90%, 95%, 96%, 97%, 98%, or 99% identical to an amino acid sequence selected from SEQ ID NO:17, 18, 23, 26, 27, 29, 30, 31, 32, 33, 36, 37, 42, and 43.

Functional variants may be generated, for example, by modifying the structure of an ActRIIB polypeptide for such purposes as enhancing therapeutic efficacy, or stability (e.g., ex vivo shelf life and resistance to proteolytic degradation in vivo). Such modified ActRIIB polypeptides when selected to retain activin binding, are considered functional equivalents of the naturally-occurring ActRIIB polypeptides. Modified ActRIIB polypeptides can also be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of an ActRIIB polypeptide results in a functional homolog can be readily determined by assessing the ability of the variant ActRIIB polypeptide to produce a response in cells in a fashion similar to the wild-type ActRIIB polypeptide.

ActRIIB polypeptide mutants, particularly sets of combinatorial mutants of an ActRIIB polypeptide, as well as truncation mutants; pools of combinatorial mutants are especially useful for identifying functional variant sequences can be used in the methods and compositions described herein. The purpose of screening such combinatorial libraries may be to generate, for example, ActRIIB polypeptide variants which can act as either agonists or antagonist, or alternatively, which possess novel activities all together.

It has been demonstrated that the ligand binding pocket of ActRIIB is defined by residues Y31, N33, N35, L38 through T41, E47, E50, Q53 through K55, L57, H58, Y60, S62, K74, W78 through N83, Y85, R87, A92, and E94 through F101 of SEQ ID NO:16 or SEQ ID NO:28. At these positions, it is expected that conservative mutations will be tolerated, although a K74A mutation is well-tolerated, as are R40A, K55A, F82A and mutations at position L79. R40 is a K in Xenopus, indicating that basic amino acids at this position will be tolerated. Q53 is R in bovine ActRIIB and K in Xenopus ActRIIB, and therefore amino acids including R, K, Q, N and H will be tolerated at this position. Thus, a general formula for an ActRIIB polypeptide for use in the methods and compositions described herein is one that comprises amino acids 29-109 of SEQ ID NO:16 or SEQ ID NO:28, but optionally beginning at an amino acid position ranging from 20-24 or 22-25 of SEQ ID NO:16 or SEQ ID NO:28 and ending at an amino acid position ranging from 129-134 of SEQ ID NO:16 or SEQ ID NO:28, and comprising no more than 1, 2, 5, or 15 conservative amino acid changes in the ligand binding pocket, and zero, one or more non-conservative alterations at amino acid positions 40, 53, 55, 74, 79 and/or 82 of SEQ ID NO:16 or SEQ ID NO:28 in the ligand binding pocket. Such an ActRIIB polypeptide may retain greater than 80%, 90%, 95% or 99% sequence identity or sequence homology to the sequence of amino acids 29-109 of SEQ ID NO:16 or SEQ ID NO:28. Sites outside the binding pocket, at which variability may be particularly well tolerated, include the amino and carboxy termini of the extracellular domain of ActRIIB, and positions 42-46 and 65-73. An asparagine to alanine alteration at position 65 of SEQ ID NO:16 or SEQ ID NO:28 (N65A) actually improves ligand binding in the A64 background, and is thus expected to have no detrimental effect on ligand binding in the R64 background. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64.

As a specific example of an ActRIIB polypeptide with a mutation in the ligand binding domain, the positively-charged amino acid residue Asp (D80) of the ligand-binding domain of ActRIIB can be mutated to a different amino acid residue such that the variant ActRIIB polypeptide preferentially binds to GDF8, but not activin. In a specific embodiment, the D80 residue is changed to an amino acid residue selected from the group consisting of: an uncharged amino acid residue, a negative amino acid residue, and a hydrophobic amino acid residue. As a further specific example, the hydrophobic residue L79 can be altered to the acidic amino acids aspartic acid or glutamic acid to greatly reduce activin binding while retaining GDF11 binding. As will be recognized by one of skill in the art, most of the described mutations, variants or modifications may be made at the nucleic acid level or, in some cases, by post translational modification or chemical synthesis. Such techniques are well known in the art.

In specific embodiments, the inhibitors of ActRIIB signaling used in the compositions and methods described herein comprise a conjugate/fusion protein comprising an extracellular domain (e.g., an activin-binding domain) of an ActRIIB receptor linked to an Fc portion of an antibody. Such conjugate/fusion proteins may comprise any of the ActRIIB polypeptides disclosed herein (e.g., any of SEQ ID NOs:17, 18, 23, 26, 27, 29, 30, 31, 32, 33, 36, 37, 42, or 43), any ActRIIB polypeptides known in the art, or any ActRIIB polypeptides generated using methods known in the art and/or provided herein.

In certain embodiments, the extracellular domain is linked to an Fc portion of an antibody via a linker, e.g., a peptide linker. Exemplary linkers include short polypeptide sequences such as 2-10, 2-5, 2-4, 2-3 amino acid residues (e.g., glycine residues), such as, for example, a Gly-Gly-Gly linker. In a specific embodiment, the linker comprises the amino acid sequence Gly-Gly-Gly (GGG). In another specific embodiment, the linker comprises the amino acid sequence Thr-Gly-Gly-Gly (TGGG). Optionally, the Fc domain has one or more mutations at residues such as Asp-265, lysine 322, and Asn-434. In certain cases, the mutant Fc domain having one or more of these mutations (e.g., an Asp-265 mutation) has a reduced ability to bind to the Fcγ receptor relative to a wild-type Fc domain. In other cases, the mutant Fc domain having one or more of these mutations (e.g., an Asn-434 mutation) has an increased ability to bind to the MHC class I-related Fc-receptor (FcRN) relative to a wild-type Fc domain. Exemplary fusion proteins comprising a soluble extracellular domain of ActRIIB fused to an Fc domain are set forth in SEQ ID NOs:20, 21, 24, 25, 34, 35, 38, 39, 40, 41, 44, 46, and 47.

In a specific embodiment, the ActRIIB signaling inhibitors used in the compositions and methods described herein comprise the extracellular domain of ActRIIB, or a portion thereof, linked to an Fc portion of an antibody, wherein said ActRIIB signaling inhibitor comprises an amino acid sequence that is at least 75% identical to an amino acid sequence selected from SEQ ID NOs:20, 21, 24, 25, 34, 35, 38, 39, 40, 41, 44, 46, and 47. In another specific embodiment, the ActRIIB signaling inhibitors used in the compositions and methods described herein comprise the extracellular domain of ActRIIB, or a portion thereof, linked to an Fc portion of an antibody, wherein said ActRIIB signaling inhibitor comprises an amino acid sequence that is at least 80%, 85%, 90%, 95%, 96%, 97%, 98%, or 99% identical to an amino acid sequence selected from SEQ ID NOs:20, 21, 24, 25, 34, 35, 38, 39, 40, 41, 44, 46, and 47.

In a specific embodiment, the ActRIIB signaling inhibitor to be used in the compositions and methods described herein is a fusion protein between the extracellular domain of the human ActRIIB receptor and the Fc portion of IgG1. In another specific embodiment, the ActRIIB signaling inhibitor to be used in the compositions and methods described herein is a fusion protein between a truncated extracellular domain of the human ActRIIB receptor and the Fc portion of IgG1. In another specific embodiment, the ActRIIB signaling inhibitor to be used in the compositions and methods described herein is a fusion protein between a truncated extracellular domain of the human ActRIIB receptor and the Fc portion of IgG1, wherein the truncated extracellular domain of the human ActRIIB receptor possesses an amino acid substitution at the amino acid position corresponding to amino acid 79 of SEQ ID NO:16 or SEQ ID NO:28. In one embodiment, the amino acid substitution at the amino acid position corresponding to amino acid 79 of SEQ ID NO:16 or SEQ ID NO:28 is substitution of Leucine for Aspartic Acid (i.e., an L79D mutation).

In a specific embodiment, the ActRIIB signaling inhibitor to be used in the compositions and methods described herein is SEQ ID NO:24 or 25, which represents a fusion protein between the extracellular domain of the human ActRIIB receptor and the Fc portion of IgG1, wherein said ActRIIB extracellular domain comprises amino acids 25-131 of SEQ ID NO:28 with an L79D mutation. The nucleic acid sequence encoding the ActRIIB-Fc fusion protein of SEQ ID NO:24 is presented in SEQ ID NO:45.

In another specific embodiment, the ActRIIB signaling inhibitor to be used in the compositions and methods described herein is SEQ ID NO:34 or 35, which represents a fusion protein between the extracellular domain of the human ActRIIB receptor and the Fc portion of IgG1, wherein said ActRIIB extracellular domain comprises amino acids 25-131 of SEQ ID NO:16 with an L79D mutation.

Asparagine-linked glycosylation recognition sites generally comprise a tripeptide sequence, asparagine-X-threonine (or asparagine-X-serine) (where “X” is any amino acid) which is specifically recognized by appropriate cellular glycosylation enzymes. The alteration may also be made by the addition of, or substitution by, one or more serine or threonine residues to the sequence of the wild-type ActRIIB polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on an ActRIIB polypeptide is by chemical or enzymatic coupling of glycosides to the ActRIIB polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in International Patent Application No. WO 87/05330 published Sep. 11, 1987, and in Aplin and Wriston (1981) CRC Crit. Rev. Biochem., pp. 259-306, incorporated by reference herein. Removal of one or more carbohydrate moieties present on an ActRIIB polypeptide may be accomplished chemically and/or enzymatically. Chemical deglycosylation may involve, for example, exposure of the ActRIIB polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on ActRIIB polypeptides can be achieved by the use of a variety of endo- and exo-glycosidases as described by Thotakura et al. (1987) Meth. Enzymol. 138:350. The sequence of an ActRIIB polypeptide may be subsequent, as appropriate, depending on the type of expression system used, as mammalian, yeast, insect and plant cells may all introduce differing glycosylation patterns that can be affected by the amino acid sequence of the peptide. In general, ActRIIB proteins for use in humans will be expressed in a mammalian cell line that provides proper glycosylation, such as HEK293 or CHO cell lines, although other expression systems, such as other mammalian expression cell lines, yeast cell lines with engineered glycosylation enzymes and insect cells, are expected to be useful as well.

In specific embodiments, mutated ActRIIB polypeptides comprising the addition of a further N-linked glycosylation site (N-X-S/T) that increases the serum half-life of an ActRIIB-Fc fusion protein, relative to the ActRIIB(R64)-Fc form can be used in the methods and compositions described herein. In a specific embodiment, introduction of an asparagine at position 24 of SEQ ID NO:16 or SEQ ID NO:28 (A24N) results in the creation of an NXT sequence that confers a longer half-life. Other NX(T/S) sequences can be found at 42-44 (NQS) and 65-67 (NSS), although the latter may not be efficiently glycosylated with the R at position 64 (i.e., in R64 polypeptides). N-X-S/T sequences may be generally introduced at positions outside the ligand binding pocket of ActRIIB, which is detailed above. Particularly suitable sites for the introduction of non-endogenous N-X-S/T sequences include amino acids 20-29, 20-24, 22-25, 109-134, 120-134 or 129-134 of SEQ ID NO:16 or SEQ ID NO:28. N-X-S/T sequences may also be introduced into the linker between the ActRIIB sequence and the Fc or other fusion component. Such a site may be introduced with minimal effort by introducing an N in the correct position with respect to a pre-existing S or T, or by introducing an S or T at a position corresponding to a pre-existing N. Thus, desirable alterations that would create an N-linked glycosylation site are: A24N, R64N, S67N (possibly combined with an N65A alteration), E106N, R112N, G120N, E123N, P129N, A132N, R112S and R112T (with all amino acid positions corresponding to the positions they can be found in SEQ ID NO:16 or SEQ ID NO:28). Any S that is predicted to be glycosylated may be altered to a T without creating an immunogenic site, because of the protection afforded by the glycosylation. Likewise, any T that is predicted to be glycosylated may be altered to an S. Thus the alterations S67T and S44T are encompassed herein. Likewise, in an A24N variant, an S26T alteration may be used. Accordingly, an ActRIIB polypeptide may include one or more additional, non-endogenous N-linked glycosylation consensus sequences.

A variety of screening assays may be used to evaluate ActRIIB polypeptide variants. For example, an ActRIIB polypeptide variant may be screened for ability to bind to an ActRIIB ligand, to prevent binding of an ActRIIB ligand to an ActRIIB polypeptide or to interfere with signaling caused by an ActRIIB ligand. The activity of an ActRIIB polypeptide or its variants may also be tested in a cell-based or in vivo assay.

Combinatorially-derived variants can be generated which have a selective or generally increased potency relative to a naturally occurring ActRIIB polypeptide. Likewise, mutagenesis can give rise to variants which have intracellular half-lives dramatically different than the corresponding wild-type ActRIIB polypeptide. For example, the altered protein can be rendered either more stable or less stable to proteolytic degradation or other cellular processes which result in destruction of, or otherwise inactivation of a native ActRIIB polypeptide. Such variants, and the genes which encode them, can be utilized to alter ActRIIB polypeptide levels by modulating the half-life of the ActRIIB polypeptides. For instance, a short half-life can give rise to more transient biological effects and can allow tighter control of recombinant ActRIIB polypeptide levels within the subject. In an Fc fusion protein, mutations may be made in the linker (if any) and/or the Fc portion to alter the half-life of the protein.

A combinatorial library may be produced by way of a degenerate library of genes encoding a library of polypeptides which each include at least a portion of potential ActRIIB polypeptide sequences. For instance, a mixture of synthetic oligonucleotides can be enzymatically ligated into gene sequences such that the degenerate set of potential ActRIIB polypeptide nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate vector for expression. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. AG Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, ActRIIB polypeptide variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of ActRIIB polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of ActRIIB polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include activin binding assays and activin-mediated cell signaling assays.

In certain embodiments, ActRIIB polypeptides used in the methods and compositions described herein may further comprise post-translational modifications in addition to any that are naturally present in the ActRIIB polypeptides. Such modifications include, but are not limited to, acetylation, carboxylation, glycosylation, phosphorylation, lipidation, and acylation. As a result, the modified ActRIIB polypeptides may contain non-amino acid elements, such as polyethylene glycols, lipids, poly- or mono-saccharide, and phosphates. Effects of such non-amino acid elements on the functionality of an ActRIIB polypeptide may be tested by any method known to the skilled artisan. When an ActRIIB polypeptide is produced in cells by cleaving a nascent form of the ActRIIB polypeptide, post-translational processing may also be important for correct folding and/or function of the protein. Different cells (such as CHO, HeLa, MDCK, 293, W138, NIH-3T3 or HEK293) have specific cellular machinery and characteristic mechanisms for such post-translational activities and may be chosen to ensure the correct modification and processing of the ActRIIB polypeptides.

In certain aspects, functional variants or modified forms of the ActRIIB polypeptides include fusion proteins having at least a portion of the ActRIIB polypeptides and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS6) fusion partners. As another example, a fusion domain may be selected so as to facilitate detection of the ActRIIB polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus hemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins there from. The liberated proteins can then be isolated from the fusion domain by subsequent chromatographic separation. In certain preferred embodiments, an ActRIIB polypeptide is fused with a domain that stabilizes the ActRIIB polypeptide in vivo (a “stabilizer” domain). By “stabilizing” is meant anything that increases serum half life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of fusion domains that may be selected include multimerizing (e.g., dimerizing, tetramerizing) domains and functional domains (that confer an additional biological function, such as further stimulation of bone growth or muscle growth, as desired).

It is understood that different elements of the fusion proteins may be arranged in any manner that is consistent with the desired functionality. For example, an ActRIIB polypeptide may be placed C-terminal to a heterologous domain, or, alternatively, a heterologous domain may be placed C-terminal to an ActRIIB polypeptide. The ActRIIB polypeptide domain and the heterologous domain need not be adjacent in a fusion protein, and additional domains or amino acid sequences may be included C- or N-terminal to either domain or between the domains.

In certain embodiments, the ActRIIB polypeptides used in the methods and compositions described herein contain one or more modifications that are capable of stabilizing the ActRIIB polypeptides. For example, such modifications enhance the in vitro half life of the ActRIIB polypeptides, enhance circulatory half life of the ActRIIB polypeptides or reduce proteolytic degradation of the ActRIIB polypeptides. Such stabilizing modifications include, but are not limited to, fusion proteins (including, for example, fusion proteins comprising an ActRIIB polypeptide and a stabilizer domain), modifications of a glycosylation site (including, for example, addition of a glycosylation site to an ActRIIB polypeptide), and modifications of carbohydrate moiety (including, for example, removal of carbohydrate moieties from an ActRIIB polypeptide). In the case of fusion proteins, an ActRIIB polypeptide is fused to a stabilizer domain such as an IgG molecule (e.g., an Fc domain). As used herein, the term “stabilizer domain” not only refers to a fusion domain (e.g., Fc) as in the case of fusion proteins, but also includes nonproteinaceous modifications such as a carbohydrate moiety, or nonproteinaceous polymer, such as polyethylene glycol.

In certain embodiments, the methods and compositions described herein use isolated or purified ActRIIB polypeptides, i.e., ActRIIB polypeptides which are isolated from, or otherwise substantially free of, other proteins can be used with the methods and compositions described herein. ActRIIB polypeptides will generally be produced by expression from recombinant nucleic acids.

In certain aspects, the ActRIIB polypeptides used in the methods and compositions described herein are encoded by isolated and/or recombinant nucleic acids, including fragments, functional variants and fusion proteins disclosed herein. For example, SEQ ID NO:19 encodes the naturally occurring human ActRIIB precursor polypeptide. The subject nucleic acids may be single-stranded or double stranded. Such nucleic acids may be DNA or RNA molecules. These nucleic acids may be used, for example, in methods for making ActRIIB polypeptides or as direct therapeutic agents (e.g., in a gene therapy approach).

In certain aspects, the nucleic acids that can be used to produce ActRIIB polypeptides suitable for use in the methods and compositions described herein are further understood to include nucleic acids that are variants of SEQ ID NO: 19 as well as variants of those nucleic acid sequences that encode soluble ActRIIB polypeptides (e.g., nucleic acids that encode SEQ ID NOs: 17, 18, 23, 26, 27, 29, 30, 31, 32, 33, 36, 37, 42, and 43). Variant nucleotide sequences include sequences that differ by one or more nucleotide substitutions, additions or deletions, such as allelic variants.

In certain embodiments, the isolated or recombinant nucleic acid sequences that can be used to produce ActRIIB polypeptides suitable for use in the methods and compositions described herein are at least 80%, 85%, 90%, 95%, 97%, 98%, 99% or 100% identical to SEQ ID NO:19 or those nucleic acid sequences that encode soluble ActRIIB polypeptides (e.g., nucleic acids that encode SEQ ID NOs: 17, 18, 23, 26, 27, 29, 30, 31, 32, 33, 36, 37, 42, and 43). One of ordinary skill in the art will appreciate that nucleic acid sequences complementary to SEQ ID NO:19 or those nucleic acid sequences that encode soluble ActRIIB polypeptides (e.g., nucleic acids that encode SEQ ID NOs: 17, 18, 23, 26, 27, 29, 30, 31, 32, 33, 36, 37, 42, and 43), and variants of SEQ ID NO:19 or those nucleic acid sequences that encode soluble ActRIIB polypeptides (e.g., nucleic acids that encode SEQ ID NOs: 17, 18, 23, 26, 27, 29, 30, 31, 32, 33, 36, 37, 42, and 43) can be used with the methods and compositions described herein. In further embodiments, the nucleic acid sequences can be isolated, recombinant, and/or fused with a heterologous nucleotide sequence, or in a DNA library.

In other embodiments, nucleic acids that can be used to produce ActRIIB polypeptides suitable for use in the methods and compositions described herein include nucleotide sequences that hybridize under highly stringent conditions to the nucleotide sequence designated in SEQ ID NO:19 or those nucleic acid sequences that encode soluble ActRIIB polypeptides (e.g., nucleic acids that encode SEQ ID NOs: 17, 18, 23, 26, 27, 29, 30, 31, 32, 33, 36, 37, 42, and 43), complement sequence of SEQ ID NO:19 or those nucleic acid sequences that encode soluble ActRIIB polypeptides (e.g., nucleic acids that encode SEQ ID NOs: 17, 18, 23, 26, 27, 29, 30, 31, 32, 33, 36, 37, 42, and 43), or fragments thereof. One of ordinary skill in the art will understand that appropriate stringency conditions which promote DNA hybridization can be varied. For example, one can perform the hybridization at 6.0 times sodium chloride/sodium citrate (SSC) at about 45 degree Celsius, followed by a wash of 2.0 times SSC at 50 degree Celsius. For example, the salt concentration in the wash step can be selected from a low stringency of about 2.0 times SSC at 50 degree Celsius to a high stringency of about 0.2 times SSC at 50 degree Celsius. In addition, the temperature in the wash step can be increased from low stringency conditions at room temperature, about 22 degree Celsius, to high stringency conditions at about 65 degree Celsius. Both temperature and salt may be varied, or temperature or salt concentration may be held constant while the other variable is changed. In one embodiment, nucleic acids which hybridize under low stringency conditions of 6 times SSC at room temperature followed by a wash at 2 times SSC at room temperature can be used with the methods and compositions described herein.

Isolated nucleic acids which differ from the nucleic acids as set forth in SEQ ID NO:19 or those nucleic acid sequences that encode soluble ActRIIB polypeptides (e.g., nucleic acids that encode SEQ ID NOs: 17, 18, 23, 26, 27, 29, 30, 31, 32, 33, 36, 37, 42, and 43) due to degeneracy in the genetic code can also be used to produce ActRIIB polypeptides suitable for use in the methods and compositions described herein. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms can be used with the methods and compositions described herein.

In certain embodiments, the recombinant nucleic acids that can be used to produce ActRIIB polypeptides suitable for use in the methods and compositions described herein may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art can be used with the methods and compositions described herein. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects, the nucleic acids that can be used to produce ActRIIB polypeptides suitable for use in the methods and compositions described herein are provided in an expression vector comprising a nucleotide sequence encoding an ActRIIB polypeptide and operably linked to at least one regulatory sequence. Regulatory sequences are art-recognized and are selected to direct expression of the ActRIIB polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding an ActRIIB polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast .alpha.-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid can be produced by ligating the cloned gene, or a portion thereof, into a vector suitable for expression in either prokaryotic cells, eukaryotic cells (yeast, avian, insect or mammalian), or both. Expression vehicles for production of a recombinant ActRIIB polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the.beta.-gal containing pBlueBac III).

In one embodiment, a vector can be designed for production of the ActRIIB polypeptides used in the methods and compositions described herein in CHO cells, such as a Pcmv-Script vector (Stratagene, La Jolla, Calif), pcDNA4 vectors (Invitrogen, Carlsbad, Calif.) and pCI-neo vectors (Promega, Madison, Wis.). As will be apparent, the subject gene constructs can be used to cause expression of the subject ActRIIB polypeptides in cells propagated in culture, e.g., to produce proteins, including fusion proteins or variant proteins, for purification.

Host cells transfected with a recombinant gene including a coding sequence (e.g., SEQ ID NO:19 or those nucleic acid sequences that encode soluble ActRIIB polypeptides (e.g., nucleic acids that encode SEQ ID NOs: 17, 18, 23, 26, 27, 29, 30, 31, 32, 33, 36, 37, 42, and 43)) for one or more of the subject ActRIIB polypeptides can be used to produce ActRIIB polypeptides suitable for use in the methods and compositions described herein. The host cell may be any prokaryotic or eukaryotic cell. For example, an ActRIIB polypeptide may be expressed in bacterial cells such as E. coli, insect cells (e.g., using a baculovirus expression system), yeast, or mammalian cells. Other suitable host cells are known to those skilled in the art.

Accordingly, provided herein are methods of producing the ActRIIB polypeptides used in the methods and compositions described herein. For example, a host cell transfected with an expression vector encoding an ActRIIB polypeptide can be cultured under appropriate conditions to allow expression of the ActRIIB polypeptide to occur. The ActRIIB polypeptide may be secreted and isolated from a mixture of cells and medium containing the ActRIIB polypeptide. Alternatively, the ActRIIB polypeptide may be retained cytoplasmically or in a membrane fraction and the cells harvested, lysed and the protein isolated. A cell culture includes host cells, media and other byproducts. Suitable media for cell culture are well known in the art. The subject ActRIIB polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ActRIIB polypeptides and affinity purification with an agent that binds to a domain fused to the ActRIIB polypeptide (e.g., a protein A column may be used to purify an ActRIIB-Fc fusion). In a preferred embodiment, the ActRIIB polypeptide is a fusion protein containing a domain which facilitates its purification. In a preferred embodiment, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. As demonstrated herein, ActRIIB-hFc protein was purified to a purity of >98% as determined by size exclusion chromatography and >95% as determined by SDS PAGE. This level of purity was sufficient to achieve desirable effects on bone in mice and an acceptable safety profile in mice, rats and non-human primates.

In another embodiment, a fusion gene coding for a purification leader sequence, such as a poly-(His)/enterokinase cleavage site sequence at the N-terminus of the desired portion of the recombinant ActRIIB polypeptide, can allow purification of the expressed fusion protein by affinity chromatography using a Ni2+ metal resin. The purification leader sequence can then be subsequently removed by treatment with enterokinase to provide the purified ActRIIB polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

ActRIIB-Fc fusion protein can be expressed in stably transfected CHO-DUKX Bl 1 cells from a pAID4 vector (SV40 ori/enhancer, CMV promoter), using a tissue plasminogen leader sequence of SEQ ID NO:8. The Fc portion can comprise a human IgGl Fc sequence, as shown in SEQ ID NO:7. In certain embodiments, upon expression, the protein contained has, on average, between about 1.5 and 2.5 moles of sialic acid per molecule of ActRIIB-Fc fusion protein.

In certain embodiments, the long serum half-life of an ActRIIB-Fc fusion can be 25-32 days in human subjects. Additionally, the CHO cell expressed material can have a higher affinity for activin B ligand than that reported for an ActRIIB-hFc fusion protein expressed in human 293 cells (del Re et al., J Biol Chem. 2004 Dec. 17; 279(51):53126-35). Additionally, without being bound by theory, the use of the TPA leader sequence provided greater production than other leader sequences and, unlike ActRIIB-Fc expressed with a native leader, may provide a highly pure N-terminal sequence. Use of the native leader sequence may result in two major species of ActRIIB-Fc, each having a different N-terminal sequence.

### 7.6.3 Other ACTRII Receptor Signaling Inhibitors

In certain embodiments, the inhibitors of ActRII signaling used in the compositions and methods described herein are nucleic acid compounds.

Examples of categories of nucleic acid compounds that inhibit ActRII receptors include antisense nucleic acids, siRNA or RNAi constructs and catalytic nucleic acid constructs. A nucleic acid compound may be single- or double-stranded. A double-stranded compound may also include regions of overhang or non-complementarity, where one or the other of the strands is single-stranded. A single-stranded compound may include regions of self-complementarity, meaning that the compound may form a so-called “hairpin” or “stem-loop” structure, with a region of double helical structure.

In certain embodiments, the nucleic acid compounds that inhibit ActRII receptors may comprise a nucleotide sequence that is complementary to a region consisting of no more than 1000, no more than 500, no more than 250, no more than 100 or no more than 50, 35, 30, 25, 22, 20 or 18 nucleotides of the full-length ActRII receptor nucleic acid sequence or activin nucleic acid sequence (e.g., the nucleic acid sequence of an activin A or activin B subunit, also referred to as β_{A} or β_{B}). In specific embodiments, the region of complementarity will be at least 8 nucleotides, and optionally at least 10 or at least 15 nucleotides, and optionally between 15 and 25 nucleotides. A region of complementarity may fall within an intron, a coding sequence or a noncoding sequence of the target transcript, such as the coding sequence portion. Generally, a nucleic acid compound that inhibits an ActRII receptor will have a length of about 8 to about 500 nucleotides or base pairs in length, and optionally the length will be about 14 to about 50 nucleotides. A nucleic acid compound that inhibits an ActRII receptor may be a DNA (particularly for use as an antisense), an RNA, or an RNA:DNA hybrid. Any one strand may include a mixture of DNA and RNA, as well as modified forms that cannot readily be classified as either DNA or RNA. Likewise, a double stranded nucleic acid compound may be DNA:DNA, DNA:RNA, or RNA:RNA, and any one strand may also include a mixture of DNA and RNA, as well as modified forms that cannot readily be classified as either DNA or RNA.

The nucleic acid compounds that inhibit an ActRII receptor may include any of a variety of modifications, including one or modifications to the backbone (the sugar-phosphate portion in a natural nucleic acid, including internucleotide linkages) or the base portion (the purine or pyrimidine portion of a natural nucleic acid). In certain embodiments, an antisense nucleic acid compound will have a length of about 15 to about 30 nucleotides and will often contain one or more modifications to improve certain characteristics, such as stability in the serum, stability in a cell, or stability in a place where the compound is likely to be delivered, such as, e.g., the stomach in the case of orally delivered compounds and the lung for inhaled compounds. In the case of an RNAi construct, the strand complementary to the target transcript will generally be RNA or modifications thereof. The other strand may be RNA, DNA, or any other variation. The duplex portion of double stranded or single stranded “hairpin” RNAi construct may, in certain embodiments, have a length of 18 to 40 nucleotides in length and optionally about 21 to 23 nucleotides in length, so long as it serves as a Dicer substrate. Catalytic or enzymatic nucleic acids may be ribozymes or DNA enzymes and may also contain modified forms. In certain embodiments, nucleic acid compounds that inhibit ActRII receptors may inhibit expression of their target by about 50%, 60%, 70%, 75%, 80%, 85%, 90%, 95%, 99%, or more under physiological conditions and at a concentration where a nonsense or sense control has little or no effect. Concentrations for testing the effect of nucleic acid compounds include 1, 5, 10 micromolar, or more.

In other embodiments, the inhibitors of ActRII signaling used in the compositions and methods described herein are antibodies. Such antibodies include antibodies that bind to activin (particularly the activin A or B subunits, also referred to as BA or BB) and disrupt ActRII receptor binding; and antibodies that bind to ActRII receptor polypeptides (e.g., a soluble ActRIIA or soluble ActRIIB polypeptide) and disrupt activin binding.

By using immunogens derived from an ActRII receptor polypeptide or an activin polypeptide, anti-protein/anti-peptide antisera or monoclonal antibodies can be made by standard protocols (see, for example, Antibodies: A Laboratory Manual ed. by Harlow and Lane (Cold Spring Harbor Press: 1988)). A mammal, such as a mouse, a hamster or rabbit can be immunized with an immunogenic form of the ActRII receptor polypeptide, an antigenic fragment which is capable of eliciting an antibody response, or a fusion protein. Techniques for conferring immunogenicity on a protein or peptide include conjugation to carriers or other techniques well known in the art. An immunogenic portion of an ActRII receptor or activin polypeptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of an ActRII receptor polypeptide, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with an ActRII receptor polypeptide and monoclonal antibodies isolated from a culture comprising such hybridoma cells.

The term “antibody” as used herein is intended to include fragments thereof which are also specifically reactive with a subject polypeptide. Antibodies can be fragmented using conventional techniques and the fragments screened for utility in the same manner as described above for whole antibodies. For example, F(ab)2 fragments can be generated by treating antibody with pepsin. The resulting F(ab)2 fragment can be treated to reduce disulfide bridges to produce Fab fragments. An antibody is further intended to include bispecific, single-chain, chimeric, humanized and fully human molecules having affinity for an ActRII receptor or activin polypeptide conferred by at least one CDR region of the antibody. An antibody may further comprise a label attached thereto and able to be detected (e.g., the label can be a radioisotope, fluorescent compound, enzyme or enzyme co-factor).

In certain embodiments, the antibody is a recombinant antibody, which term encompasses any antibody generated in part by techniques of molecular biology, including CDR-grafted or chimeric antibodies, human or other antibodies assembled from library-selected antibody domains, single chain antibodies and single domain antibodies (e.g., human V_{H} proteins or camelid V_{HH} proteins). In certain embodiments, an antibody can be a monoclonal antibody, and in certain embodiments. For example, a method for generating a monoclonal antibody that binds specifically to an ActRII receptor polypeptide or activin polypeptide may comprise administering to a mouse an amount of an immunogenic composition comprising the antigen polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monoclonal antibody that binds specifically to the antigen. Once obtained, a hybridoma can be propagated in a cell culture, optionally in culture conditions where the hybridoma-derived cells produce the monoclonal antibody that binds specifically to the antigen. The monoclonal antibody may be purified from the cell culture.

The adjective “specifically reactive with” as used in reference to an antibody is intended to mean, as is generally understood in the art, that the antibody is sufficiently selective between the antigen of interest (e.g., an ActRII receptor polypeptide) and other antigens that are not of interest that the antibody is useful for, at minimum, detecting the presence of the antigen of interest in a particular type of biological sample. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less. Given the extraordinarily tight binding between activin and an ActRII receptor, it is expected that a neutralizing anti-activin or anti-ActRII receptor antibody would generally have a dissociation constant of 10⁻¹⁰ or less.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interaction between antibodies and antigens to identify particularly desirable antibodies. Such techniques include ELISAs, surface plasmon resonance binding assays (e.g., the Biacore™ binding assay, Biacore AB, Uppsala, Sweden), sandwich assays (e.g., the paramagnetic bead system of IGEN International, Inc., Gaithersburg, Md.), Western blots, immunoprecipitation assays, and immunohistochemistry.

In certain embodiments, ActRII signaling inhibitors to be used in the compositions and methods described herein include alternative forms of activin, particularly those with alterations in the type I receptor binding domain can bind to type II receptors and fail to form an active ternary complex. In certain embodiments, nucleic acids, such as antisense molecules, siRNAs or ribozymes that inhibit activin A, B, C or E, or, particularly, ActRII receptor expression, can be used in the compositions and methods described herein. In certain embodiments, the ActRII signaling inhibitors to be used in the compositions and methods described herein exhibit selectivity for inhibiting GDF11-mediated signaling versus other members of the TGF-beta family, particularly with respect to GDF8 and activin.

In other embodiments, the inhibitors of ActRII signaling used in the compositions and methods described herein are non-antibody proteins with ActRII receptor antagonist activity, including inhibin (i.e., inhibin alpha subunit), follistatin (e.g., follistatin-288 and follistatin-315), Cerberus, follistatin related protein (“FSRP”), endoglin, activin C, alpha(2)-macroglobulin, and an M108A (methionine to alanine change at position 108) mutant activin A.

In a specific embodiment, the ActRII signaling inhibitor to be used in the compositions and methods described herein is a follistatin polypeptide that antagonizes activin bioactivity and/or binds to activin. The term “follistatin polypeptide” includes polypeptides comprising any naturally occurring polypeptide of follistatin as well as any variants thereof (including mutants, fragments, fusions, and peptidomimetic forms) that retain a useful activity, and further includes any functional monomer or multimer of follistatin. Variants of follistatin polypeptides that retain activin binding properties can be identified based on previous studies involving follistatin and activin interactions. For example, WO2008/030367, which is included by reference herein in its entirety, discloses specific follistatin domains (“FSDs”) that are shown to be important for activin binding. Follistatin polypeptides include polypeptides derived from the sequence of any known follistatin having a sequence at least about 80% identical to the sequence of a follistatin polypeptide, and optionally at least 85%, 90%, 95%, 96%, 97%, 98%, 99% or greater identity. Examples of follistatin polypeptides include the mature follistatin polypeptide or shorter isoforms or other variants of the human follistatin precursor polypeptide as described, for example, in WO2005/025601, which is included by reference herein in its entirety.

In a specific embodiment, the ActRII signaling inhibitor to be used in the compositions and methods described herein is a follistatin-like related gene (FLRG) that antagonizes activin bioactivity and/or binds to activin. The term “FLRG polypeptide” includes polypeptides comprising any naturally occurring polypeptide of FLRG as well as any variants thereof (including mutants, fragments, fusions, and peptidomimetic forms) that retain a useful activity. Variants of FLRG polypeptides that retain activin binding properties can be identified using routine methods to assay FLRG and activin interactions. See, for example, U.S. Pat. No. 6,537,966, which is included by reference herein in its entirety. FLRG polypeptides include polypeptides derived from the sequence of any known FLRG having a sequence at least about 80% identical to the sequence of an FLRG polypeptide, and optionally at least 85%, 90%, 95%, 96%, 97%, 98%, 99% or greater identity.

In certain embodiments, functional variants or modified forms of the follistatin polypeptides and FLRG polypeptides include fusion proteins having at least a portion of the follistatin polypeptides or FLRG polypeptides and one or more fusion domains, such as, for example, domains that facilitate isolation, detection, stabilization or multimerization of the polypeptide. Suitable fusion domains are discussed in detail above with reference to the ActRIIA and ActRIIB polypeptides. In one embodiment, an ActRII signaling inhibitor is a fusion protein comprising an activin binding portion of a follistatin polypeptide fused to an Fc domain. In another embodiment, an ActRII signaling inhibitor is a fusion protein comprising an activin binding portion of an FLRG polypeptide fused to an Fc domain.

### 7.7 Assays

Various ActRII polypeptide variants, or soluble ActRII polypeptide variants, may be tested for their ability to inhibit ActRII. In addition, compounds can be tested for their ability to inhibit ActRII. Once inhibitors of ActRII signaling activity are confirmed, these compounds can be used with the methods provided herein. ActRII can be ActRIIA or ActRIIB The assays below are described for ActRIIA but can be performed analogously for ActRIIB

### 7.7.1 Reference Population

In certain embodiments, the size of the reference population can be 1, 5, 10, 25, 50, 75, 100, 200, 250, 300, 400, 500, or 1000 individuals. In certain embodiments, the reference population consists of random volunteers. In certain embodiments, the reference population consists of healthy people. In certain embodiments, the reference population consists of people of the same age, weight, and/or gender as the patient population as described in Section 7.5. In certain embodiments, the reference population consists of people without beta-thalassemia.

7.7.2 Assessing Protein Levels and/or Activities

The level of a protein, such as hemoglobin, fetal hemoglobin, or GDF11, can be determined by any method known in the art or described herein. For example, the level of the protein, such as hemoglobin, fetal hemoglobin, or GDF11 in a tissue sample can be determined by assessing (e.g., quantifying) transcribed RNA of the protein in the sample using, e.g., Northern blotting, PCR analysis, real time PCR analysis, or any other technique known in the art or described herein. In one embodiment, the level of the protein in a tissue sample can be determined by assessing (e.g., quantifying) mRNA of the protein in the sample.

The level of a protein, such as hemoglobin, fetal hemoglobin, or GDF11, in a tissue sample can also be determined by assessing (e.g., quantifying) the level of protein expression of the protein in the sample using, e.g., immunohistochemical analysis, Western blotting, ELISA, immunoprecipitation, flow cytometry analysis, or any other technique known in the art or described herein. In particular embodiments, the level of the protein is determined by a method capable of quantifying the amount of the protein present in a tissue sample of a patient (e.g., in human serum), and/or capable of detecting the correction of the level of protein following treatment with an activin type II receptor signaling inhibitor. In one embodiment, the level of the protein in a tissue sample is determined by assessing (e.g., quantifying) protein expression of the protein in the sample using ELISA.

### 7.7.3 Reduced Serum Ferritin Levels

Serum ferritin levels can be determined according to assay(s) known to one skilled in the art. Typically, adult males have a serum ferritin concentration of between 24 and 336 ng/mL. Typically, adult females of between 11 and 307 ng/mL.

### 7.7.4 Iron Levels

Iron levels, such as, e.g., liver or myocardial iron levels can be determined according to assay(s) known to one skilled in the art. For example, iron levels (e.g., liver iron concentration or myocardial iron concentration) can be determined by magnetic resonance imaging.

### 7.7.5 Red Blood Cell Morphology

Red blood cell morphology can be evaluated according to assay(s) known to one skilled in the art such as, for example, blood smears. The ratio of number of abnormal red blood cells in the subject to the total number of red blood cells in the subject can be determined by, for example, obtaining a blood sample, performing a blood smear, counting the number of abnormal red blood cells in the smear, counting the total number of red blood cells in the smear, and determining the ratio by dividing the number of abnormal red blood cells by the total number of red blood cells in the smear. The ratio of the number of red blood cells with basophilic stippling in the subject to the total number of red blood cells in the subject can be determined by, for example, obtaining a blood sample, performing a blood smear, counting the number of red blood cells with basophilic stippling in the smear, counting the total number of red blood cells in the smear, and determining the ratio by dividing the number of red blood cells with basophilic stippling by the total number of red blood cells in the smear. The ratio of the number of poikilocytic red blood cells in the subject to the total number of red blood cells in the subject can be determined by, for example, obtaining a blood sample, performing a blood smear, counting the number of poikilocytic red blood cells in the smear, counting the total number of red blood cells in the smear, and determining the ratio by dividing the number of poikilocytic red blood cells by the total number of red blood cells in the smear. The ratio of the number of schistocytes in the subject to the total number of red blood cells in the subject can be determined by, for example, obtaining a blood sample, performing a blood smear, counting the number of schistocytes in the smear, counting the total number of red blood cells in the smear, and determining the ratio by dividing the number of schistocytes by the total number of red blood cells in the smear. The ratio of the number of irregularly contracted red blood cells in the subject to the total number of red blood cells in the subject can be determined by, for example, obtaining a blood sample, performing a blood smear, counting the number of irregularly contracted red blood cells in the smear, counting the total number of red blood cells in the smear, and determining the ratio by dividing the number of irregularly contracted red blood cells by the total number of red blood cells in the smear.

### 7.7.6 Erythroid Response

The duration of the erythroid response can be calculated for a subject who achieves a response. The algorithm used to calculate the duration of response is as follows: (1) First Day of Response=the first day of the first 12-week interval showing response. Last Day of Response=last day of the last consecutive 129-week interval showing response. Date of Last Assessment=either the last visit date for subjects still on drug or the date of discontinuation for subjects who discontinued from the treatment. The duration of the erythroid response can be calculated as follows, depending on whether or not the response ends before the Date of Last Assessment: (1) a subject whose response does not continue to the end of a treatment period, the duration of response is not censored, and is calculated as: Response Duration=Last Day of Response—First Day of Response+1; (2) a subject who continues to exhibit an erythroid response at the end of a treatment period, the end date of the response is censored and duration of the response is calculated as: Response Duration=Date of Last Response Assessment—First Day of Response+1.

The time to the first erythroid response can be calculated as follows: the day from the first dose of study drug to the First Day of Response starts will be calculated using: Time to Response=First Day of Response—Date of First Study Drug+1.

### 7.7.7 Transfusion Burden

It is estimated that one unit of red blood cells contains approximately 200 mg of iron, while the body typically loses only 1.5 mg of iron per day. Transfusion burden in a subject treated according to the methods provided herein can be determined by determining the subject's transfusion requirement (i.e., the amount and the frequency of red blood cell transfusion). As a nonlimiting example, if a subject requiring transfusion of 2 units of red blood cells every 3 weeks achieves a reduction in frequency in transfusion to every 4 weeks upon treatment according to the methods provided herein, the subject has a 25% reduction in transfusion burden.

### 7.7.8 Assessment of Clinical Complications

Extramedullary hematopoietic (EMH) masses in a subject can be evaluated by assay(s) known to one skilled in the art, such as, for example, magnetic resonance imaging (MRI) and computed tomography scanning. In certain embodiments, EMH masses in a subject can be evaluated by MRI.

Splenomegaly can be evaluated by assay(s) known to one skilled in the art, such as, for example, magnetic resonance imaging (MRI).

Tricuspid regurgitant velocity (TRV) can be evaluated according to assay(s) known to one skilled in the art, such as, for example, echocardiography (ECHO).

Liver iron concentration in a subject can be evaluated by assay(s) known to one skilled in the art, such as, for example, magnetic resonance imaging (MRI).

### 7.7.9 Osteoporosis and Bone Mineral Density

Nonlimiting examples of osteoporosis symptoms include back pain, loss of height over time, stooped posture, easy bone fracturing, and decreased bone mineral density. Bone mineral density in a subject treated according to the methods provided herein can be determined by assay(s) known to one skilled in the art, such as, for example, by bone density scanning (also referred to as dual-energy x-ray absorptiometry (DXA or DEXA) or bone densitometry) and ultrasound. In certain embodiments, bone mineral density in a subject treated according to the methods provided herein is determined by DXA.

### 7.7.10 Skeletal Deformities

Skeletal deformities in subject treated according to the methods provided herein can be determined by assay(s) known to one skilled in the art, such as, for example, by x-ray and imaging techniques, such as, for example, magnetic resonance imaging (MRI) and computed tomography.

### 7.7.11 Bone Turnover

Various circulating markers of bone turnover can be used to diagnose bone disorders, such as low bone turnover. Circulating markers of bone turnover are markers of bone formation such as bone specific alkaline phosphatase (bAP), osteocalcin, procollagen type I C-terminal propeptide (PICP) and insulin-like growth factor-1 (IGF-1), some being markers of bone resorption such as pyridinoline, deoxypyridinoline, tartrate-resistant acid phosphatase (TRAP), TRAP type 5b, pyridinoline, deoxypyridinoline and procollagen type I C-terminal telopeptide (ICTP), serum or urine collagen cross-links (N-telopeptide or C-telopeptide), and 25 hydroxyvitamin D. Assays to measure the entire parathyroid hormone (PTH) molecule can also be used. The skilled artisan is aware of imaging methods allowing the assessment of bone mineral density (BMD), bone volume, trabecular bone volume, and trabecular thickness. See, e.g., Tilman B. Drueke and Sharon M. Moe, Disturbances of bone and mineral metabolism in chronic kidney disease: an international initiative to improve diagnosis and treatment, Nephrol Dial Transplant (2004) 19: 534-536; Okuno S, Inaba M., Biochemical markers of bone turnover. New aspect. Dialysis and bone metabolic marker, Clin Calcium. 2009 August; 19(8):1084-91; Herberth J, Monier-Faugere M C, Mawad H W, Branscum A J, Herberth Z, Wang G, Cantor T, Malluche H H, The five most commonly used intact parathyroid hormone assays are useful for screening but not for diagnosing bone turnover abnormalities in CKD-5 subjects, Clin Nephrol. 2009 July; 72(1):5-14; Lehmann G, Ott U, Kaemmerer D, Schuetze J, Wolf G., Bone histomorphometry and biochemical markers of bone turnover in subjects with chronic kidney disease Stages 3-5, Clin Nephrol. 2008 October; 70(4):296-305; Drneke T B., Is parathyroid hormone measurement useful for the diagnosis of renal bone disease?, Kidney Int. 2008 March; 73(6):674-6; Yamada S, Inaba M, Kurajoh M, Shidara K, Imanishi Y, Ishimura E, Nishizawa Y., Utility of serum tartrate-resistant acid phosphatase (TRACP5b) as a bone resorption marker in subjects with chronic kidney disease: independence from renal dysfunction., Clin Endocrinol (Oxf). 2008 August; 69(2):189-96. Epub 2008 Jan. 23. See also, Paul D. Miller, Diagnosis and Treatment of Osteoporosis in Chronic Renal Disease, 2009.

Another marker for monitoring bone resorption in CKD subjects with mild renal dysfunction is serum concentration of type I collagen N-telopeptide (S-NTX). See, e.g., Hamano T, Fujii N, Nagasawa Y, Isaka Y, Moriyama T, Okada N, Imai E, Horio M, Ito T., Serum NTX is a practical marker for assessing antiresorptive therapy for glucocorticoid treated subjects with chronic kidney disease., Bone. 2006 November; 39(5):1067-72. Epub 2006 Jun. 16.

Quantitative computed tomography (QCT) can also be used to determine bone turnover.

Markers, such as, for example, Runx2 and Alp can be evaluated to monitor the oseoblastic transition in a subject. Markers, such as, for example, Sm22-alpha can be evaluated to monitor vascular smooth muscle function and the levels of differentiated vascular smooth muscle cells.

### 7.7.12 Heart Size and Cardiac Hypertrophy

Heart size and cardiac hypertrophy can be determined by any method known to the skilled artisan, such as, for example, magnetic resonance imaging, electrocardiography, echocardiography, and noncontrast-enhanced cardiac computed tomography.

### 7.7.13 Quality of Life

To assess the quality of life for a subject treated according to the methods provided herein, the Short Form (36) Health Suvey (SF-26) and/or the Functional Assessment of Cancer Therapy-Anemia (FACT-An) can be utilized.

The SF-36 (Version 2.0) is a self-administered instrument consisting of 8 multi-item scales that assess 8 health domains: (1) Physical functioning (PF), 10 items from 3a to 3j; (2) Role-Physical (RP), 4 items from 4a to 4d; (3) Bodily Pain (BP), items 7 and 8; (4) General Health (GH), items 1 and 11a to 11d, (5) Vitality (VT), items 9a, 9e, 9g, and 9i; (6) Social functioning (SF), items 6 and 10; (7) Role-Emotional (RE), items 5a, 5b, and 5c; and (8) Mental Health (MH), 5 items 9b, 9c, 9d, 9f and 9h. Two overall summary scores can also be obtained: (1) a Physical Component Summary score (PCS); and (2) a Mental Component Summary score (MCS). Health domain scores, as well as the PCS and MCS scores, are transformed to norm based scores (mean of 50 and SD of 10), with higher scores indicating better health. The primary interests of the SF-36 are the health domain norm-based scores, and the PCS and MCS norm-based scores. Summary statistics (n, mean, standard deviation, median, minimum, and maximum) of health domain norm-based scores, PCS and MCS norm-based scores, as well as change from baseline in these norm-based scores can be assessed. Scoring for the SF-36 and methods to address missing values can be accomplished according to directions provided by the instrument developers.

Alternatively, FACT-An can be utilized to determine quality of life for a subject treated according to the methods provided herein. FACT-An is a 47-item, cancer-specific questionnaire consisting of a core 27-item general questionnaire (FACT-General, or FACT-G Total) measuring the four general domains of quality of life (physical, social/family, emotional and functional wellbeing). FACT-An scales are formatted on 1-4 pages, by subscale domain, for self-administration using a 5-point Likert rating scale (0=Not at all; 1=A little bit; 2=Somewhat; 3=Quite a bit; and 4=Very much). Scoring for the FACT instrument can be completed at the total scale level according to directions provided by the instrument developer. The FACT-G total score can be scored by summing the four domains within the general HRQoL instrument.

### 7.7.14 Common Terminology Criteria for Adverse Events (CTCAE, Version 4.0)

Grade 1 refers to mild adverse events. Specifically, Grade 1 refers to transient or mild discomfort. No limitation in activity and no medical intervention/therapy is required for Grade 1 adverse events. Grade 2 refers to moderate adverse events. Specifically, Grade 2 refers to mild to moderate limitation in activity. Some assistance may be needed, however, no or minimal medical intervention/therapy required for Grade 2 adverse events. Grade 3 refers to severe adverse events. Specifically, Grade 3 refers to marked limitation in activity. Some assistance is usually required and medical intervention/therapy is required, while hospitalization is possible for Grade 3 adverse events. Grade 4 refers to life-threatening adverse events. Specifically, Grade 4 refers to extreme limitation in activity, significant required assistance, significant required medical intervention/therapy, and hospitalization or hospice care is probable for Grade 4 adverse events. Grade 5 adverse event is death.

### 7.7.15 Hematocrit

A hematocrit measures the percentage of red blood cells in a given volume of whole blood and may be included as part of a standard complete blood count. The hematocrit is normally about 45% for men and about 40% for women. However, beta-thalassemia patients typically have a hematocrit lower than that normally seen. Thus, determination of the hematocrit in a beta-thalassemia patient being treated in accordance with the methods provided herein allows for the determination of the efficacy of such treatment.

### 7.7.16 Hemoglobin

Hemoglobin concentration can be determined according to an assay known to one skilled in the art. Beta-thalassemia patients typically have a hemoglobin concentration lower than that normally seen. Thus, determination of the hemoglobin concentration in a beta-thalassemia patient being treated in accordance with the methods provided herein allows for the determination of the efficacy of such treatment.

### 7.7.17 Screening Assays

Various ActRII polypeptide variants, or soluble ActRII polypeptide variants, may be tested for their ability to inhibit ActRII. In addition, compounds can be tested for their ability to inhibit ActRII. Once signaling inhibitors of ActRII activity are confirmed, these compounds can be used with the methods provided herein. ActRII can be ActRIIA or ActRIIB The assays below are described for ActRIIA but can be performed analogously for ActRIIB

For example, the effect of an ActRIIA polypeptide variant on the expression of genes involved in bone production or bone destruction may be assessed. This may, as needed, be performed in the presence of one or more recombinant ActRIIA ligand proteins (e.g., activin), and cells may be transfected so as to produce an ActRIIA polypeptide and/or variants thereof, and optionally, an ActRIIA ligand. Likewise, an ActRIIA polypeptide may be administered to a mouse or other animal, and one or more bone properties, such as density or volume may be assessed. The healing rate for bone fractures may also be evaluated. Dual-energy x-ray absorptiometry (DEXA) is a well-established, non-invasive, quantitative technique for assessing bone density in an animal. In humans central DEXA systems may be used to evaluate bone density in the spine and pelvis. These are the best predictors of overall bone density. Peripheral DEXA systems may be used to evaluate bone density in peripheral bones, including, for example, the bones of the hand, wrist, ankle and foot. Traditional x-ray imaging systems, including CAT scans, may be used to evaluate bone growth and fracture healing. In addition, bone density can be measured using quantitative computed tomography (qCT). The mechanical strength of bone may also be evaluated.

In certain aspects, provided herein is the use of ActRIIA polypeptides (e.g., soluble ActRIIA polypeptides) and activin polypeptides to identify compounds (agents) which are agonist or antagonists of the activin-ActRIIA signaling pathway. Compounds identified through this screening can be tested to assess their ability to modulate bone growth or mineralization in vitro. Optionally, these compounds can further be tested in animal models to assess their ability to modulate tissue growth in vivo.

There are numerous approaches to screening for therapeutic agents for modulating tissue growth by targeting activin and ActRIIA polypeptides. In certain embodiments, high-throughput screening of compounds can be carried out to identify agents that perturb activin or ActRIIA-mediated effects on bone. In certain embodiments, the assay is carried out to screen and identify compounds that specifically inhibit or reduce binding of an ActRIIA polypeptide to activin. Alternatively, the assay can be used to identify compounds that enhance binding of an ActRIIA polypeptide to activin. In a further embodiment, the compounds can be identified by their ability to interact with an activin or ActRIIA polypeptide.

A variety of assay formats will suffice and, in light of the present disclosure, those not expressly described herein will nevertheless be comprehended by one of ordinary skill in the art. As described herein, the test compounds (agents) used herein may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated herein include non-peptidyl organic molecules, peptides, polypeptides, peptidomimetics, sugars, hormones, and nucleic acid molecules. In a specific embodiment, the test agent is a small organic molecule having a molecular weight of less than about 2,000 daltons.

The test compounds can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatible crosslinkers or any combinations thereof.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between an ActRIIA polypeptide and activin.

Merely to illustrate, in an exemplary screening assay, the compound of interest is contacted with an isolated and purified ActRIIA polypeptide which is ordinarily capable of binding to activin. To the mixture of the compound and ActRIIA polypeptide is then added a composition containing an ActRIIA ligand. Detection and quantification of ActRIIA/activin complexes provides a means for determining the compound's efficacy at inhibiting (or potentiating) complex formation between the ActRIIA polypeptide and activin. The efficacy of the compound can be assessed by generating dose response curves from data obtained using various concentrations of the test compound. Moreover, a control assay can also be performed to provide a baseline for comparison. For example, in a control assay, isolated and purified activin is added to a composition containing the ActRIIA polypeptide, and the formation of ActRIIA/activin complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between the ActRIIA polypeptide and activin may be detected by a variety of techniques. For instance, modulation of the formation of complexes can be quantitated using, for example, detectably labeled proteins such as radiolabeled (e.g., 32P, 35S, 14C or 3H), fluorescently labeled (e.g., FITC), or enzymatically labeled ActRIIA polypeptide or activin, by immunoassay, or by chromatographic detection.

In certain embodiments, contemplated herein is the use of fluorescence polarization assays and fluorescence resonance energy transfer (FRET) assays in measuring, either directly or indirectly, the degree of interaction between an ActRIIA polypeptide and its binding protein. Further, other modes of detection, such as those based on optical waveguides (PCT Publication WO 96/26432 and U.S. Pat. No. 5,677,196), surface plasmon resonance (SPR), surface charge sensors, and surface force sensors, are compatible with many embodiments described herein.

Moreover, an interaction trap assay, also known as the “two hybrid assay,” can be used for identifying agents that disrupt or potentiate interaction between an ActRIIA polypeptide and its binding protein. See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, contemplated herein is the use of reverse two hybrid systems to identify compounds (e.g., small molecules or peptides) that dissociate interactions between an ActRIIA polypeptide and its binding protein. See for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368.

In certain embodiments, the subject compounds are identified by their ability to interact with an ActRIIA or activin polypeptide. The interaction between the compound and the ActRIIA or activin polypeptide may be covalent or non-covalent. For example, such interaction can be identified at the protein level using in vitro biochemical methods, including photo-crosslinking, radiolabeled ligand binding, and affinity chromatography (Jakoby W B et al., 1974, Methods in Enzymology 46: 1). In certain cases, the compounds may be screened in a mechanism based assay, such as an assay to detect compounds which bind to an activin or ActRIIA polypeptide. This may include a solid phase or fluid phase binding event. Alternatively, the gene encoding an activin or ActRIIA polypeptide can be transfected with a reporter system (e.g., β-galactosidase, luciferase, or green fluorescent protein) into a cell and screened against the library preferably by a high throughput screening or with individual members of the library. Other mechanism based binding assays may be used, for example, binding assays which detect changes in free energy. Binding assays can be performed with the target fixed to a well, bead or chip or captured by an immobilized antibody or resolved by capillary electrophoresis. The bound compounds may be detected usually using colorimetric or fluorescence or surface plasmon resonance.

In certain aspects, provided herein are methods and agents for modulating (stimulating or inhibiting) bone formation and increasing bone mass. Therefore, any compound identified can be tested in whole cells or tissues, in vitro or in vivo, to confirm their ability to modulate bone growth or mineralization. Various methods known in the art can be utilized for this purpose. In particular, the compounds can be tested for their ability to increase bone turnover.

For example, the effect of the ActRIIA or activin polypeptides or test compounds on bone or cartilage growth can be determined by measuring induction of Msx2 or differentiation of osteoprogenitor cells into osteoblasts in cell based assays (see, e.g., Daluiski et al., Nat Genet. 2001, 27(1):84-8; Hino et al., Front Biosci. 2004, 9:1520-9). Another example of cell-based assays includes analyzing the osteogenic activity of the subject ActRIIA or activin polypeptides and test compounds in mesenchymal progenitor and osteoblastic cells. To illustrate, recombinant adenoviruses expressing an activin or ActRIIA polypeptide can be constructed to infect pluripotent mesenchymal progenitor C3H10T1/2 cells, preosteoblastic C2C12 cells, and osteoblastic TE-85 cells. Osteogenic activity is then determined by measuring the induction of alkaline phosphatase, osteocalcin, and matrix mineralization (see, e.g., Cheng et al., J bone Joint Surg Am. 2003, 85-A(8): 1544-52).

Also provided herein are in vivo assays to measure bone or cartilage growth. For example, Namkung-Matthai et al., Bone, 28:80-86 (2001) discloses a rat osteoporotic model in which bone repair during the early period after fracture is studied. Kubo et al., Steroid Biochemistry & Molecular Biology, 68:197-202 (1999) also discloses a rat osteoporotic model in which bone repair during the late period after fracture is studied. Andersson et al., J. Endocrinol. 170:529-537 describe a mouse osteoporosis model in which mice are ovariectomized, which causes the mice to lose substantial bone mineral content and bone mineral density, with the trabecular bone losing roughly 50% of bone mineral density. Bone density could be increased in the ovariectomized mice by administration of factors such as parathyroid hormone. In certain aspects, fracture healing assays that are known in the art can be used. These assays include fracture technique, histological analysis, and biomechanical analysis, which are described in, for example, U.S. Pat. No. 6,521,750, which is incorporated by reference in its entirety for its disclosure of experimental protocols for causing as well as measuring the extent of fractures, and the repair process.

### 7.8 Combination Therapy

In certain embodiments, the methods provided herein are performed in combination with a second pharmaceutically active agent or therapy. Such combination therapy may be achieved by way of the simultaneous, sequential, or separate dosing of the individual components of the treatment. Additionally, when administered as a component of such combination therapy, the ActRII signaling inhibitor and the second pharmaceutically active agent or therapy may be synergistic, such that the daily dose of either or both of the components may be reduced as compared to the dose of either component that would normally be given as a monotherapy. Alternatively, when administered as a component of such combination therapy, the ActRII signaling inhibitor provided herein and the second pharmaceutically active agent or therapy may be additive, such that the daily dose of each of the components is similar or the same as the dose of either component that would normally be given as a monotherapy.

In certain embodiments, the ActRII signaling inhibitor provided herein is administered on the same day as a second pharmaceutically active agent or therapy. In certain embodiments, the ActRII signaling inhibitor is administered one, two, three, or more days before a second pharmaceutically active agent or therapy. In certain embodiments, the ActRII signaling inhibitor is administered one, two, three or more days after a second pharmaceutically active agent or therapy. In certain embodiments, the ActRII signaling inhibitor is administered within one, two, three or more weeks of a second pharmaceutically active agent or therapy.

In certain embodiments, the second pharmaceutically active agent or therapy is an active agent or therapy, respectively, used to treat beta-thalassemia. Non-limiting examples or pharmaceutically active agents or therapies used to treat beta-thalassemia include red blood cell transfusion, iron chelation therapy, such as, for example, deferoxamine, deferiprone, and/or deferasirox, fetal hemoglobin inducing agents, such as, for example, hydroxyurea, and hematopoietic stem cell transplantation.

### 7.9 Pharmaceutical Compositions

In certain embodiments, ActRII signaling inhibitors (e.g., ActRII polypeptides) are formulated with a pharmaceutically acceptable carrier for use with the methods described herein. For example, an ActRII polypeptide can be administered alone or as a component of a pharmaceutical formulation (therapeutic composition). The subject compounds may be formulated for administration in any convenient way for use in human or veterinary medicine. ActRII can be ActRIIA or ActRIIB

In a preferred embodiment, the ActRII signaling inhibitor is formulated for subcutaneous administration.

In another preferred embodiment, the ActRII signaling inhibitor is packaged in a container as a sterile, preservative-free lyophilized powder or cake. In certain embodiments, the container comprises 25 mg of the ActRII signaling inhibitor. In certain embodiments, the container comprising 25 mg of the ActRII signaling inhibitor comprises a total of 37.5 mg of protein. In certain embodiments, ActRII signaling inhibitor in the container comprising 25 mg of the ActRII signaling inhibitor is reconstituted with 0.68 mL of water for injection. In certain embodiments, the container comprises 75 mg of the ActRII signaling inhibitor. In certain embodiments, the container comprising 75 mg of the ActRII signaling inhibitor comprises a total of 87.5 mg of protein. In certain embodiments, ActRII signaling inhibitor in the container comprising 75 mg of the ActRII signaling inhibitor is reconstituted with 1.6 mL of water for injection. In certain embodiments, the ActRII signaling inhibitor in the container is reconstituted with a volume of water for injection, such that the final concentration of the reconstituted ActRII signaling inhibitor in the water for injection is 50 mg/mL with a pH of approximately 6.5. In certain embodiments, the ActRII signaling inhibitor is administered to a subject within 10 hours of reconstitution. In certain embodiments, the container comprises the ActRII signaling inhibitor at a concentration of 50 mg/mL in a 10 mM citrate buffer-based solution, wherein the 10 mM citrate-buffer based solution comprises 10 mM citrate, pH 6.5, 9% sucrose, and 0.02% polysorbate 80. In certain embodiments, the container is stored at between 2° C. and 8° C. In certain embodiments, the container is stored at between 2° C. and 8° C. for 18 months. In certain embodiments, the container is a 3 mL glass vial with a gray butyl coated stopper. In certain embodiments, the container is a 3 mL glass vial with a gray rubber stopper. In certain embodiments, the rubber stopper is secured in place by a crimped aluminum flip cap with a colored plastic button. In certain embodiments, the 3 mL glass vial comprises 25 mg of the ActRII signaling inhibitor and the the colored plastic button is red. In certain embodiments, 3 mL glass vial comprises 75 mg of the ActRII signaling inhibitor and the the colored plastic button is white.

In a specific embodiment, the ActRII signaling inhibitor is packaged in a container as a sterile, preservative-free lyophilized powder or cake. In a specific embodiment, the container comprises 50 mg/mL of ActRII signaling inhibitor in 10 mM citrate buffer pH 6.5. In a specific embodiment, the container comprises 56 mg of ActRII signaling inhibitor, 0.19 mg of citric acid monohydrate, 3.03 mg of tri-sodium citrate dehydrate, 0.24 mg of polysorbate 80, and 100.80 mg of sucrose.

In certain embodiments, the therapeutic methods provided herein include administering the composition (comprising an ActRII signaling inhibitor) systemically, or locally as an implant or device. When administered, the therapeutic composition for uses provided herein is in a pyrogen-free, physiologically acceptable form. Therapeutically useful agents other than the ActRII signaling inhibitors which may also optionally be included in the composition as described above, may be administered simultaneously or sequentially with the subject compounds (e.g., ActRII polypeptides, such as ActRIIA and/or ActRIIB polypeptides (see, Section 7.6)).

Typically, ActRII signaling inhibitors will be administered parenterally. In a preferred embodiment, the ActRII signaling inhibitor will be administered subcutaneously. Pharmaceutical compositions suitable for parenteral administration may comprise one or more ActRII polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions for use in the methods described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions described herein may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the compounds described herein (e.g., ActRII polypeptides, such as ActRIIA and/or ActRIIB polypeptides (see, Section 7.6) as described in Section 7.3.2 and Table 1 and Table 2, above).

In certain embodiments, the ActRII signaling inhibitor is substantially pure in a pharmaceutical composition. Specifically, at most 20%, 10%, 5%, 2.5%, 1%, 0.1%, or at most 0.05% of the compounds in the pharmaceutical composition are compounds other than the ActRII signaling inhibitor and the pharmaceutical acceptable carrier.

In certain embodiments, the ActRII signaling inhibitor is administered at room temperature to a patient (e.g., as set forth in Section 7.5) according to a method provided herein.

### 8. EXAMPLES

### 8.1 Example 1: A Phase 3, Double Blind, Randomized, Placebo Controlled Multicenter Study to Determine the Efficacy and Safety of MACTRIIB-FC in Adults with Transfusion-Dependent Beta Thalassemia

This example provides an overview of a phase 3, double blind, randomized, placebo controlled multicenter study to determine the efficacy and safety of ActRIIB-hFc (SEQ ID NO:25) in adults who require regular red blood cell transfusions due to beta-thalassemia. The indication for the phase 3 study is adults with transfusion-dependent beta-thalassemia, with a documented diagnosis of beta-thalassemia or hemoglobin E/beta-thalassemia, excluding hemoglobin S/beta-thalassemia.

### 8.1.1 Objectives

The primary objective of the phase 3 study is to determine the proportion of subjects with erythroid response, defined as >33% reduction in transfusion burden (units red blood cells over time) over 12 consecutive weeks, after the minimum of 6 months of treatment compared to the 12-week interval prior to randomization for ActRIIB-hFc (SEQ ID NO:25) best supportive care (BSC) versus placebo plus BSC.

The secondary objectives of the phase 3 study include: (1) to evaluate the safety and immunogenicity of ActRIIB-hFc (SEQ ID NO:25) versus placebo; (2) To evaluate the effect of ActRIIB-hFc (SEQ ID NO:25) on the proportion of subjects who are transfusion-free for weeks versus placebo; (3) to evaluate the effect of ActRIIB-hFc (SEQ ID NO:25) on the change in liver iron concentration (LIC) versus placebo; (4) to evaluate the effect of ActRIIB-hFc (SEQ ID NO:25) treatment on quality of life (QoL) measures (e.g., new non-transfusion-dependent-specific PRO, SF-36) versus placebo; (5) to evaluate the effect of ActRIIB-hFc (SEQ ID NO:25) on osteoporosis (bone mineral density) versus placebo; (6) to evaluate the use of ActRIIB-hFc (SEQ ID NO:25) on health resource utilization; (7) to evaluate the effect of ActRIIB-hFc (SEQ ID NO:25) on the mean percentage change in transfusion burden versus placebo plus BSC, over the same 12-week period used in the primary endpoint analysis, compared to the 12-week interval prior to randomization; (8) to evaluate the duration of reduction in transfusion burden or transfusion independence; (9) to evaluate the time to erythroid response; (10) to evaluate effect of ActRIIB-hFc (SEQ ID NO:25) on change in serum ferritin; (11) to evaluate effect of ActRIIB-hFc (SEQ ID NO:25) on change in cardiac iron overload; and (12) to evaluate the population pharmacokinetics (PK) of ActRIIB-hFc (SEQ ID NO:25) in subjects with beta-thalassemia.

The exploratory objectives are: (1) to examine the relationship of baseline and change in serum GDF11 with response to treatment with ActRIIB-hFc (SEQ ID NO:25); and (2) to examine the effect of ActRIIB-hFc (SEQ ID NO:25) on change in fetal hemoglobin (HbF).

### 8.1.2 Study Design

This example presents a phase 3, double-blind, randomized, placebo-controlled, multicenter study to determine the efficacy and safety of ActRIIB-hFc (SEQ ID NO:25) (ACE-536) plus best supportive care versus best supportive care (BSC) in adults with transfusion dependent beta-thalassemia. The study is divided into (i) the Screening Period, (ii) double-blind Treatment Period, (iii) open-label Extension Period, and (iv) Follow-up Period.

Patient eligibility is determined during the Screening Period to determine eligibility, which is within 28 days prior to Dose 1 Day 1. Patients are stratified based on the following factors: (1) transfusion burden at baseline, wherein high transfusion burden is 15 RBC units in the 24 weeks prior to randomization, and wherein low transfusion burden is 7-14 RBC units in the 24 weeks prior to randomization; and (2) geographical region.

During the treatment period, eligible subjects will be randomized to either the experimental arm (ActRIIB-hFc (SEQ ID NO:25)) plus BSC or control arm (placebo) plus BSC at a 2:1 ratio. The double-blind Treatment Period is considered the first 48 weeks following Study Day 1 (i.e., Dose 1 Day 1), independent of dose delays. Treatment with ActRIIB-hFc (SEQ ID NO:25) for each subject begins on Study Day 1. Subjects will begin treatment at a starting dose level of about 0.8 mg/kg of ActRIIB-hFc (SEQ ID NO:25) administered by subcutaneous (SC) injection once every 3 weeks for 48 weeks. The dose of the ActRIIB-hFc (SEQ ID NO:25) may be titrated up to a maximum of about 1.25 mg/kg.

Subjects can be dose-escalated stepwise from the starting dose about 0.8 mg/kg of ActRIIB-hFc (SEQ ID NO:25) to about 1 mg/kg of ActRIIB-hFc (SEQ ID NO:25) and then to about 1.25 mg/kg of ActRIIB-hFc (SEQ ID NO:25) during the treatment period as well as during the extension period unless dose modification is required. Dose escalation will be based on transfusion frequency during the previous two cycles (i.e., the previous 6 weeks).

The dose of ActRIIB-hFc (SEQ ID NO:25) or placebo for each subject may be delayed and/or reduced following the dose modification guidelines, as detailed in Table 1 and Table 2, above.

All subjects will have the option to enroll in an open-label Extension Period and receive ActRIIB-hFc (SEQ ID NO:25) upon completion of the 48-week double-blind Treatment Period, at the Investigator's discretion. The open-label Extension Period will last 96 weeks (i.e., 2 years) and is subjected to dose escalation, dose modifications, dose delay and reductions as described in Table 1 and Table 2, above. The Extension Period may be prolonged based on the evolving safety data.

Subjects who complete the open-label Extension Period or who do not enroll in the open-label Extension Period or who discontinue early from treatment will proceed to the post-treatment Follow-up Period. The Follow-up Period will last 12 weeks following the subject's last dose of study drug.

### 8.1.2.1 Subject Population

The subject population consists of subjects diagnosed with transfusion dependent beta-thalassemia, including hemoglobin E/beta-thalassemia, aged 18 years and who are transfusion-dependent. Transfusion dependence is defined as regular transfusions of 7 red blood cell units per 24 weeks with no transfusion-free period 35 days in the 24 weeks prior to randomization. In certain aspects, transfusion dependence is defined as regular transfusions of >6 red blood cell units per 24 weeks with no transfusion-free period 35 days in the 24 weeks prior to randomization. In certain aspects, transfusion dependence is defined as regular transfusions of >5 red blood cell units per 24 weeks with no transfusion-free period 35 days in the 24 weeks prior to randomization.

### 8.1.2.2 Length of Study

Study participation for each subject is approximately up to 160 weeks (40 months), including: up to a 4 weeks (1 month) screening period, 48 weeks (12 month) placebo-controlled treatment period, followed by an open-label Extension Period which will last approximately up to 96 weeks (2 years). Post-treatment follow-up period will last 12 weeks (3-month) post-last dose.

The End of Treatment for each individual subject is defined as the date of the last visit in the Treatment Period or in the Open-Label Extension Period, whichever is the later date. The End of Study is defined as the date of the last visit of each individual subject in the Treatment Period or in the Open-Label Extension Period, whichever is the later date, and completes the 12 weeks of the Post-Treatment Follow-Up Period. The End of Trial is defined as either the date of the last visit of the last subject to complete the post-treatment follow-up, or the date of receipt of the last data point from the last subject that is required for primary, secondary, and/or exploratory analyses, whichever is the later date, as pre-specified in the protocol and/or Statistical Analysis Plan.

### 8.1.2.3 Study Treatment

ActRIIB-hFc (SEQ ID NO:25) will be provided as a lyophilized powder, which will be administered to the subject after reconstitution as a subcutaneous (SC) injection to subject. Subcutaneous injections will be given in the upper arm, abdomen, or thigh, every 3 weeks during the Treatment Period and during the open-label Extension Period, if applicable. Subjects will start ActRIIB-hFc (SEQ ID NO:25) at about 0.8 mg/kg dose level and can be dose escalated up to a maximum of about 1.25 mg/kg (see, Table 1 and Table 2, above).

Placebo (normal saline) will be administered to the subject as a subcutaneous (SC) injection to subjects by the study staff at the clinical site. Subcutaneous injections will be given in the upper arm, abdomen, or thigh, every 3 weeks during the Treatment Period.

### 8.1.2.4 Overview of Key Efficacy Assessments

The primary efficacy assessment is the proportion of subjects with 33% reduction in transfusion burden (units red blood cells over time) over 12 consecutive weeks, evaluated after a minimum of 6 months of treatment, compared to the 12-week interval prior to randomization for ActRIIB-hFc (SEQ ID NO:25) versus placebo plus BSC.

The secondary efficacy assessment includes: (1) proportion of subjects who are transfusion-free for ≥8 weeks during treatment; (2) change in liver iron concentration (LIC, mg/g dry weight) as determined by magnetic resonance imaging (MRI); (3) change in quality of life (QoL; using TranQoL); and (4) change in mean daily dose of iron chelation therapy.

Other efficacy assessments will include: (1) total hip, and lumbar spine bone mineral density as determined by DXA; (2) healthcare resource utilization; (3) percentage change in transfusion burden using the same 12-week period as the primary endpoint; (4) duration of reduction in transfusion burden or transfusion independence; (5) time to erythroid response; (6) change in serum ferritin; and (7) change in cardiac iron overload as determined by MRI; change in QoL as determined by SF-36.

### 8.1.2.5 Overview of Key Safety Assessments

All patients will be assessed for safety by monitoring AEs, clinical laboratory tests, vital signs, electrocardiogram (ECG), cardiac Doppler, anti-drug antibody (ADA) testing, and ECOG performance status.

### 8.1.2.6 Overview of Key Exploratory Assessments

The ability of treatment of a subject with the ActRIIB-hFc (SEQ ID NO:25) to decrease serum GDF11 concentrations/levels and/or to increase fetal hemoglobin concentrations/levels in the subject will be evaluated.

### 8.2 Example 2: A Phase 3, Double Blind, Randomized, Placebo Controlled Multicenter Study to Determine the Efficacy and Safety of ACTRIIB-HFC (SEQ ID NO:25) in Adults with Non-Transfusion-Dependent Beta Thalassemia

This example provides an overview of a phase 3, double blind, randomized, placebo controlled multicenter study to determine the efficacy and safety of ActRIIB-hFc (SEQ ID NO:25) in adults with non-transfusion-dependent beta-thalassemia. The indication for the phase 3 study is adults with non-transfusion-dependent beta-thalassemia, with a documented diagnosis of beta-thalassemia or hemoglobin E/beta-thalassemia.

### 8.2.1 Objectives

The primary objective of the phase 3 study is to determine the effects of ActRIIB-hFc (SEQ ID NO:25) in subjects diagnosed with non-transfusion-dependent beta-thalassemia, with a documented diagnosis of beta-thalassemia or hemoglobin E/beta-thalassemia, aged 18 years and who received 0 to 6 red blood cell units during the 24-week period prior to randomization, with a mean baseline hemoglobin level <10.0 g/dL. In certain aspects, the subjects have received 0 to 5 red blood cell units during the 24-week period prior to randomization.

The secondary objectives of the phase 3 study include: (1) to evaluate the safety and immunogenicity of ActRIIB-hFc (SEQ ID NO:25) versus placebo; (2) to evaluate the effect of ActRIIB-hFc (SEQ ID NO:25) on the change in liver iron concentration (LIC) versus placebo; (3) to evaluate the effect of ActRIIB-hFc (SEQ ID NO:25) treatment on quality of life (QoL) measures (e.g., new non-transfusion-dependent-specific PRO, SF-36) versus placebo; (4) to evaluate the effect of ActRIIB-hFc (SEQ ID NO:25) on improvement of complications of thalassemia, when present, including extramedullary hematopoietic masses, leg ulcers, splenomegaly, pulmonary hypertension (PAH; measured by tricuspid regurgitation velocity (TRV)) and osteoporosis (measured by bone mineral density) versus placebo; (5) to evaluate the change in mean daily dose of iron chelation therapy (ICT) used in the last 4 weeks of treatment versus the 4 week period prior to randomization versus placebo; (6) to evaluate the effect of ActRIIB-hFc (SEQ ID NO:25) on change in serum ferritin; (7) to evaluate the effect of ActRIIB-hFc (SEQ ID NO:25) on the mean change in hemoglobin level from baseline over a continuous 12-week interval during treatment versus placebo; (8) to evaluate the duration of erythroid response; and (9) to evaluate population pharmacokinetics (PK) of ActRIIB-hFc (SEQ ID NO:25) in subjects with beta-thalassemia.

The exploratory objectives are: (1) to examine the relationship of baseline and change in serum GDF11 with response to treatment with ActRIIB-hFc (SEQ ID NO:25); (2) to examine the effect of ActRIIB-hFc (SEQ ID NO:25) on change in fetal hemoglobin (HbF); (3) to examine the in vivo efficacy of ActRIIB-hFc (SEQ ID NO:25) on RBC quality; and (4) to examine the effect of ActRIIB-hFc (SEQ ID NO:25) on health resource utilization.

### 8.2.2 Study Design

This example presents a phase 3, double-blind, randomized, placebo-controlled, multicenter study to determine the efficacy and safety of ActRIIB-hFc (SEQ ID NO:25) (ACE-536) plus best supportive care versus best supportive care in adults with non-transfusion dependent beta-thalassemia. The study is divided into (i) the Screening Period, (ii) double-blind Treatment Period, (iii) open-label Extension Period, and (iv) Follow-up Period.

Patient eligibility is determined during the Screening Period to determine eligibility, which is within 28 days prior to randomization. Patients are stratified based on the following factors: (1) baseline hemoglobin level 8.5 g/dL or <8.5 g/dL), and (2) ICT use.

During the treatment period, eligible subjects will be randomized to either the experimental arm (ActRIIB-hFc (SEQ ID NO:25)) plus BSC or control arm (placebo) plus BSC at a 2:1 ratio. The double-blind Treatment Period is considered the first 48 weeks following Study Day 1 (i.e., Dose 1 Day 1), independent of dose delays. Treatment with ActRIIB-hFc (SEQ ID NO:25) for each subject begins on Study Day 1. Subjects will begin treatment at a starting dose level of about 0.8 mg/kg of ActRIIB-hFc (SEQ ID NO:25) administered by subcutaneous (SC) injection once every 3 weeks for 48 weeks. The dose of the ActRIIB-hFc (SEQ ID NO:25) may be titrated up to a maximum of about 1.25 mg/kg.

Subjects can be dose-escalated stepwise from the starting dose about 0.8 mg/kg of ActRIIB-hFc (SEQ ID NO:25) to about 1 mg/kg of ActRIIB-hFc (SEQ ID NO:25) and then to about 1.25 mg/kg of ActRIIB-hFc (SEQ ID NO:25) during the treatment period as well as during the extension period unless dose modification is required. Dose escalation will be based on transfusion frequency during the previous two cycles (i.e., the previous 6 weeks).

The dose of ActRIIB-hFc (SEQ ID NO:25) or placebo for each subject may be delayed and/or reduced following the dose modification guidelines, as detailed in Table 1 and Table 2, above.

All subjects will have the option to enroll in an open-label Extension Period and receive ActRIIB-hFc (SEQ ID NO:25) upon completion of the 48-week double-blind Treatment Period, at the Investigator's discretion. The open-label Extension Period will last 96 weeks (i.e., 2 years) and is subjected to dose escalation, dose modifications, dose delay and reductions as described in Table 1 and Table 2, above. The Extension Period may be prolonged based on the evolving safety data.

Subjects who complete the open-label Extension Period or who do not enroll in the open-label Extension Period or who discontinue early from treatment will proceed to the post-treatment Follow-up Period. The Follow-up Period will last 12 weeks following the subject's last dose of study drug.

### 8.2.2.1 Subject Population

The subject population consists of subjects diagnosed with non-transfusion dependent beta-thalassemia, with a documented diagnosis of β-thalassemia or hemoglobin E/beta-thalassemia, aged 18 years and who received 0 to 6 RBC units during the 24-week period prior to randomization, with a mean baseline hemoglobin level <10.0 g/dL. In certain aspects, the subject has received 0 to 5 RBC units during the 24-week period prior to randomization.

### 8.2.2.2 Length of Study

Study participation for each subject is approximately up to 160 weeks (40 months), including: up to a 4 weeks (1 month) screening period, 48 weeks (12 month) placebo-controlled treatment period, followed by an open-label Extension Period which will last approximately up to 96 weeks (2 years). Post-treatment follow-up period will last 12 weeks (3-month) post-last dose.

The End of Treatment for each individual subject is defined as the date of the last visit in the Treatment Period or in the Open-Label Extension Period, whichever is the later date. The End of Study is defined as the date of the last visit of each individual subject in the Treatment Period or in the Open-Label Extension Period, whichever is the later date, and completes the 12 weeks of the Post-Treatment Follow-Up Period. The End of Trial is defined as either the date of the last visit of the last subject to complete the post-treatment follow-up, or the date of receipt of the last data point from the last subject that is required for primary, secondary, and/or exploratory analyses, whichever is the later date, as pre-specified in the protocol and/or Statistical Analysis Plan.

### 8.2.2.3 Study Treatment

ActRIIB-hFc (SEQ ID NO:25) will be provided as a lyophilized powder, which will be administered to the subject after reconstitution as a subcutaneous (SC) injection to subject. Subcutaneous injections will be given in the upper arm, abdomen, or thigh, every 3 weeks during the Treatment Period and during the open-label Extension Period, if applicable. Subjects will start ActRIIB-hFc (SEQ ID NO:25) at about 0.8 mg/kg dose level and can be dose escalated up to a maximum of about 1.25 mg/kg (see, Table 1 and Table 2, above).

Placebo (normal saline) will be administered to the subject as a subcutaneous (SC) injection to subjects by the study staff at the clinical site. Subcutaneous injections will be given in the upper arm, abdomen, or thigh, every 3 weeks during the Treatment Period.

### 8.2.2.4 Overview of Key Efficacy Assessments

The primary efficacy assessment is the proportion of subjects who display an erythroid response: the subjects are transfusion-free and have a hemoglobin increase from baseline of 1.0 g/dL as measured by the mean of hemoglobin values over a continuous 12-week interval, after a minimum of 6 months of treatment versus placebo plus BSC. Such assessment requires at least 2 hemoglobin measurements, by central laboratory, performed at one week intervals, over a 4 week period.

The secondary efficacy assessment includes: (1) change in liver iron concentration (LIC, mg/g dry weight) as determined by magnetic resonance imaging (MRI); (2) change in quality of life (QoL; new non-transfusion-dependent-specific patient-reported outcome (PRO)); (3) change in daily dose of iron chelation therapy; (4) change in serum ferritin concentration; (5) mean change in hemoglobin from baseline over 12 weeks; (6) duration of mean hemoglobin increase from baseline 1.0 g/dL, in absence of transfusions, in the absence of transfusions; (7) population pharmacokinetic parameters and exposure-response relationships; and (8) change in one or more of the following morbidities: (i) extramedullary hematopoietic mass volume as determined by Mill; (ii) leg ulcer size; (iii) spleen volume as determined by Mill; (iv) TRV as measured by echocardiogram; and (v) bone mineral density as measured by DXA.

### 8.2.2.5 Overview of Key Safety Assessments

All patients will be assessed for safety by monitoring AEs, clinical laboratory tests, vital signs, electrocardiogram (ECG), cardiac Doppler, anti-drug antibody (ADA) testing, and ECOG performance status.

### 8.2.2.6 Overview of Key Exploratory Assessments

The ability of treatment of a subject with the ActRIIB-hFc (SEQ ID NO:25) to decrease serum GDF11 concentrations/levels and/or to increase fetal hemoglobin concentrations/levels in the subject will be evaluated. In addition, the impact of treatment of a subject with ActRIIB-hFc (SEQ ID NO:25) on red blood cell quality will also be evaluated. Finally, the impact of treatment of a subject with ActRIIB-hFc (SEQ ID NO:25) on Health Resource Utilization by the subject will also be evaluated.

### 8.3 Example 3: ACTRIIb-HFC (SEQ ID NO:25) Signaling Inhibitor Increases Hemoglobin and Decreases Transfusion Burden and Liver Iron Concentration in Adults with Beta Thalassemia

### 8.3.1 Introduction

ActRIIB-hFc (SEQ ID NO:25), a fusion protein containing modified activin receptor, is being developed for the treatment of beta-thalassemia. In beta-thalassemia, anemia and complications arise due to ineffective erythropoiesis driven by excess alpha-globin. ActRIIB-hFc (SEQ ID NO:25) binds to GDF11 and other ligands in the TGF-β superfamily to promote late-stage erythroid differentiation. Non-clinical and clinical studies demonstrated ActRIIB-hFc (SEQ ID NO:25) was well-tolerated and corrected effects of ineffective erythropoiesis (Suragani R, Blood 2014, Attie K, Am J Hematol 2014).

This example presents data from an ongoing, phase 2, multicenter, open-label, dose-finding study to evaluate ActRIIB-hFc (SEQ ID NO:25) in adults with transfusion-dependent or non-transfusion-dependent beta-thalassemia. Efficacy outcomes include hemoglobin (Hb) increase in patients with non-transfusion-dependent beta-thalassemia, reduced RBC transfusion burden in patients with transfusion-dependent beta-thalassemia, and liver iron concentration (LIC) by magnetic resonance imaging (MRI).

### 8.3.2 Methods

Inclusion criteria included humans 18 years old with anemia, defined as being transfusion-dependent or as being non-transfusion-dependent with baseline Hb<10.0 g/dL. ActRIIB-hFc (SEQ ID NO:25) was administered subcutaneously every three weeks for up to 5 doses with a 2-month follow-up study. Sequential cohorts (n=6 each) were dosed at 0.2, 0.4, 0.6, 0.8, 1.0, and 1.25 mg/kg. An expansion cohort (n=30) is ongoing; patients who complete study may enroll in an ongoing 12-month extension study.

### 8.3.3 Results

Preliminary data (as of date) were available for 35 patients (25 non-transfusion-dependent patients and 10 transfusion-dependent patients) treated for 3 months. The median age of the patients was 35.0 years old (20-57 years old) and 86% of the patients had prior splenectomy. The mean (SD) baseline Hb for non-transfusion-dependent patients was 8.4 (±0.9) g/dL. Transfusion burden for transfusion-dependent patients prior to treatment ranged from 6 to 8 units/12 weeks. Twenty patients were on stable iron chelation therapy (ICT) at baseline.

The mean (SD) maximum increase in Hb for non-transfusion-dependent patients treated with 0.8-1.25 mg/kg of ActRIIB-hFc (SEQ ID NO:25; n=8) was 1.7 g/dL, compared with 1.2 g/dL in patients treated with 0.2-0.6 mg/kg of ActRIIB-hFc (SEQ ID NO:25; n=17). Three out of eight (38%) patients in the higher dose groups had a mean increase in Hb >1.5 g/dL, which was sustained for weeks (mean duration time of 9 weeks), compared with zero out of seventeen patients in the lower dose groups. All nine transfusion-dependent patients treated with 0.8-1.25 mg/kg of ActRIIB-hFc (SEQ ID NO:25) had >20% decrease in transfusion burden over 12 weeks on treatment compared with 12 weeks pre-treatment (mean 72%, range 43-100%).

In transfusion-dependent patients, the mean baseline liver iron concentration was 7.4 mg Fe/g dry weight (n=9) despite iron chelation therapy, and the mean reduction in liver iron concentration was 16.3% by week 16 of ActRIIB-hFc (SEQ ID NO:25) treatment. In non-transfusion-dependent patients with baseline liver iron concentration of 5 mg/g dry weight, the mean reduction in liver iron concentration was 18.2% in those dosed with 0.6-1.25 mg/kg of ActRIIB-hFc (SEQ ID NO:25; n=5), compared with 7.0% in those dosed with 0.2-0.4 mg/kg of ActRIIB-hFc (SEQ ID NO:25; n=5). In non-transfusion-dependent patients with baseline liver iron concentration of <5 mg/kg dry weight, mean change in liver iron concentration was −1.2% (n=10). Three patients with long-standing leg ulcers at baseline (two non-transfusion-dependent patients and one transfusion-dependent patient) had substantial healing within 4-6 weeks after initiating ActRIIB-hFc (SEQ ID NO:25) treatment.

ActRIIB-hFc (SEQ ID NO:25) was generally well tolerated, with no related serious adverse events reported to date. The most frequent related adverse events included bone pain, headache, myalgia, pain in extremity, and asthenia. No notable changes in platelets or white blood cells were observed.

### 8.3.4 Conclusions

ActRIIB-hFc (SEQ ID NO:25) administered to patients subcutaneously every 3 weeks for up to 5 doses was generally safe and well-tolerated, increased Hb levels in non-transfusion-dependent beta-thalassemia patients, and decreased transfusion requirement in transfusion-dependent beta-thalassemia patients. Both transfusion-dependent and non-transfusion-dependent patients had substantial decreases in liver iron concentration during treatment and healing of leg ulcers occurred in three out of three patients. ActRIIB-hFc (SEQ ID NO:25) is a promising therapy for patients with either transfusion-dependent or non-transfusion-dependent beta-thalassemia.

### 8.4 Example 4: ACTRIIb-HFC (SEQ ID NO:25) Signaling Inhibitor Increases Hemoglobin and Decreases Transfusion Burden and Liver Iron Concentration in Adults with Beta Thalassemia (Continued)

### 8.4.1 Introduction

See the Introduction (Section 8.3.1) and Materials and Methods (Section 8.3.2). This example presents additional data from Section 8.3, obtained at a later date in the Phase 2 study. Briefly, dose escalation cohorts (total of 35 patients) received between 0.2 to 1.25 mg/kg (3-6 patients per cohort). Specifically, the doses for the dose escalation cohorts were 0.2 (6 patients); 0.4 (6 patients); 0.6 (6 patients); 0.8 (6 patients); 1.0 (6 patients); and 1.25 mg/kg (5 patients). An expansion cohort started at 0.8 mg/kg (4 patients; dose level increased to 1.0 mg/kg in 2 patients; with potential dosing up to 1.25 mg/kg). ActRIIB-hFc (SEQ ID NO:25) was administered subcutaneously every 3 weeks for 3 months. An extension study is ongoing for an additional 12 months of treatment. Primary efficacy endpoints were as follows. For non-transfusion dependent patients (NTD; less than 4 U/8 weeks, hemoglobin of less than 10 g/dl): Hb increase of ≥1.5 g/dL for ≥2 weeks; for transfusion dependent patients (TD; equal to or more than 4 U/8 weeks confirmed over 6 months): Transfusion burden decrease ≥20% over 12 weeks. Secondary efficacy endpoints were liver iron concentration (measured by MRI), serum ferritin, and biomarkers of erythropoiesis.

### 8.4.2 Results

Preliminary data (as of date) were available for 39 patients (25 non-transfusion-dependent patients and 14 transfusion-dependent patients) treated with ActRIM-hFc (SEQ ID NO:25) treatment for 3 months, and 4 patients were further treated with ActRIIB-hFc (SEQ ID NO:25) treatment during a subsequent 12-month extension period. The median age of the patients was 40.0 years old (20-57 years old), 49% were male, and 32% of the patients had prior splenectomy. The mean (SD) baseline Hb for non-transfusion-dependent patients (NTD) was 8.3 (±0.9) g/dL. The mean liver iron concentration (by Mill) in NTD was 5.8±3.8 mg/g dw. The transfusion dependent patients received a mean of 7.3 (±0.9) RBC units/12 weeks and had a mean liver iron concentration (LIC) of 5.2(±5.7) mg/g dw. Regarding LIC, the clinical goal is to maintain LIC below 5 mg/g dw in non-transfusion dependent patient and below 7 mg/g dw in transfusion dependent patients.

Four out of eight (50%) non-transfusion dependent patient in the higher dose groups (i.e., 0.8 to 1.25 mg/kg) had a mean increase in Hb >1.5 g/dL, which was sustained for 2 weeks, compared with zero out of seventeen patients in the lower dose groups (i.e., 0.2 to 0.6 mg/kg). Three out of eight (38%) non-transfusion dependent patient in the higher dose groups (i.e., 0.8 to 1.25 mg/kg) had a mean increase in Hb >1.5 g/dL, which was sustained for weeks, compared with zero out of seventeen patients in the lower dose groups (i.e., 0.2 to 0.6 mg/kg).

Ten out of ten (100%) non-transfusion dependent patients with a baseline LIC <5 mg/g dw maintained LIC <5 mg/g dw. In three patients, LIC dropped by between about 0.5 mg/g dw and about 2 mg/g dw in the course of a 4 month treatment period. In two patients, LIC increased by between about 0.5 mg/g dw and about 1.0 mg/g dw over, and in five patients, LIC remained essentially unchanged over the 4 month treatment period. Two patients receiving iron chelators saw their LIC decrease by 0.5 mg/g dw or less.

Eight out of twelve (67%) non-transfusion dependent patients with a baseline LIC ≥5 mg/g dw had a decrease of 1 mg/g dw (between at least 1 mg/g dw and up to 4.6 mg/g dry weight) during a 16 week treatment period. Five of the eight patients received iron chelators during this time. Five of the eight patients had a decrease of about 2 mg/g dw during the 16 week treatment period, three of which patients also received iron chelators. Two out of twelve patients had an increase in LIC of ≥1 mg/g dw and one patient had an increase of LIC of 2 mg/g dw during the 16 week treatment period.

In non-transfusion dependent patients, increased hemoglobin was found to correlate with reduction in LIC(R²=0.305, p value=0.063).

10 out of 10 transfusion-dependent patients receiving ActRIIB-hFc (SEQ ID NO:25) treatments at dose levels from 0.6-1.25 mg/kg over 12 weeks experienced a >40% reduction in transfusion burden. 9/10 of these patients experiences a >60% reduction and 2/10 patients experienced a >80% reduction in transfusion burden.

In seven out of seven (100%) transfusion dependent patients with a baseline LIC <7 mg/g dw maintained LIC<7 mg/g dw over a 4-month ActRIIB-hFc (SEQ ID NO:25) treatment period. Five patients experienced a decrease of between about 0.5 mg/g dw to about 2.0 mg/g dw, and two patients experienced an increase of between about 0.5 mg/g dw and about 1.0 mg/g dw during the 4-month ActRIIB-hFc (SEQ ID NO:25) treatment period. All seven patients received iron chelators besides ActRIIB-hFc (SEQ ID NO:25).

Two out of three transfusion dependent patients with a baseline LIC mg/g dw experienced a decrease of 1 mg/g dw (1.96 mg/g dw and 4.7 mg/g dw) over a 16-week ActRIIB-hFc (SEQ ID NO:25) treatment period. All three patients received iron chelators besides ActRIIB-hFc (SEQ ID NO:25).

Three out of three patients with long-term, persistent leg ulcers experienced healing while on treatment with ActRIIB-hFc (SEQ ID NO:25). One non-transfusion dependent patient received ActRIIB-hFc (SEQ ID NO:25) at a dose of 0.4 mg/kg and experienced complete healing after 6 weeks. One transfusion dependent patient received ActRIIB-hFc (SEQ ID NO:25) at a dose of 1.0 mg/kg and experienced complete healing after 18 weeks. One transfusion dependent patient received ActRIIB-hFc (SEQ ID NO:25) at a dose of 1.25 mg/kg and experienced complete healing after 5 weeks.

ActRIIB-hFc (SEQ ID NO:25) was generally well tolerated, with no related serious adverse events reported to date. The most frequent related adverse events included bone pain (23.1% of patients), myalgia (17.9% of patients), headache (15.4% of patients), asthenia (10.3% of patients), pain in extremity (7.7% of patients), influenza (5.1% of patients), Macule (5.1% of patients), and musculoskeletal pain (5.1% of patients).

### 8.4.3 Conclusions

ActRIIB-hFc (SEQ ID NO:25) administered to patients subcutaneously every 3 weeks for up to 16-weeks was generally safe and well-tolerated. A sustained increase in hemoglobin was observed in >50% patients treated with higher doses of ActRIIB-hFc (SEQ ID NO:25; 0.8 to 1.25 mg/kg) in non-transfusion dependent patients. A decreased transfusion burden of >33% was observed in a majority of transfusion dependent patients receiving ActRIIB-hFc (SEQ ID NO:25). A decreased liver iron concentration was observed in a majority of transfusion dependent and non-transfusion dependent patients, with and without iron chelation therapy. Three out of three patients receiving ActRIIB-hFc (SEQ ID NO:25) showed rapid healing of leg ulcers.

### 8.5 Example 5: A Phase 3, Double-Blind, Randomized, Placebo-Controlled, Multicenter Study to Determine the Efficacy and Safety of ACTRIIb-HFC (SEQ ID NO: 25) Versus Placebo in Adults Who Require Regular Red Blood Cell Transfusions Due to Beta Thalassemia

This example is an update to the overview of the phase 3, double blind, randomized, placebo controlled multicenter study to determine the efficacy and safety of ActRIIB-hFc (SEQ ID NO:25) in adults who require regular red blood cell transfusions due to beta-thalassemia described in Example 1 (Section 8.1). The indication for the phase 3 study is adults with transfusion-dependent beta-thalassemia, with a documented diagnosis of beta-thalassemia or hemoglobin E/beta-thalassemia, excluding hemoglobin S/beta-thalassemia.

### 8.5.1 Brief Summary

This is a Phase 3, double-blind, randomized, placebo-controlled, multicenter study to determine the efficacy and safety of ActRIIB-hFc (SEQ ID NO: 25) plus Best supportive care (BSC) versus placebo plus BSC in adults who require regular red blood cell transfusion due to beta-thalassemia.

The study is divided into the Screening/Run-in Period, double-blind Treatment Period, double-blind Long-term Treatment Period, and Post-treatment Follow-up Period.

### 8.5.2 Primary Outcome Measures

A primary outcome measurement of this study is the proportion of subjects with hematological improvement (HI) from Week 13 to Week 24 compared to 12-week prior to randomization, wherein the HI is defined as greater than or equal to 33% reduction from baseline in red blood cell count (RBC) transfusion burden with a reduction of at least 2 units from Week 13 to Week 24 compared to the 12-week; reported as the number of RBC units transfused from Week 13 to Week 24, and in the 12 weeks prior to randomization. The time frame for this measurement is up to approximately week 24.

### 8.5.3 Secondary Outcome Measures

A secondary outcome measurement of this study is the proportion of subjects with hematological improvement (HI) from Week 37 to Week 48 compared to the 12-week interval prior to randomization, wherein the HI is defined as 33% reduction from baseline in red blood cell count (RBC) transfusion burden with a reduction of at least 2 units from Week 37 to Week 48 compared to the 12-week; reported as the number of RBC units transfused from Week 37 to Week 48, and in the 12 weeks prior to randomization. The time frame for this measurement is up to approximately 48 weeks.

Another secondary outcome measurement of this study is the proportion of subjects greater than or equal to 50% reduction from baseline in RBC transfusion burden with a reduction of at least 2 units from Week 37 to Week 48 compared to the 12-week interval prior to randomization for luspatercept plus BSC versus placebo plus BSC, wherein a reduction of greater than or equal to 50% in transfusion burden is defined as a reduction of at least 2 units from week 37 to week 48 compared to the 12 week interval prior to randomization for luspatercept plus (best supportive care) BSC Versus placebo plus BSC; reported as the number of RBC units transfused from Week 37 to Week 48, and in the 12 weeks prior to randomization. The time frame for this measurement is up to approximately 48 weeks.

Another secondary outcome measurement of this study is the proportion of subjects with greater than or equal to 50% reduction from baseline in RBC transfusion burden with a reduction of at least 2 units from Week 13 to Week 24 compared to the 12-week interval prior to randomization for luspatercept plus BSC versus placebo plus BSC, wherein a reduction of greater than or equal to 50% in transfusion burden is defined as a reduction of at least 2 units from week 13 to week 24 compared to the 12 week interval prior to randomization for luspatercept plus (best supportive care) BSC Versus placebo plus BSC; reported as the number of RBC units transfused from Week 37 to Week 48, and in the 12 weeks prior to randomization. The time frame for this measurement is up to approximately 24 weeks.

Another secondary outcome measurement of this study is the mean change from baseline in transfusion burden (RBC units) from Week 13 to Week 24. The time frame for this measurement is up to approximately 24 weeks.

Another secondary outcome measurement of this study is the mean change from baseline in liver iron concentration (LIC, mg/g dry weight) by magnetic resonance imaging (MRI). The time frame for this measurement is up to approximately 24 weeks.

Another secondary outcome measurement of this study is the mean change from baseline in mean daily dose of iron chelation therapy. The time frame of this measurement is up to approximately 48 weeks.

Another secondary outcome measurement of this study is the mean change from baseline in serum ferritin. The time frame of this measurement is up to approximately 48 weeks.

Another secondary outcome measurement of this study is the mean change from baseline in total hip and lumbar spine bone mineral density (BMD) by dual energy x-ray absorptiometry (DXA). The timeframe for this measurement is up to approximately 48 weeks.

Another secondary outcome measurement of this study is the mean change from baseline in myocardial iron by MRI (e.g., T2 MRI). The time frame for this measurement is up to approximately 48 weeks.

Another secondary outcome measurement of this study is the TranQOL Quality of Life tool administered within 4 weeks prior to Dose 1 Day 1, and weeks 12, 24, 36 and 48, then every 12 weeks during long term period. The change from baseline in the scores will be evaluated. The TranQol is a tool specific to this population. The TranQol is a new disease-specific quality of life instrument developed for adult Beta-thalassemia patients. Summary statistics for scores from the pre-specified domains of emotional and the School/Career domains and the total score will be calculated at each administration time point (baseline, weeks 12, 24, 36 and 48 and then every 12 weeks during the Long-term Treatment Period) for the total sample and each treatment group. The time frame for this measurement is up to approximately 3 years.

Another secondary outcome measurement of this study is the Quality of Life tool administered within 4 weeks prior to Dose 1 Day 1, and weeks 12, 24, 36 and 48, then every 12 weeks during long term period. The SF-36 Version 2.0 is a self-administered instrument consisting of 8 multi-item scales that assess 8 health domains: physical functioning, role-physical, bodily pain, general health, vitality, social functioning, role-emotional, and mental health. Two overall summary scores (Physical Component and Mental Component) can also be obtained. The time frame for this measurement is up to approximately 3 years.

Another secondary outcome measurement of this study is the effect of ActRIIB-hFc (SEQ ID NO: 25) on healthcare resource utilization versus placebo. Aggregation of hospitalizations, prior concomitant therapies and surgeries, as well as RBC transfusion utilization will be evaluated. The timeframe for this measurement is up to approximately 3 years.

Another secondary outcome measurement of this study is the proportion of subjects who are transfusion-independent for greater than or equal to 8 weeks during treatment. This can be evaluated by myocardial iron levels determined by MRI (e.g., T2 MRI). The timeframe for this measurement is up to approximately 48 weeks.

Another secondary outcome measurement of this study is the duration of reduction in transfusion burden. The duration of the first response will be calculated for each subject who achieves a response. The time frame for this measurement is up to approximately 48 weeks.

Another secondary outcome measurement of this study is the duration of transfusion independence, for example, the absence of any transfusion during any consecutive rolling 8-week time interval within the treatment period, i.e., days 1 to 56, days 2 to 57, etc. The timeframe for this measurement is up to approximately 48 weeks.

Another secondary outcome measurement of this study is the time to erythroid response. The timeframe for this measurement is up to approximately 48 weeks.

Another secondary outcome measurement of this study is the post-baseline transfusion events frequency versus placebo. The annualized mean change from baseline number of transfusion events will be summarized by treatment groups. The time frame for this measurement is up to approximately 48 weeks.

Another secondary outcome measurement of this study is the pharmacokinetic area under the plasma concentration-time curve. The timeframe for this measurement is up to 9 weeks post the last dose.

Another secondary outcome measurement of this study is the pharmacokinetic maximum observed concentration in plasma. The timeframe for this measurement is up to 9 weeks post the last dose.

### 8.5.4 Safety Outcome Measures

The number of participants with adverse events will be evaluated for up to approximately 3.5 years.

### 8.5.5 Arms/Interventions

Subjects will be administered ActRIIB-hFc (SEQ ID NO: 25) plus best supportive care (BSC). ActRIIB-hFc (SEQ ID NO: 25) will be administered to the subject subcutaneously once every 21 days. Subjects will start with ActRIIB-hFc (SEQ ID NO: 25) at a 1 mg/kg dose level.

Alternatively, subjects will be administered placebo plus best supportive care (BSC). The placebo will be normal saline solution, administered to the subject subcutaneously once every 21 days.

### 8.5.6 Inclusion Criteria

Subjects must satisfy the following criteria to be enrolled in the study: (1) male or female, at least 18 years of age at the time of signing the informed consent document (ICF); (2) subject must understand and voluntarily sign an Inform Consent Form prior to any study-related assessments/procedures being conducted; (3) subject is willing and able to adhere to the study visit schedule and other protocol requirements; (4) documented diagnosis of β-thalassemia or Hemoglobin E/β-thalassemia; (5) regularly transfused, defined as: 6-20 Red Blood Cell (RBC) units in the 24 weeks prior to randomization and no transfusion-free period for 35 days during that period; 1 unit in this protocol refers to a quantity of packed RBCs derived from approximately 400-500 mL of donated blood; (6) performance status: Eastern Cooperative Oncology Group (ECOG) score of 0 or 1; (7) a female of childbearing potential (FCBP) for this study is defined as a female who: (a) has achieved menarche at some point, (b) has not undergone a hysterectomy or bilateral oophorectomy or (c) has not been naturally postmenopausal (amenorrhea following cancer therapy does not rule out childbearing potential) for at least 24 consecutive months (i.e., has had menses at any time in the preceding 24 consecutive months); FCBP participating in the study must: (a) have two negative pregnancy tests as verified by the Investigator prior to starting study therapy; he must agree to ongoing pregnancy testing during the course of the study, and after end of study treatment; this applies even if the subject practices true abstinence from heterosexual contact; and (b) either commit to true abstinence from heterosexual contact (which must be reviewed on a monthly basis and source documented) or agree to use, and be able to comply with, effective contraception without interruption, 28 days prior to starting investigational product, during the study therapy (including dose interruptions), and for 12 weeks (approximately five times the mean terminal half-life of luspatercept based on multiple-dose Pharmacokinetic PK) data) after discontinuation of study therapy; (8) male subjects must practice true abstinence or agree to use a condom during sexual contact with a pregnant female or a female of childbearing potential while participating in the study, during dose interruptions and for at least 12 weeks (approximately five times the mean terminal half-life of luspatercept based on multiple-dose PK data) following investigational product discontinuation, even if he has undergone a successful vasectomy.

### 8.5.7 Exclusion Criteria

The presence of any of the following will exclude a subject from enrollment: (1) any significant medical condition, laboratory abnormality, or psychiatric illness that would prevent the subject from participating in the study; (2) any condition including the presence of laboratory abnormalities, which places the subject at unacceptable risk if he/she were to participate in the study; (3) any condition that confounds the ability to interpret data from the study; (4) a diagnosis of Hemoglobin S/β-thalassemia or alpha (a)-thalassemia (eg, Hemoglobin H); β-thalassemia combined with α-thalassemia is allowed; (5) evidence of active hepatitis C (HCV) infection, or active infectious hepatitis B, or known positive human immunodeficiency virus (HIV); (6) deep vein thrombosis (DVT) or stroke requiring medical intervention 24 weeks prior to randomization; (7) chronic anticoagulant therapy 28 days prior to randomization, Low Molecular Weight (LMW) heparin for Sinus venous Thrombosis (SVT) and chronic aspirin are allowed; (8) platelet count >1000×10⁹/L; (9) insulin-dependent diabetes, ie, chronic treatment with insulin; (10) treatment with another investigational drug or device 28 days prior to randomization; (11) prior exposure to ActRIIA-hFc (SEQ ID NO: 7) or ActRIIB-hFc (SEQ ID NO: 25); (12) use of an erythropoiesis-stimulating agent (ESA)<24 weeks prior to randomization; (13) iron chelation therapy, if initiated 24 weeks prior to randomization (allowed if initiated >24 weeks before or during treatment); (14) hydroxyurea treatment 24 weeks prior to randomization; (15) pregnant or lactating females; (16) uncontrolled hypertension. Controlled hypertension for this protocol is considered Grade 1 according to NCI Common Terminology Criteria for Adverse Events (CTCAE) version 4.0 (current active minor version); (17) major organ damage, including: (a) liver disease with alanine aminotransferase (ALT) >3×the upper limit of normal (ULN) or histopathological evidence of liver cirrhosis/fibrosis on liver biopsy; (b) heart disease, heart failure as classified by the New York Heart Association (NYHA) classification 3 or higher, or significant arrhythmia requiring treatment, or recent myocardial infarction within 6 months of randomization; (c) lung disease, including pulmonary fibrosis or pulmonary hypertension which are clinically significant; and/or (d) creatinine clearance <60 mL/min (per Cockroff-Gault method); (18) proteinuria ≥Grade 3 according to NCI CTCAE version 4.0 (current active minor version); (19) adrenal insufficiency; (20) major surgery 12 weeks prior to randomization (subjects must have completely recovered from any previous surgery prior to randomization); (21) history of severe allergic or anaphylactic reactions or hypersensitivity to recombinant proteins or excipients in the investigational product (see Investigator Brochure); (22) cytotoxic agents, immunosuppressants 28 days prior to randomization.

### 9. DESCRIPTION OF THE SEQUENCES

TABLE 3
Sequence Information

| SEQ | | |
|---|---|---|
| ID | | |
| NO: | Description | Sequence |
| 1 | human ActRIIA precursor | MGAAAKLAFAVFLISCSSGAILGRSETQECLFFNA |
| | polypeptide | NWEKDRTNQTGVEPCYGDKDKRRHCFATWKNIS |
| | | GSIEIVKQGCWLDDINCYDRTDCVEKKDSPEVYF |
| | | CCCEGNMCNEKFSYFPEMEVTQPTSNPVTPKPPY |
| | | YNILLYSLVPLMLIAGIVICAFWVYREIHKMAYPP |
| | | VLVPTQDPGPPPPSPLLGLKPLQLLEVKARGRFGC |
| | | VWKAQLLNEYVAVKIFPIQDKQSWQNEYEVYSLP |
| | | GMKHENILQFIGAEKRGTSVDVDLWLITAFHEKG |
| | | SLSDFLKANVVSWNELCHIAETMARGLAYLHEDI |
| | | PGLKDGHKPAISHRDIKSKNVLLKNNLTACIADFG |
| | | LALKFEAGKSAGDTHGQVGTRRYMAPEVLEGAI |
| | | NFQRDAFLRIDMYAMGLVLWELASRCTAADGPV |
| | | DEYMLPFEEEIGQHPSLEDMQEVVVHKKKRPVLR |
| | | DYWQKHAGMAMLCETIEECWDHDAEARLSAGC |
| | | VGERITQMQRLTNIITTEDIVTVVTMVTNVDFPPK |
| | | ESSL |
| 2 | human ActRIIA soluble | ILGRSETQECLFFNANWEKDRTNQTGVEPCYGDK |
| | (extracellular), processed | DKRRHCFATWKNISGSIEIVKQGCWLDDINCYDR |
| | polypeptide sequence | TDCVEKKDSPEVYFCCCEGNMCNEKFSYFPEMEV |
| | | TQPTSNPVTPKPP |
| 3 | human ActRIIA soluble | ILGRSETQECLFFNANWEKDRTNQTGVEPCYGDK |
| | (extracellular), processed | DKRRHCFATWKNISGSIEIVKQGCWLDDINCYDR |
| | polypeptide sequence with | TDCVEKKDSPEVYFCCCEGNMCNEKFSYFPEM |
| | the C-terminal 15 amino | |
| | acids deleted | |
| 4 | nucleic acid sequence | ATGGGAGCTGCTGCAAAGTTGGCGTTTGCCGTC |
| | encoding human ActRIIA | TTTCTTATCTCCTGTTCTTCAGGTGCTATACTTG |
| | precursor protein | GTAGATCAGAAACTCAGGAGTGTCTTTTCTTTA |
| | | ATGCTAATTGGGAAAAAGACAGAACCAATCAA |
| | | ACTGGTGTTGAACCGTGTTATGGTGACAAAGAT |
| | | AAACGGCGGCATTGTTTTGCTACCTGGAAGAAT |
| | | ATTTCTGGTTCCATTGAAATAGTGAAACAAGGT |
| | | TGTTGGCTGGATGATATCAACTGCTATGACAGG |
| | | ACTGATTGTGTAGAAAAAAAAGACAGCCCTGA |
| | | AGTATATTTTTGTTGCTGTGAGGGCAATATGTG |
| | | TAATGAAAAGTTTTCTTATTTTCCAGAGATGGA |
| | | AGTCACACAGCCCACTTCAAATCCAGTTACACC |
| | | TAAGCCACCCTATTACAACATCCTGCTCTATTCC |
| | | TTGGTGCCACTTATGTTAATTGCGGGGATTGTC |
| | | ATTTGTGCATTTTGGGTGTACAGGCATCACAAG |
| | | ATGGCCTACCCTCCTGTACTTGTTCCAACTCAA |
| | | GACCCAGGACCACCCCCACCTTCTCCATTACTA |
| | | GGGTTGAAACCACTGCAGTTATTAGAAGTGAAA |
| | | GCAAGGGGAAGATTTGGTTGTGTCTGGAAAGCC |
| | | CAGTTGCTTAACGAATATGTGGCTGTCAAAATA |
| | | TTTCCAATACAGGACAAACAGTCATGGCAAAAT |
| | | GAATACGAAGTCTACAGTTTGCCTGGAATGAAG |
| | | CATGAGAACATATTACAGTTCATTGGTGCAGAA |
| | | AAACGAGGCACCAGTGTTGATGTGGATCTTTGG |
| | | CTGATCACAGCATTTCATGAAAAGGGTTCACTA |
| | | TCAGACTTTCTTAAGGCTAATGTGGTCTCTTGG |
| | | AATGAACTGTGTCATATTGCAGAAACCATGGCT |
| | | AGAGGATTGGCATATTTACATGAGGATATACCT |
| | | GGCCTAAAAGATGGCCACAAACCTGCCATATCT |
| | | CACAGGGACATCAAAAGTAAAAATGTGCTGTT |
| | | GAAAAACAACCTGACAGCTTGCATTGCTGACTT |
| | | TGGGTTGGCCTTAAAATTTGAGGCTGGCAAGTC |
| | | TGCAGGCGATACCCATGGACAGGTTGGTACCCG |
| | | GAGGTACATGGCTCCAGAGGTATTAGAGGGTG |
| | | CTATAAACTTCGAAAGGGATGCATTTTTGAGGA |
| | | TAGATATGTATGCCATGGGATTAGTCCTATGGG |
| | | AACTGGCTTCTCGCTGTACTGCTGCAGATGGAC |
| | | CTGTAGATGAATACATGTTGCCATTTGAGGAGG |
| | | AAATTGGCCAGCATCCATCTCTTGAAGACATGC |
| | | AGGAAGTTGTTGTGCATAAAAAAAAGAGGCCT |
| | | GTTTTAAGAGATTATTGGCAGAAACATGCTGGA |
| | | ATGGCAATGCTCTGTGAAACCATTGAAGAATGT |
| | | TGGGATCACGACGCAGAAGCCAGGTTATCAGCT |
| | | GGATGTGTAGGTGAAAGAATTACCCAGATGCA |
| | | GAGACTAACAAATATTATTACCACAGAGGACAT |
| | | TGTAACAGTGGTCACAATGGTGACAAATGTTGA |
| | | CTTTCCTCCCAAAGAATCTAGTCTATGA |
| 5 | nucleic acid sequence | ATACTTGGTAGATCAGAAACTCAGGAGTGTCTT |
| | encoding a human ActRIIA | TTCTTTAATGCTAATTGGGAAAAAGACAGAACC |
| | soluble (extracellular) | AATCAAACTGGTGTTGAACCGTGTTATGGTGAC |
| | polypeptide | AAAGATAAACGGCGGCATTGTTTTGCTACCTGG |
| | | AAGAATATTTCTGGTTCCATTGAAATAGTGAAA |
| | | CAAGGTTGTTGGCTGGATGATATCAACTGCTAT |
| | | GACAGGACTGATTGTGTAGAAAAAAAAGACAG |
| | | CCCTGAAGTATATTTTTGTTGCTGTGAGGGCAA |
| | | TATGTGTAATGAAAAGTTTTCTTATTTTCCAGAG |
| | | ATGGAAGTCACACAGCCCACTTCAAATCCAGTT |
| | | ACACCTAAGCCACCC |
| 6 | fusion protein comprising a | THTCPPCPAPELLGGPSVFLFPPKPKDTLMISRTPE |
| | soluble extracellular | VTCVVVDX1VSHEDPEVKFNWYVDGVEVHNAKT |
| | domain of ActRIIA fused to | KPREEQYNSTYRVVSVLTVLHQDWLNGKEYKCK |
| | an Fc domain | X2VSNKALPVPIEKTISKAKGQPREPQVYTLPPSRE |
| | | EMTKNQVSLTCLVKGFYPSDIAVEWESNGQPENN |
| | | YKTTPPVLDSDGPFFLYSKLTVDKSRWQQGNVFS |
| | | CSVMHEALHNX3HYTQKSLSLSPGK*(whereinX1 |
| | | isDorA;X2isKorAandX3isNorA) |
| 7 | Extracellular domain of | ILGRSETQECLFFNANWEKDRTNQTGVEPCYGDK |
| | human ActRIIA fused to a | DKRRHCFATWKNISGSIEIVKQGCWLDDINCYDR |
| | human Fc domain | TDCVEKKDSPEVYFCCCEGNMCNEKFSYFPEMEV |
| | | TQPTSNPVTPKPPTGGGTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPVPIEKTISKAKGQ |
| | | PREPQVYTLPPSREEMTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGK |
| 8 | Leader sequence of Honey | MKFLVNVALVFMVVYISYIYA |
| | bee mellitin (HBML) | |
| 9 | Leader sequence of Tissue | MDAMKRGLCCVLLLCGAVFVSP |
| | Plasminogen Activator | |
| | (TPA) | |
| 10 | Native ActRIIA leader | MGAAAKLAFAVFLISCSSGA |
| 11 | ActRIIA-hFc and | ILGRSETQE |
| | mActRIIA-Fc N-terminal | |
| | sequence | |
| 12 | ActRIIA-Fc Protein with | ILGRSETQECLFFNANWEKDRTNQTGVEPCYGDK |
| | deletion of the C-terminal | DKRRHCFATWKNISGSIEIVKQGCWLDDINCYDR |
| | 15 amino acids of the | TDCVEKKDSPEVYFCCCEGNMCNEKFSYFPEMTG |
| | extracellular domain of | GGTHTCPPCPAPELLGGPSVFLFPPKPKDTLMISRT |
| | ActRIIA | PEVTCVVVDVSHEDPEVKFNWYVDGVEVHNAKT |
| | | KPREEQYNSTYRVVSVLTVLHQDWLNGKEYKCK |
| | | VSNKALPVPIEKTISKAKGQPREPQVYTLPPSREE |
| | | MTKNQVSLTCLVKGFYPSDIAVEWESNGQPENNY |
| | | KTTPPVLDSDGSFFLYSKLTVDKSRWQQGNVFSC |
| | | SVMHEALHNHYTQKSLSLSPGK |
| 13 | Unprocessed ActRIIA-hFc | MDAMKRGLCCVLLLCGAVFVSPGAAILGRSETQE |
| | with TPA leader sequence | CLFFNANWEKDRTNQTGVEPCYGDKDKRRHCFA |
| | | TWKNISGSIEIVKQGCWLDDINCYDRTDCVEKKD |
| | | SPEVYFCCCEGNMCNEKFSYFPEMEVTQPTSNPV |
| | | TPKPPTGGGTHTCPPCPAPELLGGPSVFLFPPKPKD |
| | | TLMISRTPEVTCVVVDVSHEDPEVKFNWYVDGVE |
| | | VHNAKTKPREEQYNSTYRVVSVLTVLHQDWLNG |
| | | KEYKCKVSNKALPVPIEKTISKAKGQPREPQVYTL |
| | | PPSREEMTKNQVSLTCLVKGFYPSDIAVEWESNG |
| | | QPENNYKTTPPVLDSDGSFFLYSKLTVDKSRWQQ |
| | | GNVFSCSVMHEALHNHYTQKSLSLSPGK |
| 14 | Nucleic acid sequence | ATGGATGCAATGAAGAGAGGGCTCTGCTGTGTG |
| | encoding Unprocessed | CTGCTGCTGTGTGGAGCAGTCTTCGTTTCGCCC |
| | ActRIIA-hFc with TPA | GGCGCCGCTATACTTGGTAGATCAGAAACTCAG |
| | leader sequence | GAGTGTCTTTTTTTAATGCTAATTGGGAAAAAG |
| | | ACAGAACCAATCAAACTGGTGTTGAACCGTGTT |
| | | ATGGTGACAAAGATAAACGGCGGCATTGTTTTG |
| | | CTACCTGGAAGAATATTTCTGGTTCCATTGAAT |
| | | AGTGAAACAAGGTTGTTGGCTGGATGATATCAA |
| | | CTGCTATGACAGGACTGATTGTGTAGAAAAAAA |
| | | AGACAGCCCTGAAGTATATTTCTGTTGCTGTGA |
| | | GGGCAATATGTGTAATGAAAAGTTTTCTTATTT |
| | | TCCGGAGATGGAAGTCACACAGCCCACTTCAAA |
| | | TCCAGTTACACCTAAGCCACCCACCGGTGGTGG |
| | | AACTCACACATGCCCACCGTGCCCAGCACCTGA |
| | | ACTCCTGGGGGGACCGTCAGTCTTCCTCTTCCC |
| | | CCCAAAACCCAAGGACACCCTCATGATCTCCCG |
| | | GACCCCTGAGGTCACATGCGTGGTGGTGGACGT |
| | | GAGCCACGAAGACCCTGAGGTCAAGTTCAACT |
| | | GGTACGTGGACGGCGTGGAGGTGCATAATGCC |
| | | AAGACAAAGCCGCGGGAGGAGCAGTACAACAG |
| | | CACGTACCGTGTGGTCAGCGTCCTCACCGTCCT |
| | | GCACCAGGACTGGCTGAATGGCAAGGAGTACA |
| | | AGTGCAAGGTCTCCAACAAAGCCCTCCCAGTCC |
| | | CCATCGAGAAAACCATCTCCAAAGCCAAAGGG |
| | | CAGCCCCGAGAACCACAGGTGTACACCCTGCCC |
| | | CCATCCCGGGAGGAGATGACCAAGAACCAGGT |
| | | CAGCCTGACCTGCCTGGTCAAAGGCTTCTATCC |
| | | CAGCGACATCGCCGTGGAGTGGGAGAGCAATG |
| | | GGCAGCCGGAGAACAACTACAAGACCACGCCT |
| | | CCCGTGCTGGACTCCGACGGCTCCTTCTTCCTCT |
| | | ATAGCAAGCTCACCGTGGACAAGAGCAGGTGG |
| | | CAGCAGGGGAACGTCTTCTCATGCTCCGTGATG |
| | | CATGAGGCTCTGCACAACCACTACACGCAGAA |
| | | GAGCCTCTCCCTGTCTCCGGTAAATGAGAATTC |
| 15 | human ActRIIB soluble | ETRECIYYNANWELERTNQSGLERCEGEQDKRLH |
| | (extracellular), processed | CYASWRNSSGTIELVKKGCWDDDFNCYDRQECV |
| | polypeptide sequence with | ATEENPQVYFCCCEGNFCNERFTHLPEAGGP |
| | the N-terminal 6 amino | EVTYEPPP |
| | acids of the EC domain | |
| | deleted and the C-terminal | |
| | 4 amino acids of the EC | |
| | domain deleted (amino | |
| | acids 25-130 of SEQ ID | |
| | NO: 28) and with an L79D | |
| | mutation | |
| 16 | human ActRIIB precursor | MTAPWVALALLWGSLWPGSGRGEAETRECIYYN |
| | protein sequence (A64) | ANWELERTNQSGLERCEGEQDKRLHCYASWANS |
| | | SGTIELVKKGCWLDDFNCYDRQECVATEENPQV |
| | | YFCCCEGNFCNERFTHLPEAGGPEVTYEPPPTAPT |
| | | LLTVLAYSLLPIGGLSLIVLLAFWMYRHRKPPYGH |
| | | VDIHEDPGPPPPSPLVGLKPLQLLEIKARGRFGCV |
| | | WKAQLMNDFVAVKIFPLQDKQSWQSEREIFSTPG |
| | | MKHENLLQFIAAEKRGSNLEVELWLITAFHDKGS |
| | | LTDYLKGNIITWNELCHVAETMSRGLSYLHEDVP |
| | | WCRGEGHKPSIAHRDFKSKNVLLKSDLTAVLADF |
| | | GLAVRFEPGKPPGDTHGQVGTRRYMAPEVLEGAI |
| | | NFQRDAFLRIDMYAMGLVLWELVSRCKAADGPV |
| | | DEYMLPFEEEIGQHPSLEELQEVVVHKKMRPTIKD |
| | | HWLKHPGLAQLCVTIEECWDHDAEARLSAGCVE |
| | | ERVSLIRRSVNGTTSDCLVSLVTSVTNVDLPPKES |
| | | SI |
| 17 | human ActRIIB soluble | SGRGEAETRECIYYNANWELERTNQSGLERCEGE |
| | (extracellular), processed | QDKRLHCYASWANSSGTIELVKKGCWLDDFNCY |
| | polypeptide sequence | DRQECVATEENPQVYFCCCEGNFCNERFTHLPEA |
| | (amino acids 19-134 of | GGPEVTYEPPPTAPT |
| | SEQ ID NO: 16) | |
| 18 | human ActRIIB soluble | SGRGEAETRECIYYNANWELERTNQSGLERCEGE |
| | (extracellular), processed | QDKRLHCYASWANSSGTIELVKKGCWLDDFNCY |
| | polypeptide sequence with | DRQECVATEENPQVYFCCCEGNFCNERFTHLPEA |
| | the C-terminal 15 amino | |
| | acids deleted (amino acids | |
| | 19-119 of SEQ ID NO: 16) | |
| 19 | nucleic acid sequence | ATGACGGCGCCCTGGGTGGCCCTCGCCCTCCTC |
| | encoding a human ActRIIB | TGGGGATCGCTGTGGCCCGGCTCTGGGCGTGGG |
| | (A64) precursor protein | GAGGCTGAGACACGGGAGTGCATCTACTACAA |
| | | CGCCAACTGGGAGCTGGAGCGCACCAACCAGA |
| | | GCGGCCTGGAGCGCTGCGAAGGCGAGCAGGAC |
| | | AAGCGGCTGCACTGCTACGCCTCCTGGGCCAAC |
| | | AGCTCTGGCACCATCGAGCTCGTGAAGAAGGG |
| | | CTGCTGGCTAGATGACTTCAACTGCTACGATAG |
| | | GCAGGAGTGTGTGGCCACTGAGGAGAACCCCC |
| | | AGGTGTACTTCTGCTGCTGTGAAGGCAACTTCT |
| | | GCAACGAGCGCTTCACTCATTTGCCAGAGGCTG |
| | | GGGGCCCGGAAGTCACGTACGAGCCACCCCCG |
| | | ACAGCCCCCACCCTGCTCACGGTGCTGGCCTAC |
| | | TCACTGCTGCCCATCGGGGGCCTTTCCCTCATC |
| | | GTCCTGCTGGCCTTTTGGATGTACCGGCATCGC |
| | | AAGCCCCCCTACGGTCATGTGGACATCCATGAG |
| | | GACCCTGGGCCTCCACCACCATCCCCTCTGGTG |
| | | GGCCTGAAGCCACTGCAGCTGCTGGAGATCAA |
| | | GGCTCGGGGGCGCTTTGGCTGTGTCTGGAAGGC |
| | | CCAGCTCATGAATGACTTTGTAGCTGTCAAGAT |
| | | CTTCCCACTCCAGGACAAGCAGTCGTGGCAGAG |
| | | TGAACGGGAGATCTTCAGCACACCTGGCATGAA |
| | | GCACGAGAACCTGCTACAGTTCATTGCTGCCGA |
| | | GAAGCGAGGCTCCAACCTCGAAGTAGAGCTGT |
| | | GGCTCATCACGGCCTTCCATGACAAGGGCTCCC |
| | | TCACGGATTACCTCAAGGGGAACATCATCACAT |
| | | GGAACGAACTGTGTCATGTAGCAGAGACGATG |
| | | TCACGAGGCCTCTCATACCTGCATGAGGATGTG |
| | | CCCTGGTGCCGTGGCGAGGGCCACAAGCCGTCT |
| | | ATTGCCCACAGGGACTTTAAAAGTAAGAATGTA |
| | | TTGCTGAAGAGCGACCTCACAGCCGTGCTGGCT |
| | | GACTTTGGCTTGGCTGTTCGATTTGAGCCAGGG |
| | | AAACCTCCAGGGGACACCCACGGACAGGTAGG |
| | | CACGAGACGGTACATGGCTCCTGAGGTGCTCGA |
| | | GGGAGCCATCAACTTCCAGAGAGATGCCTTCCT |
| | | GCGCATTGACATGTATGCCATGGGGTTGGTGCT |
| | | GTGGGAGCTTGTGTCTCGCTGCAAGGCTGCAGA |
| | | CGGACCCGTGGATGAGTACATGCTGCCCTTTGA |
| | | GGAAGAGATTGGCCAGCACCCTTCGTTGGAGG |
| | | AGCTGCAGGAGGTGGTGGTGCACAAGAAGATG |
| | | AGGCCCACCATTAAAGATCACTGGTTGAAACAC |
| | | CCGGGCCTGGCCCAGCTTTGTGTGACCATCGAG |
| | | GAGTGCTGGGACCATGATGCAGAGGCTCGCTTG |
| | | TCCGCGGGCTGTGTGGAGGAGCGGGTGTCCCTG |
| | | ATTCGGAGGTCGGTCAACGGCACTACCTCGGAC |
| | | TGTCTCGTTTCCCTGGTGACCTCTGTCACCAATG |
| | | TGGACCTGCCCCCTAAAGAGTCAAGCATCTAA |
| 20 | fusion protein comprising a | SGRGEAETRECIYYNANWELERTNQSGLERCEGE |
| | soluble extracellular | QDKRLHCYASWANSSGTIELVKKGCWLDDFNCY |
| | domain of ActRIIB (A64; | DRQECVATEENPQVYFCCCEGNFCNERFTHLPEA |
| | SEQ ID NO: 17) fused to an | GGPEVTYEPPPTAPTGGGTHTCPPCPAPELLGGPS |
| | Fc domain | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPVPIEKTISKAKG |
| | | QPREPQVYTLPPSREEMTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSL |
| | | SPGK |
| 21 | fusion protein comprising a | SGRGEAETRECIYYNANWELERTNQSGLERCEGE |
| | soluble extracellular | QDKRLHCYASWANSSGTIELVKKGCWLDDFNCY |
| | domain of ActRIIB (A64) | DRQECVATEENPQVYFCCCEGNFCNERFTHLPEA |
| | with the C-terminal 15 | GGGTHTCPPCPAPELLGGPSVFLFPPKPKDTLMIS |
| | amino acids deleted (SEQ | RTPEVTCVVVDVSHEDPEVKFNWYVDGVEVHNA |
| | ID NO: 18) fused to an Fc | KTKPREEQYNSTYRVVSVLTVLHQDWLNGKEYK |
| | domain | CKVSNKALPVPIEKTISKAKGQPREPQVYTLPPSR |
| | | EEMTKNQVSLTCLVKGFYPSDIAVEWESNGQPEN |
| | | NYKTTPPVLDSDGSFFLYSKLTVDKSRWQQGNVF |
| | | SCSVMHEALHNHYTQKSLSLSPGK |
| 22 | human ActRIIB soluble | ETRECIYYNANWELERTNQSGLERCEGEQDKRLH |
| | (extracellular), processed | CYASWRNSSGTIELVKKGCWDDDFNCYDRQECV |
| | polypeptide sequence with | ATEENPQVYFCCCEGNFCNERFTHLPEAGGPEVT |
| | the N-terminal 6 amino | YEPP |
| | acids of the EC domain | |
| | deleted and the C-terminal | |
| | 5 amino acids of the EC | |
| | domain deleted (amino | |
| | acids 25-129 of SEQ ID | |
| | NO: 28) and with an L79D | |
| | mutation | |
| 23 | human ActRIIB soluble | ETRECIYYNANWELERTNQSGLERCEGEQDKRLH |
| | (extracellular), processed | CYASWRNSSGTIELVKKGCWDDDFNCYDRQECV |
| | polypeptide sequence with | ATEENPQVYFCCCEGNFCNERFTHLPEAGGPEVT |
| | the N-terminal 6 amino | YEPPPT |
| | acids of the EC domain | |
| | deleted and the C-terminal | |
| | 3 amino acids of the EC | |
| | domain deleted (amino | |
| | acids 25-131 of SEQ ID | |
| | NO: 28) and with an L79D | |
| | mutation | |
| 24 | Unprocessed ActRIIB-Fc | MDAMKRGLCCVLLLCGAVFVSPGAAETRECIYY |
| | fusion protein with the N- | NANWELERTNQSGLERCEGEQDKRLHCYASWRN |
| | terminal 6 amino acids of | SSGTIELVKKGCWDDDFNCYDRQECVATEENPQV |
| | the EC domain deleted and | YFCCCEGNFCNERFTHLPEAGGPEVTYEPPPTGGG |
| | the C-terminal 3 amino | THTCPPCPAPELLGGPSVFLFPPKPKDTLMISRTPE |
| | acids of the EC domain | VTCVVVDVSHEDPEVKFNWYVDGVEVHNAKTK |
| | deleted (amino acids 25-131 | PREEQYNSTYRVVSVLTVLHQDWLNGKEYKCKV |
| | of SEQ ID NO: 28) and with | SNKALPAPIEKTISKAKGQPREPQVYTLPPSREEM |
| | an L79D mutation and with | TKNQVSLTCLVKGFYPSDIAVEWESNGQPENNYK |
| | TPA leader sequence | TTPPVLDSDGSFFLYSKLTVDKSRWQQGNVFSCS |
| | | VMHEALHNHYTQKSLSLSPGK* |
| 25 | Processed ActRIIB-Fc | ETRECIYYNANWELERTNQSGLERCEGEQDKRLH |
| | fusion protein with the N- | CYASWRNSSGTIELVKKGCWDDDFNCYDRQECV |
| | terminal 6 amino acids of | ATEENPQVYFCCCEGNFCNERFTHLPEAGGPEVT |
| | the EC domain deleted and | YEPPPTGGGTHTCPPCPAPELLGGPSVFLFPPKPKD |
| | the C-terminal 3 amino | TLMISRTPEVTCVVVDVSHEDPEVKFNWYVDGVE |
| | acids of the EC domain | VHNAKTKPREEQYNSTYRVVSVLTVLHQDWLNG |
| | deleted (amino acids 25-131 | KEYKCKVSNKALPAPIEKTISKAKGQPREPQVYTL |
| | of SEQ ID NO: 28) and with | PPSREEMTKNQVSLTCLVKGFYPSDIAVEWESNG |
| | an L79D mutation | QPENNYKTTPPVLDSDGSFFLYSKLTVDKSRWQQ |
| | | GNVFSCSVMHEALHNHYTQKSLSLSPGK* |
| 26 | human ActRIIB soluble | GRGEAETRECIYYNANWELERTNQSGLERCEGEQ |
| | (extracellular), processed | DKRLHCYASWANSSGTIELVKKGCWLDDFNCYD |
| | polypeptide sequence | RQECVATEENPQVYFCCCEGNFCNERFTHLPEAG |
| | (amino acids 20-134 of | GPEVTYEPPPTAPT |
| | SEQ ID NO: 16) | |
| 27 | human ActRIIB soluble | GRGEAETRECIYYNANWELERTNQSGLERCEGEQ |
| | (extracellular), processed | DKRLHCYASWANSSGTIELVKKGCWLDDFNCYD |
| | polypeptide sequence with | RQECVATEENPQVYFCCCEGNFCNERFTHLPEA |
| | the C-terminal 15 amino | |
| | acids deleted (amino acids | |
| | 20-119 of SEQ ID NO: 16) | |
| 28 | human ActRIIB precursor | MTAPWVALALLWGSLWPGSGRGEAETRECIYYN |
| | protein sequence (R64) | ANWELERTNQSGLERCEGEQDKRLHCYASWRNS |
| | | SGTIELVKKGCWLDDFNCYDRQECVATEENPQV |
| | | YFCCCEGNFCNERFTHLPEAGGPEVTYEPPPTAPT |
| | | LLTVLAYSLLPIGGLSLIVLLAFWMYRHRKPPYGH |
| | | VDIHEDPGPPPPSPLVGLKPLQLLEIKARGRFGCV |
| | | WKAQLMNDFVAVKIFPLQDKQSWQSEREIFSTPG |
| | | MKHENLLQFIAAEKRGSNLEVELWLITAFHDKGS |
| | | LTDYLKGNIITWNELCHVAETMSRGLSYLHEDVP |
| | | WCRGEGHKPSIAHRDFKSKNVLLKSDLTAVLADF |
| | | GLAVRFEPGKPPGDTHGQVGTRRYMAPEVLEGAI |
| | | NFQRDAFLRIDMYAMGLVLWELVSRCKAADGPV |
| | | DEYMLPFEEEIGQHPSLEELQEVVVHKKMRPTIKD |
| | | HWLKHPGLAQLCVTIEECWDHDAEARLSAGCVE |
| | | ERVSLIRRSVNGTTSDCLVSLVTSVTNVDLPPKES |
| | | SI |
| 29 | human ActRIIB soluble | SGRGEAETRECIYYNANWELERTNQSGLERCEGE |
| | (extracellular), processed | QDKRLHCYASWRNSSGTIELVKKGCWLDDFNCY |
| | polypeptide sequence | DRQECVATEENPQVYFCCCEGNFCNERFTHLPEA |
| | (amino acids 19-134 of | GGPEVTYEPPPTAPT |
| | SEQ ID NO: 28) | |
| 30 | human ActRIIB soluble | SGRGEAETRECIYYNANWELERTNQSGLERCEGE |
| | (extracellular), processed | QDKRLHCYASWRNSSGTIELVKKGCWLDDFNCY |
| | polypeptide sequence with | DRQECVATEENPQVYFCCCEGNFCNERFTHLPEA |
| | the C-terminal 15 amino | |
| | acids deleted (amino acids | |
| | 19-119 of SEQ ID NO: 28) | |
| 31 | human ActRIIB soluble | GRGEAETRECIYYNANWELERTNQSGLERCEGEQ |
| | (extracellular), processed | DKRLHCYASWRNSSGTIELVKKGCWLDDFNCYD |
| | polypeptide sequence | RQECVATEENPQVYFCCCEGNFCNERFTHLPEAG |
| | (amino acids 20-134 of | GPEVTYEPPPTAPT |
| | SEQ ID NO: 28) | |
| 32 | human ActRIIB soluble | GRGEAETRECIYYNANWELERTNQSGLERCEGEQ |
| | (extracellular), processed | DKRLHCYASWRNSSGTIELVKKGCWLDDFNCYD |
| | polypeptide sequence with | RQECVATEENPQVYFCCCEGNFCNERFTHLPEA |
| | the C-terminal 15 amino | |
| | acids deleted (amino acids | |
| | 20-119 of SEQ ID NO: 28) | |
| 33 | human ActRIIB soluble | ETRECIYYNANWELERTNQSGLERCEGEQDKRLH |
| | (extracellular), processed | CYASWANSSGTIELVKKGCWDDDFNCYDRQECV |
| | polypeptide sequence with | ATEENPQVYFCCCEGNFCNERFTHLPEAGGPEVT |
| | the N-terminal 6 amino | YEPPPT |
| | acids of the EC domain | |
| | deleted and the C-terminal | |
| | 3 amino acids of the EC | |
| | domain deleted (amino | |
| | acids 25-131 of SEQ ID | |
| | NO: 16) and with an L79D | |
| | mutation | |
| 34 | Unprocessed ActRIIB-Fc | MDAMKRGLCCVLLLCGAVFVSPGAAETRECIYY |
| | fusion protein with the N- | NANWELERTNQSGLERCEGEQDKRLHCYASWAN |
| | terminal 6 amino acids of | SSGTIELVKKGCWDDDFNCYDRQECVATEENPQV |
| | the EC domain deleted and | YFCCCEGNFCNERFTHLPEAGGPEVTYEPPPTGGG |
| | the C-terminal 3 amino | THTCPPCPAPELLGGPSVFLFPPKPKDTLMISRTPE |
| | acids of the EC domain | VTCVVVDVSHEDPEVKFNWYVDGVEVHNAKTK |
| | deleted (amino acids 25-131 | PREEQYNSTYRVVSVLTVLHQDWLNGKEYKCKV |
| | of SEQ ID NO: 16) and with | SNKALPAPIEKTISKAKGQPREPQVYTLPPSREEM |
| | an L79D mutation and with | TKNQVSLTCLVKGFYPSDIAVEWESNGQPENNYK |
| | TPA leader sequence | TTPPVLDSDGSFFLYSKLTVDKSRWQQGNVFSCS |
| | | VMHEALHNHYTQKSLSLSPGK* |
| 35 | Processed ActRIIB-Fc | ETRECIYYNANWELERTNQSGLERCEGEQDKRLH |
| | fusion protein with the N- | CYASWANSSGTIELVKKGCWDDDFNCYDRQECV |
| | terminal 6 amino acids of | ATEENPQVYFCCCEGNFCNERFTHLPEAGGPEVT |
| | the EC domain deleted and | YEPPPTGGGTHTCPPCPAPELLGGPSVFLFPPKPKD |
| | the C-terminal 3 amino | TLMISRTPEVTCVVVDVSHEDPEVKFNWYVDGVE |
| | acids of the EC domain | VHNAKTKPREEQYNSTYRVVSVLTVLHQDWLNG |
| | deleted (amino acids 25-131 | KEYKCKVSNKALPAPIEKTISKAKGQPREPQVYTL |
| | of SEQ ID NO: 16) and with | PPSREEMTKNQVSLTCLVKGFYPSDIAVEWESNG |
| | an L79D mutation | QPENNYKTTPPVLDSDGSFFLYSKLTVDKSRWQQ |
| | | GNVFSCSVMHEALHNHYTQKSLSLSPGK* |
| 36 | human ActRIIB soluble | GRGEAETRECIYYNANWELERTNQSGLERCEGEQ |
| | (extracellular), processed | DKRLHCYASWRNSSGTIELVKKGCWDDDFNCYD |
| | polypeptide sequence | RQECVATEENPQVYFCCCEGNFCNERFTHLPEAG |
| | (amino acids 20-134 of | GPEVTYEPPPTAPT |
| | SEQ ID NO: 28) with L79D | |
| | mutation | |
| 37 | human ActRIIB soluble | GRGEAETRECIYYNANWELERTNQSGLERCEGEQ |
| | (extracellular), processed | DKRLHCYASWANSSGTIELVKKGCWDDDFNCYD |
| | polypeptide sequence | RQECVATEENPQVYFCCCEGNFCNERFTHLPEAG |
| | (amino acids 20-134 of | GPEVTYEPPPTAPT |
| | SEQ ID NO: 16) with L79D | |
| | mutation | |
| 38 | human ActRIIB soluble | GRGEAETRECIYYNANWELERTNQSGLERCEGEQ |
| | (extracellular), processed | DKRLHCYASWRNSSGTIELVKKGCWDDDFNCYD |
| | polypeptide sequence | RQECVATEENPQVYFCCCEGNFCNERFTHLPEAG |
| | (amino acids 20-134 of | GPEVTYEPPPTAPTGGGTHTCPPCPAPELLGGPSV |
| | SEQ ID NO: 28) with L79D | FLFPPKPKDTLMISRTPEVTCVVVDVSHEDPEVKF |
| | mutation fused to an Fc | NWYVDGVEVHNAKTKPREEQYNSTYRVVSVLTV |
| | domain with a GGG linker | LHQDWLNGKEYKCKVSNKALPAPIEKTISKAKGQ |
| | | PREPQVYTLPPSREEMTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGK* |
| 39 | human ActRIIB soluble | GRGEAETRECIYYNANWELERTNQSGLERCEGEQ |
| | (extracellular), processed | DKRLHCYASWANSSGTIELVKKGCWDDDFNCYD |
| | polypeptide sequence | RQECVATEENPQVYFCCCEGNFCNERFTHLPEAG |
| | (amino acids 20-134 of | GPEVTYEPPPTAPTGGGTHTCPPCPAPELLGGPSV |
| | SEQ ID NO: 16) with L79D | FLFPPKPKDTLMISRTPEVTCVVVDVSHEDPEVKF |
| | mutation fused to an Fc | NWYVDGVEVHNAKTKPREEQYNSTYRVVSVLTV |
| | domain | LHQDWLNGKEYKCKVSNKALPAPIEKTISKAKGQ |
| | | PREPQVYTLPPSREEMTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGK* |
| 40 | human ActRIIB soluble | MDAMKRGLCCVLLLCGAVFVSPGASGRGEAETR |
| | (extracellular), processed | ECIYYNANWELERTNQSGLERCEGEQDKRLHCY |
| | polypeptide sequence | ASWRNSSGTIELVKKGCWDDDFNCYDRQECVAT |
| | (amino acids 20-134 of | EENPQVYFCCCEGNFCNERFTHLPEAGGPEVTYEP |
| | SEQ ID NO: 28) with L79D | PPTAPTGGGTHTCPPCPAPELLGGPSVFLFPPKPKD |
| | mutation fused to an Fc | TLMISRTPEVTCVVVDVSHEDPEVKFNWYVDGVE |
| | domain and with TPA | VHNAKTKPREEQYNSTYRVVSVLTVLHQDWLNG |
| | leader sequence | KEYKCKVSNKALPAPIEKTISKAKGQPREPQVYTL |
| | | PPSREEMTKNQVSLTCLVKGFYPSDIAVEWESNG |
| | | QPENNYKTTPPVLDSDGSFFLYSKLTVDKSRWQQ |
| | | GNVFSCSVMHEALHNHYTQKSLSLSPGK* |
| 41 | human ActRIIB soluble | MDAMKRGLCCVLLLCGAVFVSPGASGRGEAETR |
| | (extracellular), processed | ECIYYNANWELERTNQSGLERCEGEQDKRLHCY |
| | polypeptide sequence | ASWANSSGTIELVKKGCWDDDFNCYDRQECVAT |
| | (amino acids 20-134 of | EENPQVYFCCCEGNFCNERFTHLPEAGGPEVTYEP |
| | SEQ ID NO: 16) with L79D | PPTAPTGGGTHTCPPCPAPELLGGPSVFLFPPKPKD |
| | mutation fused to an Fc | TLMISRTPEVTCVVVDVSHEDPEVKFNWYVDGVE |
| | domain and with TPA | VHNAKTKPREEQYNSTYRVVSVLTVLHQDWLNG |
| | leader sequence | KEYKCKVSNKALPAPIEKTISKAKGQPREPQVYTL |
| | | PPSREEMTKNQVSLTCLVKGFYPSDIAVEWESNG |
| | | QPENNYKTTPPVLDSDGSFFLYSKLTVDKSRWQQ |
| | | GNVFSCSVMHEALHNHYTQKSLSLSPGK* |
| 42 | human ActRIIB soluble | GRGEAETRECIYYNANWELERTNQSGLERCEGEQ |
| | (extracellular), processed | DKRLHCYASWRNSSGTIELVKKGCWLDDFNCYD |
| | polypeptide sequence | RQECVATEENPQVYFCCCEGNFCNERFTHLPEAG |
| | having a variant C-terminal | GPEGPWASTTIPSGGPEATAAAGDQGSGALWLCL |
| | sequence (disclosed in | EGPAHE |
| | WO2007/053775) | |
| 43 | human ActRIIB soluble | GRGEAETRECIYYNANWELERTNQSGLERCEGEQ |
| | (extracellular), processed | DKRLHCYASWRNSSGTIELVKKGCWDDDFNCYD |
| | polypeptide sequence | RQECVATEENPQVYFCCCEGNFCNERFTHLPEAG |
| | having a variant C-terminal | GPEGPWASTTIPSGGPEATAAAGDQGSGALWLCL |
| | sequence (disclosed in | EGPAHE |
| | WO2007/053775) having | |
| | an L79D mutation | |
| 44 | human ActRIIB soluble | GRGEAETRECIYYNANWELERTNQSGLERCEGEQ |
| | (extracellular), processed | DKRLHCYASWRNSSGTIELVKKGCWDDDFNCYD |
| | polypeptide sequence | RQECVATEENPQVYFCCCEGNFCNERFTHLPEAG |
| | having a variant C-terminal | GPEGPWASTTIPSGGPEATAAAGDQGSGALWLCL |
| | sequence (disclosed in | EGPAHETGGGTHTCPPCPAPELLGGPSVFLFPPKP |
| | WO2007/053775) having | KDTLMISRTPEVTCVVVDVSHEDPEVKFNWYVD |
| | an L79D mutation fused to | GVEVHNAKTKPREEQYNSTYRVVSVLTVLHQDW |
| | an Fc domain with a TGGG | LNGKEYKCKVSNKALPAPIEKTISKAKGQPREPQV |
| | linker | YTLPPSREEMTKNQVSLTCLVKGFYPSDIAVEWES |
| | | NGQPENNYKTTPPVLDSDGSFFLYSKLTVDKSRW |
| | | QQGNVFSCSVMHEALHNHYTQKSLSLSPGK* |
| 45 | Nucleic Acid Sequence | ATGGATGCAATGAAGAGAGGGCTCTGCTGTGTG |
| | Encoding SEQ ID NO: 24 | CTGCTGCTGTGTGGAGCAGTCTTCGTTTCGCCC |
| | | GGCGCCGCCGAAACCCGCGAATGTATTTATTAC |
| | | AATGCTAATTGGGAACTCGAACGGACGAACCA |
| | | ATCCGGGCTCGAACGGTGTGAGGGGGAACAGG |
| | | ATAAACGCCTCCATTGCTATGCGTCGTGGAGGA |
| | | ACTCCTCCGGGACGATTGAACTGGTCAAGAAAG |
| | | GGTGCTGGGACGACGATTTCAATTGTTATGACC |
| | | GCCAGGAATGTGTCGCGACCGAAGAGAATCCG |
| | | CAGGTCTATTTCTGTTGTTGCGAGGGGAATTTCT |
| | | GTAATGAACGGTTTACCCACCTCCCCGAAGCCG |
| | | GCGGGCCCGAGGTGACCTATGAACCCCCGCCC |
| | | ACCGGTGGTGGAACTCACACATGCCCACCGTGC |
| | | CCAGCACCTGAACTCCTGGGGGGACCGTCAGTC |
| | | TTCCTCTTCCCCCCAAAACCCAAGGACACCCTC |
| | | ATGATCTCCCGGACCCCTGAGGTCACATGCGTG |
| | | GTGGTGGACGTGAGCCACGAAGACCCTGAGGT |
| | | CAAGTTCAACTGGTACGTGGACGGCGTGGAGGT |
| | | GCATAATGCCAAGACAAAGCCGCGGGAGGAGC |
| | | AGTACAACAGCACGTACCGTGTGGTCAGCGTCC |
| | | TCACCGTCCTGCACCAGGACTGGCTGAATGGCA |
| | | AGGAGTACAAGTGCAAGGTCTCCAACAAAGCC |
| | | CTCCCAGCCCCCATCGAGAAAACCATCTCCAAA |
| | | GCCAAAGGGCAGCCCCGAGAACCACAGGTGTA |
| | | CACCCTGCCCCCATCCCGGGAGGAGATGACCA |
| | | AGAACCAGGTCAGCCTGACCTGCCTGGTCAAAG |
| | | GCTTCTATCCCAGCGACATCGCCGTGGAGTGGG |
| | | AGAGCAATGGGCAGCCGGAGAACAACTACAAG |
| | | ACCACGCCTCCCGTGCTGGACTCCGACGGCTCC |
| | | TTCTTCCTCTATAGCAAGCTCACCGTGGACAAG |
| | | AGCAGGTGGCAGCAGGGGAACGTCTTCTCATGC |
| | | TCCGTGATGCATGAGGCTCTGCACAACCACTAC |
| | | ACGCAGAAGAGCCTCTCCCTGTCCCCGGGTAAA |
| | | TGA |
| 46 | fusion protein comprising a | SGRGEAETRECIYYNANWELERTNQSGLERCEGE |
| | soluble extracellular | QDKRLHCYASWRNSSGTIELVKKGCWLDDFNCY |
| | domain of ActRIIB (R64; | DRQECVATEENPQVYFCCCEGNFCNERFTHLPEA |
| | SEQ ID NO: 29) fused to an | GGPEVTYEPPPTAPTGGGTHTCPPCPAPELLGGPS |
| | Fc domain | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPVPIEKTISKAKG |
| | | QPREPQVYTLPPSREEMTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSL |
| | | SPGK |
| 47 | fusion protein comprising a | SGRGEAETRECIYYNANWELERTNQSGLERCEGE |
| | soluble extracellular | QDKRLHCYASWRNSSGTIELVKKGCWLDDFNCY |
| | domain of ActRIIB (R64) | DRQECVATEENPQVYFCCCEGNFCNERFTHLPEA |
| | with the C-terminal 15 | GGGTHTCPPCPAPELLGGPSVFLFPPKPKDTLMIS |
| | amino acids deleted (SEQ | RTPEVTCVVVDVSHEDPEVKFNWYVDGVEVHNA |
| | ID NO: 30) fused to an Fc | KTKPREEQYNSTYRVVSVLTVLHQDWLNGKEYK |
| | domain | CKVSNKALPVPIEKTISKAKGQPREPQVYTLPPSR |
| | | EEMTKNQVSLTCLVKGFYPSDIAVEWESNGQPEN |
| | | NYKTTPPVLDSDGSFFLYSKLTVDKSRWQQGNVF |
| | | SCSVMHEALHNHYTQKSLSLSPGK |
| 48 | Exemplary human | VLSPADKTNVKAAWGKVGAHAGEYGAEALERM |
| | hemoglobin alpha subunit | FLSFPTTKTYFPHFDLSHGSAQVKGHGKKVADAL |
| | | TNAVAHVDDMPNALSALSDLHAHKLRVDPVNFK |
| | | LLSHCLLVTLAAHLPAEFTPAVHASLDKFLASVST |
| | | VLTSKYR |
| 49 | Exemplary human | GHFTEEDKATITSLWGKVNVEDAGGETLGRLLVV |
| | hemoglobin beta subunit | YPWTQRFFDSFGNLSSASAIMGNPKVKAHGKKVL |
| | | TSLGDATKHLDDLKGTFAQLSELHCDKLHVDPEN |
| | | FKLLGNVLVTVLAIHFGKEFTPEVQASWQKMVTA |
| | | VASALSSRYH |
| 50 | Exemplary human | VHLTPEEKSAVTALWGKVNVDEVGGEALGRLLV |
| | hemoglobin gamma subunit | VYPWTQRFFESFGDLSTPDAVMGNPKVKAHGKK |
| | | VLGAFSDGLAHLDNLKGTFATLSELHCDKLHVDP |
| | | ENFRLLGNVLVCVLAHHFGKEFTPPVQAAYQKV |
| | | VAGVANALAHKYH |

### 10. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed:

1. A method for treating beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject every 21 days.

2. A method for treating transfusion-dependent beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject every 21 days.

3. A method for treating non-transfusion-dependent beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject every 21 days.

4. A method for treating beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject every 21 days, wherein the genotype of the subject is selected from the group consisting of β⁰/β⁰, β⁺/β⁺, β⁺/HbE, and β⁺/HbE.

5. A method for treating beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject every 21 days, wherein the genotype of the subject comprises coinheritance of two severe hemoglobin beta chain mutations, and wherein the subject has alpha-thalassemia.

6. A method for treating beta-thalassemia in a subject in need thereof, comprising administering to the subject an initial dose of about 0.8 mg/kg or about 1.0 mg/kg of an activin receptor type II (ActRII) signaling inhibitor, wherein the activin receptor type II (ActRII) signaling inhibitor is administered subcutaneously in the upper arm, abdomen, or thigh of the subject, wherein the genotype of the subject comprises coinheritance of two severe hemoglobin beta chain mutations, and wherein the subject has hereditary persistence of fetal hemoglobin.

7. A method for treating beta-thalassemia in a subject in need thereof, comprising administering to the subject an initial dose of about 0.8 mg/kg or about 1.0 mg/kg of an activin receptor type II (ActRII) signaling inhibitor, and subsequently administering the ActRII signaling inhibitor to the subject one or more times at 21 day intervals, such that the beta-thalassemia is treated, wherein said administering comprises administering subcutaneously in the upper arm, abdomen, or thigh of the subject.

8. A method for treating beta-thalassemia in a subject in need thereof, comprising administering to the subject an initial dose of about 0.8 mg/kg or about 1.0 mg/kg of an activin receptor type II (ActRII) signaling inhibitor, and subsequently administering the ActRII signaling inhibitor to the subject one or more times at 21 day intervals, such that the beta-thalassemia is treated, wherein said administering comprises administering subcutaneously in the upper arm, abdomen, or thigh of the subject, and wherein the genotype of the subject is selected from the group consisting of β⁰/β⁰, β⁺/β⁺, β⁰/β⁺, β⁰/HbE, and β⁺/HbE.

9. A method for treating beta-thalassemia in a subject in need thereof, comprising administering to the subject an initial dose of 0.8 mg/kg or about 1.0 mg/kg of an activin receptor type II (ActRII) signaling inhibitor, and subsequently administering the ActRII signaling inhibitor one or more times at 21 day intervals, such that the beta-thalassemia is treated, wherein said administering comprises administering subcutaneously in the upper arm, abdomen, or thigh of the subject, and wherein the subject has hereditary persistence of fetal hemoglobin.

10. A method for treating beta-thalassemia in a subject in need thereof, comprising administering to the subject an initial dose of about 0.8 mg/kg or about 1.0 mg/kg of an activin receptor type II (ActRII) signaling inhibitor, and subsequently administering the ActRII signaling inhibitor to the subject one or more times at 21 day intervals, such that the beta-thalassemia is treated, wherein said administering comprises administering subcutaneously in the upper arm, abdomen, or thigh of the subject, and wherein said administering is sufficient to detectably reduce GDF-11 levels in serum from said subject between administrations.

11. The method of any of claims 6-10, wherein the beta-thalassemia is transfusion-dependent beta-thalassemia.

12. The method of any of claims 6-10, wherein the beta-thalassemia is non-transfusion-dependent beta-thalassemia.

13. The method of any of claims 1-12, further comprising taking a first measurement of hemoglobin concentration in the subject; after a first period of time taking a second measurement of hemoglobin concentration in the subject; and administering a subsequent dose of the ActRII signaling inhibitor based on the difference between the second measurement of hemoglobin concentration and the first measurement of hemoglobin concentration, wherein said administering comprises administering subcutaneously in the upper arm, abdomen, or thigh or the subject.

14. The method of any of claims 1-12, further comprising taking a first measurement of hematocrit in the subject; after a first period of time taking a second measurement of hematocrit in the subject; and administering a subsequent dose of the ActRII signaling inhibitor based on the difference between the second measurement of hematocrit and the first measurement of hematocrit, wherein said administering comprises administering subcutaneously in the upper arm, abdomen, or thigh or the subject.

15. The method of any of claims 1-12, further comprising taking a first measurement of fetal hemoglobin in the subject; after a first period of time taking a second measurement of fetal hemoglobin concentration in the subject; and administering a subsequent dose of the ActRII signaling inhibitor based on the difference between the second measurement of fetal hemoglobin concentration and the first measurement of fetal hemoglobin concentration, wherein said administering comprises administering subcutaneously in the upper arm, abdomen, or thigh or the subject.

16. The method of any of claims 1-12, further comprising
(a) taking a first measurement of hemoglobin concentration, hematocrit, or fetal hemoglobin concentration in the subject
(b) after a first period of time taking a second measurement of hemoglobin concentration, hematocrit, or fetal hemoglobin concentration in the subject;
(c) after a second period of time, discontinuing administration of the initial dose and administering to the subject a subsequent dose of the ActRII signaling inhibitor, wherein the subsequent dose is administered via subcutaneous injection in the upper arm, abdomen or thigh of the subject.

17. The method of any of claims 11-16, wherein the first measurement of hemoglobin concentration, hematocrit, or fetal hemoglobin concentration is taken prior to administering to the subject the initial dose the ActRII signaling inhibitor.

18. The method of any of claims 11-17, wherein the first measurement of hemoglobin concentration, hematocrit, or fetal hemoglobin concentration is immediately after the initial dose the ActRII signaling inhibitor is administered to the subject or within at most 1 day, 2 days, 3 days, 4 days, 5 days, 6 days, or 1 week thereof.

19. The method of any one of claims 11-18, wherein the second measurement of hemoglobin, hematocrit, or fetal hemoglobin concentration is taken approximately 3 weeks, 1 month, 2 months, 3 months, 4 months, 5 months, 6 months, 7 months, 8 months, 9 months, 10 months, 11 months, or 12 months after the initial dose the ActRII signaling inhibitor is administered to the subject.

20. The method of any one of claims 11-19, wherein the second period of time is within 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, or 12 weeks of the when the second measurement is taken.

21. The method of any one of claims 11-20, wherein the subsequent dose of the ActRII signaling inhibitor is about 0.3 mg/kg, about 0.45 mg/kg, about 0.6 mg/kg, about 1.0 mg/kg, or about 1.25 mg/kg.

22. The method of any one of claims 11-21, wherein the method further comprises taking a third measurement of hemoglobin concentration, hematocrit, or fetal hemoglobin concentration in the subject.

23. The method of any one of claims 16-20, wherein
(a) the second measurement of hemoglobin concentration is less than or equal to 12.5 g/dL;
(b) the second measurement of hemoglobin concentration is less than or equal to 1.5 g/dL greater than the first measurement of hemoglobin concentration; and
(c) the subsequent dose is equal to the initial dose.

24. The method of any one of claims 16-20, wherein
(a) the second measurement of hemoglobin concentration is less than or equal to 12.5 g/dL;
(b) the second measurement of hemoglobin concentration is greater than 1.5 g/dL greater than the first measurement of hemoglobin concentration; and
(c) the subsequent dose is approximately 25% less than the initial dose.

25. The method of any one of claims 16-20, wherein
(a) the second measurement of hemoglobin concentration is (i) greater than 12.5 g/dL and less than or equal to 14 g/dL; and (ii) less than or equal to 1.5 g/dL greater than the first measurement of hemoglobin concentration;
(b) the subsequent dose is equal to the initial dose; and
(c) the second period of time consists of a dose delay of up to twelve weeks until a third measurement of hemoglobin concentration is less than or equal to 12.5 g/dL.

26. The method of any one of claims 16-20, wherein
(a) the second measurement of hemoglobin concentration is (i) greater than 12.5 g/dL and less than or equal to 14 g/dL, and (ii) greater than 1.5 g/dL greater than the first measurement of hemoglobin concentration;
(b) the subsequent dose is approximately 25% less than the initial dose; and
(c) the second period of time consists of a dose delay of up to twelve weeks until a third measurement of hemoglobin concentration is determined to be (i) less than or equal to 12.5 g/dL, and (ii) the change between the first measurement of hemoglobin concentration and the third measurement of hemoglobin concentration is less than or equal to 1.5 g/dL.

27. The method of any one of claims 16-20, wherein
(a) the second measurement of hemoglobin concentration is greater than 14 g/dL;
(b) the subsequent dose is approximately 25% less than the initial dose; and
(c) the second period of time consists of a dose delay of up to twelve weeks until a third measurement of hemoglobin concentration is less than 12.5 g/dL.

28. The method of any of claims 1-27, wherein the initial dose is administered once every 21 days.

29. The method of any of claims 11-28, wherein the subsequent dose is administered once every 21 days.

30. The method of any one of the preceding claims, wherein the method further comprises decreasing GDF11 levels in the subject.

31. The method of any of the preceding claims, wherein the method further comprises increasing fetal hemoglobin levels in the subject.

32. The method of any of claims 1-31, wherein the ActRII signaling inhibitor is an inhibitor of ActRIIA signaling.

33. The method of any of claims 1-31, wherein the ActRII signaling inhibitor is a humanized fusion-protein consisting of the extracellular domain of ActRIIA and the human IgG1 Fc domain.

34. The method of claim 32, wherein the ActRIIA signaling inhibitor is a polypeptide comprising an amino acid sequence selected from the group consisting of:
(a) 90% identical to SEQ ID NO:2;
(b) 95% identical to SEQ ID NO:2;
(c) 98% identical to SEQ ID NO:2;
(d) SEQ ID NO:2;
(e) 90% identical to SEQ ID NO:3;
(f) 95% identical to SEQ ID NO:3;
(g) 98% identical to SEQ ID NO:3;
(h) SEQ ID NO:3;
(i) 90% identical to SEQ ID NO:6;
(j) 95% identical to SEQ ID NO:6;
(k) 98% identical to SEQ ID NO:6;
(l) SEQ ID NO:6;
(m) 90% identical to SEQ ID NO:7;
(n) 95% identical to SEQ ID NO:7;
(o) 98% identical to SEQ ID NO:7; and
(p) SEQ ID NO:7.

35. The method of claim 32, wherein the ActRII signaling inhibitor is a polypeptide comprising the amino acid sequence of SEQ ID NO:7.

36. The method of any of claims 1-31, wherein the ActRII signaling inhibitor is an inhibitor of ActRIIB signaling.

37. The method of any of claims 1-31, wherein the ActRII signaling inhibitor is a humanized fusion-protein consisting of the extracellular domain of ActRIIB and the human IgG1 Fc domain.

38. The method of claim 36, wherein the ActRIIB inhibitor is a polypeptide comprising an amino acid sequence selected from the group consisting of:
(a) 90% identical to SEQ ID NO:17;
(b) 95% identical to SEQ ID NO:17;
(c) 98% identical to SEQ ID NO:17;
(d) SEQ ID NO:17;
(e) 90% identical to SEQ ID NO:20;
(f) 95% identical to SEQ ID NO:20;
(g) 98% identical to SEQ ID NO:20;
(h) SEQ ID NO:20;
(i) 90% identical to SEQ ID NO:21;
(j) 95% identical to SEQ ID NO:21;
(k) 98% identical to SEQ ID NO:21;
(l) SEQ ID NO:21;
(m) 90% identical to SEQ ID NO:25;
(n) 95% identical to SEQ ID NO:25;
(o) 98% identical to SEQ ID NO:25; and
(p) SEQ ID NO:25.

39. The method of claim 36, wherein the ActRIIB signaling inhibitor is a polypeptide comprising the amino acid sequence of SEQ ID NO:25.

40. A method for treating beta-thalassemia in a subject in need thereof, comprising administering to the subject an initial dose of about 0.8 mg/kg or about 1.0 mg/kg of an activin receptor type IIB (ActRIIB) signaling inhibitor, wherein the activin receptor type IIB (ActRIIB) signaling inhibitor is administered subcutaneously in the upper arm, abdomen, or thigh of the subject every 21 days, and wherein the ActRIIB signaling inhibitor comprises the amino acid sequence of SEQ ID NO:25.

41. A method for treating transfusion-dependent beta-thalassemia in a subject in need thereof, comprising administering to the subject an initial dose of about 0.8 mg/kg or about 1.0 mg/kg of an activin receptor type IIB (ActRIIB) signaling inhibitor, wherein the activin receptor type II (ActRIIB) signaling inhibitor is administered subcutaneously in the upper arm, abdomen, or thigh of the subject every 21 days, and wherein the ActRIIB signaling inhibitor comprises the amino acid sequence of SEQ ID NO:25.

42. A method for treating non-transfusion-dependent beta-thalassemia in a subject in need thereof, comprising administering to the subject an initial dose of about 0.8 mg/kg or about 1.0 mg/kg of an activin receptor type IIB (ActRIIB) signaling inhibitor, wherein the activin receptor type IIB (ActRIIB) signaling inhibitor is administered subcutaneously in the upper arm, abdomen, or thigh of the subject every 21 days, and wherein the ActRIIB signaling inhibitor comprises the amino acid sequence of SEQ ID NO:25.

43. A method for treating beta-thalassemia in a subject in need thereof, comprising administering to the subject an initial dose of about 0.8 mg/kg or about 1.0 mg/kg of an activin receptor type IIB (ActRIIB) signaling inhibitor, wherein the activin receptor type IIB (ActRIIB) signaling inhibitor is administered subcutaneously in the upper arm, abdomen, or thigh of the subject every 21 days, wherein the genotype of the subject is selected from the group consisting of β⁰/β⁰, β⁺/β⁺, β⁰/β⁺, β⁰/HbE, and β⁺/HbE, and wherein the ActRIIB signaling inhibitor comprises the amino acid sequence of SEQ ID NO:25.

44. A method for treating beta-thalassemia in a subject in need thereof, comprising administering to the subject an initial dose of about 0.8 mg/kg or about 1.0 mg/kg of an activin receptor type IIB (ActRIIB) signaling inhibitor, wherein the activin receptor type IIB (ActRIIB) signaling inhibitor is administered subcutaneously in the upper arm, abdomen, or thigh of the subject every 21 days, wherein the genotype of the subject comprises coinheritance of two severe hemoglobin beta chain mutations, wherein the subject has alpha-thalassemia, and wherein the ActRIIB signaling inhibitor comprises the amino acid sequence of SEQ ID NO:25.]

45. A method for treating beta-thalassemia in a subject in need thereof, comprising administering to the subject an initial dose of about 0.8 mg/kg or about 1.0 mg/kg of an activin receptor type IIB (ActRIIB) signaling inhibitor, wherein the activin receptor type IIB (ActRIIB) signaling inhibitor is administered subcutaneously in the upper arm, abdomen, or thigh of the subject every 21 days, wherein the genotype of the subject comprises coinheritance of two severe hemoglobin beta chain mutations, wherein the subject has hereditary persistence of fetal hemoglobin, and wherein the ActRIIB signaling inhibitor comprises the amino acid sequence of SEQ ID NO:25.

46. A method for treating beta-thalassemia in a subject in need thereof, comprising administering to the subject an initial dose of about 0.8 mg/kg or about 1.0 mg/kg of an activin receptor type IIB (ActRIIB) signaling inhibitor, and subsequently administering the ActRIIB signaling inhibitor to the subject one or more times at 21 day intervals, such that the beta-thalassemia is treated, wherein said administering comprises administering subcutaneously in the upper arm, abdomen, or thigh of the subject.

47. A method for treating beta-thalassemia in a subject in need thereof, comprising administering to the subject an initial dose of about 0.8 mg/kg or about 1.0 mg/kg of an activin receptor type IIB (ActRIIB) signaling inhibitor, and subsequently administering the ActRIIB signaling inhibitor to the subject one or more times at 21 day intervals, such that the beta-thalassemia is treated, wherein said administering comprises administering subcutaneously in the upper arm, abdomen, or thigh of the subject, and wherein the genotype of the subject is selected from the group consisting of β⁰/β⁰, β⁺/β⁺, β⁰/β⁺, β⁰/HbE, and β⁺/HbE.

48. A method for treating beta-thalassemia in a subject in need thereof, comprising administering to the subject an initial dose of about 0.8 mg/kg or about 1.0 mg/kg of an activin receptor type IIB (ActRIIB) signaling inhibitor, and subsequently administering the ActRIIB signaling inhibitor one or more times at 21 day intervals, such that the beta-thalassemia is treated, wherein said administering comprises administering subcutaneously in the upper arm, abdomen, or thigh of the subject, and wherein the subject has hereditary persistence of fetal hemoglobin.

49. A method for treating beta-thalassemia in a subject in need thereof, comprising administering to the subject an initial dose of about 0.8 mg/kg or about 1.0 mg/kg of an activin receptor type IIB (ActRIIB) signaling inhibitor, and subsequently administering the ActRIIB signaling inhibitor to the subject one or more times at 21 day intervals, such that the beta-thalassemia is treated, wherein said administering comprises administering subcutaneously in the upper arm, abdomen, or thigh of the subject, and wherein said administering is sufficient to detectably reduce GDF-11 levels in serum from said subject between administrations.

50. The method of any of claims 46-49, wherein the beta-thalassemia is transfusion-dependent beta-thalassemia.

51. The method of any of claims 46-49, wherein the beta-thalassemia is non-transfusion-dependent beta-thalassemia.

52. The method of any of claims 40-51, further comprising taking a first measurement of hemoglobin concentration in the subject; after a first period of time taking a second measurement of hemoglobin concentration in the subject; and administering a subsequent dose of the ActRIIB signaling inhibitor based on the difference between the second measurement of hemoglobin concentration and the first measurement of hemoglobin concentration, wherein said administering comprises administering subcutaneously in the upper arm, abdomen, or thigh or the subject.

53. The method of any of claims 40-51, further comprising taking a first measurement of hematocrit in the subject; after a first period of time taking a second measurement of hematocrit in the subject; and administering a subsequent dose of the ActRIIB signaling inhibitor based on the difference between the second measurement of hematocrit and the first measurement of hematocrit, wherein said administering comprises administering subcutaneously in the upper arm, abdomen, or thigh or the subject.

54. The method of any of claims 40-51, further comprising taking a first measurement of fetal hemoglobin concentration in the subject; after a first period of time taking a second measurement of fetal hemoglobin concentration in the subject; and administering a subsequent dose of the ActRIIB signaling inhibitor based on the difference between the second measurement of fetal hemoglobin concentration and the first measurement of fetal hemoglobin concentration, wherein said administering comprises administering subcutaneously in the upper arm, abdomen, or thigh or the subject.

55. The method of any of claims 40-51, further comprising
(a) taking a first measurement of hemoglobin concentration in the subject
(b) after a first period of time taking a second measurement of hemoglobin concentration in the subject;
(c) after a second period of time, discontinuing administration of the initial dose and administering to the subject a subsequent dose of the ActRIIB signaling inhibitor, wherein the subsequent dose is administered via subcutaneous injection in the upper arm, abdomen or thigh of the subject.

56. The method of any of claims 52-55, wherein the first measurement of hemoglobin concentration, hematocrit, or fetal hemoglobin concentration is taken prior to administering to the subject the initial dose the ActRIIB signaling inhibitor.

57. The method of any of claims 52-56, wherein the first measurement of hemoglobin concentration, hematocrit, or fetal hemoglobin concentration is immediately after the initial dose the ActRIIB signaling inhibitor is administered to the subject or within at most 1 day, 2 days, 3 days, 4 days, 5 days, 6 days, or 1 week thereof.

58. The method of any one of claims 52-57, wherein the second measurement of hemoglobin concentration, hematocrit, or fetal hemoglobin concentration is taken approximately 3 weeks, 1 month, 2 months, 3 months, 4 months, 5 months, 6 months, 7 months, 8 months, 9 months, 10 months, 11 months, or 12 months after the initial dose the ActRIIB signaling inhibitor is administered to the subject.

59. The method of any one of claims 52-58, wherein the second period of time is within 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, or 12 weeks of the when the second measurement is taken.

60. The method of any one of claims 52-59, wherein the subsequent dose of the ActRIIB signaling inhibitor is about 0.3 mg/kg, about 0.45 mg/kg, about 0.6 mg/kg, about 1.0 mg/kg, or about 1.25 mg/kg.

61. The method of any one of claims 52-60, wherein the method further comprises taking a third measurement of hemoglobin concentration, hematocrit, or fetal hemoglobin concentration in the subject.

62. The method of any one of claims 52-59, wherein
(a) the second measurement of hemoglobin concentration is less than or equal to 12.5 g/dL;
(b) the second measurement of hemoglobin concentration is less than or equal to 1.5 g/dL greater than the first measurement of hemoglobin concentration; and
(c) the subsequent dose is equal to the initial dose.

63. The method of any one of claims 52-59, wherein
(a) the second measurement of hemoglobin concentration is less than or equal to 12.5 g/dL;
(b) the second measurement of hemoglobin concentration is greater than 1.5 g/dL greater than the first measurement of hemoglobin concentration; and
(c) the subsequent dose is approximately 25% less than the initial dose.

64. The method of any one of claims 52-59, wherein
(a) the second measurement of hemoglobin concentration is (i) greater than 12.5 g/dL and less than or equal to 14 g/dL; and (ii) less than or equal to 1.5 g/dL greater than the first measurement of hemoglobin concentration;
(b) the subsequent dose is equal to the initial dose; and
(c) the second period of time consists of a dose delay of up to twelve weeks until a third measurement of hemoglobin concentration is less than or equal to 12.5 g/dL.

65. The method of any one of claims 52-59, wherein
(a) the second measurement of hemoglobin concentration is (i) greater than 12.5 g/dL and less than or equal to 14 g/dL, and (ii) greater than 1.5 g/dL greater than the first measurement of hemoglobin concentration;
(b) the subsequent dose is approximately 25% less than the initial dose; and
(c) the second period of time consists of a dose delay of up to twelve weeks until a third measurement of hemoglobin concentration is determined to be (i) less than or equal to 12.5 g/dL, and (ii) the change between the first measurement of hemoglobin concentration and the third measurement of hemoglobin concentration is less than or equal to 1.5 g/dL.

66. The method of any one of claims 52-59, wherein
(a) the second measurement of hemoglobin concentration is greater than 14 g/dL;
(b) the subsequent dose is approximately 25% less than the initial dose; and
(c) the second period of time consists of a dose delay of up to twelve weeks until a third measurement of hemoglobin concentration is less than 12.5 g/dL.

67. The method of any of claims 40-66, wherein the initial dose is administered once every 21 days.

68. The method of any of claims 52-67, wherein the subsequent dose is administered once every 21 days.

69. The method of any one of claims 40-68, wherein the method further comprises decreasing GDF11 levels in the subject.

70. The method of any one of claims 40-69, wherein the method further comprises increasing fetal hemoglobin levels in the subject.

71. A method of increasing fetal hemoglobin levels in a subject comprising administering an ActRIIB signaling inhibitor to the subject.

72. The method of any of the preceding claims, wherein the subject expresses hemoglobin E.

73. The method of any of the preceding claims, wherein the subject does not express hemoglobin S.

74. The method of any of the preceding claims, wherein the erythroid response consists of (i) a greater than or equal to 33% reduction in transfusion burden for 12 weeks, and (ii) a reduction of at least 2 units of red blood cells over a 12 week period.

75. The method of any of claims 1-73, wherein the erythroid response consists of a greater than 1 g/dL increase in hemoglobin concentration as compared to a baseline hemoglobin concentration, wherein the increase in hemoglobin concentration is measured by the mean of hemoglobin concentration values over a contiguous 12-week period in the absence of transfusion.

76. The method of any one of the preceding claims, wherein the subject is a human

77. The method of any one of the preceding claims, wherein the ActRII signaling inhibitor is packaged in a container as a sterile, preservative-free lyophilized cake, stored between 2° C. and 8° C. prior to administration to the subject.

78. The method of claim 77, wherein the container contains 37.5 mg of the ActRII signaling inhibitor.

79. The method of claim 77, wherein the container contains 75 mg of the ActRII signaling inhibitor.
